(19) United States
(12) Patent Application Publication
(10) Pub. No.: US 2021/0386791 A1
(43) Pub. Date: Dec. 16, 2021

(54) METHODS OF CELLULAR REPROGRAMMING

(71) Applicant: TissueTech, Inc., Miami, FL (US)

(72) Inventors: Scheffer TSENG, Pinecrest, FL (US); Frank E. YOUNG, Miami, FL (US); Ying-Tieng ZHU, Miami, FL (US); Szu Yu CHEN, Miami, FL (US)

(21) Appl. No.: 17/291,070

(22) PCT Filed: Nov. 6, 2019
(86) PCT No.: PCT/US2019/060140

§ 371 Date: May 4, 2021

Related U.S. Application Data
(60) Provisional application No. 62/757,082, filed on Nov. 7, 2018.

(51) Int. Cl. A61K 35/51; A61K 35/30; A61K 35/33; A61K 35/34; A61K 35/50; A61K 35/545; A61K 47/10; A61K 9/19

(52) U.S. Cl. CPC A61K 35/51; A61K 35/30; A61K 35/33; A61K 9/19; A61K 35/50; A61K 35/545; A61K 47/10; A61K 35/34

(57) ABSTRACT

Disclosed herein are methods of cellular reprogramming, comprising contacting a cell with HC-HA/PTX3 for a time sufficient for cellular reprogramming of the phenotype of the cell to a different phenotype.

### CROSS-REFERENCE

This application claims the benefit of U.S. Provisional Application No. 62/757,082, filed Nov. 7, 2018, which application is incorporated herein by reference in its entirety.

### STATEMENT AS TO FEDERALLY SPONSORED RESEARCH

This invention was made with the support of the United States government under Contract number RO1EY06819 awarded by National Eye Institute of the National Institutes of Health. The government has certain rights in the invention.

### SUMMARY OF THE DISCLOSURE

Provided herein in some aspects are methods of reprogramming a cell having a first phenotype, comprising: contacting the cell with HC-HA/PTX3 for a time sufficient to reprogram the first phenotype of the cell to second phenotype. In some embodiments, the second phenotype corresponds to a phenotype of an earlier cell in a cellular differentiation pathway. In some embodiments, the cell is reprogrammed into an earlier cell in a cellular differentiation pathway. In some embodiments, the cell is a cell differentiated from a progenitor cell. In some embodiments, the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell. In some embodiments, the progenitor cell is a neural crest progenitor. In some embodiments, the cell differentiated from the progenitor cell is a mesenchymal cell. In some embodiments, the cell differentiated from the progenitor cell is a fibroblast, myofibroblast, keratocyte, epithelial cell, or limbal niche cell. In some embodiments, the fibroblast is a myofibroblast, a dermal fibroblast, a corneal fibroblast, or a cardiac fibroblast. In some embodiments, the earlier cell is the progenitor cell. In some embodiments, the cell is present in a tissue following damage or degeneration of the tissue. In some embodiments, the tissue is ocular, cardiac, skin, joint, spine, soft tissue, cartilage, bone, tendon, ligament, nerve, intervertebral disc, spinal cord, brain, or muscle tissue. In some embodiments, the tissue is cardiac tissue. In some embodiments, the tissue is ocular tissue. In some embodiments, the damage is the result of a burn, a laceration, ischemic tissue, a wound, an injury, an ulcer, radiation, chemotherapy, or a surgical incision. In some embodiments, the injury is a myocardial infarction. In some embodiments, the HC-HA/PTX3 is comprised in a preparation of a fetal support tissue. In some embodiments, the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof. In some embodiments, the fetal support tissue is selected from placenta, placental amniotic membrane, umbilical cord, umbilical cord amniotic membrane, chorion, amnion-chorion, amniotic stroma, amniotic jelly, or a combination thereof. In some embodiments, the fetal support tissue is frozen or previously frozen. In some embodiments, the fetal support tissue is substantially free of red blood cells. In some embodiments, the fetal support tissue comprises umbilical cord substantially free of a vein or artery. In some embodiments, the fetal support tissue comprises cells, substantially all of which are dead. In some embodiments, the fetal support tissue comprises umbilical cord amniotic membrane and at least a portion of Wharton's Jelly. In some embodiments, the fetal support tissue is cryopreserved, lyophilized, sterilized, or a combination thereof. In some embodiments, the composition is a gel, a solution, or a suspension. In some embodiments, the HC-HA/PTX3 is native HC-HA/PTX3, reconstituted HC-HA/PTX3, or a combination thereof. In some embodiments, the method further comprises contacting the fibroblastic cell with TGFβ1.

Provided herein in some aspects are methods of treating a condition characterized by unwanted fibroblastic cell differentiation in a subject in need thereof comprising, contacting a fibroblastic cell within a tissue affected by the condition in the subject with HC-HA/PTX3 for a period of time sufficient to reprogram a phenotype of the fibroblastic cell to a different phenotype, thereby treating the condition. In some embodiments, the different phenotype corresponds to a phenotype of an earlier cell in a cellular differentiation pathway. In some embodiments, the fibroblastic cell is reprogrammed into an earlier cell in a cellular differentiation pathway. In some embodiments, the fibroblastic cell is a cell differentiated from a progenitor cell. In some embodiments, the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell. In some embodiments, the progenitor cell is a neural crest progenitor. In some embodiments, the cell differentiated from the progenitor cell is a mesenchymal cell. In some embodiments, the cell differentiated from the progenitor cell is a fibroblast, myofibroblast, keratocyte, epithelial cell, or limbal niche cell. In some embodiments, the fibroblast is a myofibroblast, a dermal fibroblast, a corneal fibroblast, or a cardiac fibroblast. In some embodiments, the earlier cell is the progenitor cell. In some embodiments, the tissue is ocular, cardiac, skin, joint, spine, soft tissue, cartilage, bone, tendon, ligament, nerve, intervertebral disc, spinal cord, brain, or muscle tissue. In some embodiments, the tissue is ocular tissue. In some embodiments, the tissue is cardiac tissue. In some embodiments, the condition is myocardial infarction. In some embodiments, the contacting occurs during a stent placement surgical procedure. In some embodiments, the condition occurs as the result of a burn, a laceration, ischemic tissue, a wound, an injury, an ulcer, radiation, chemotherapy, or a surgical incision. In some embodiments, HC-HA/PTX3 is comprised in a preparation of fetal support tissue. In some embodiments, the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof. In some embodiments, the fetal support tissue is selected from placenta, placental amniotic membrane, umbilical cord, umbilical cord amniotic membrane, chorion, amnion-chorion, amniotic stroma, amniotic jelly, or a combination thereof. In some embodiments, the fetal support tissue is frozen or previously frozen. In some embodiments, the fetal support tissue is substantially free of red blood cells. In some embodiments, the fetal support tissue comprises umbilical cord substantially free of a vein or artery. In some embodiments, the fetal support tissue comprises cells, substantially all of which are dead. In some embodiments, the fetal support tissue comprises umbilical cord amniotic membrane and at least a portion of Wharton's Jelly. In some embodiments, the fetal support tissue is cryopreserved, lyophilized, sterilized, or a combination thereof. In some embodiments, the composition is a gel, a solution, or a suspension. In some embodiments, the HC-HA/PTX3 is native HC-HA/PTX3, reconstituted HC-HA/PTX3, or a combination thereof. In some embodiments, the method further comprises contacting the fibroblastic cell with TGFβ1.

Provided herein, in some aspects, are methods of reversing a disease state in a tissue comprising, contacting the tissue with HC-HA/PTX3 for a time sufficient to reprogram diseased or unwanted cells in the tissue a cell having a different phenotype, thereby reversing the disease state of the tissue. In some embodiments, the different phenotype corresponds to a phenotype of an earlier cell in a cellular differentiation pathway. In some embodiments, the different phenotype corresponds to a phenotype of a progenitor cell. In some embodiments, the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell. In some embodiments, the unwanted cell is a fibroblast, myofibroblast, keratocyte, epithelial cell, or limbal niche cell. In some embodiments, the fibroblast is a myofibroblast, a dermal fibroblast, a corneal fibroblast, or a cardiac fibroblast. In some embodiments, the disease or unwanted cell is present in a tissue following scarring, damage, or degeneration of the tissue. In some embodiments, the tissue is ocular, cardiac, skin, joint, spine, soft tissue, cartilage, bone, tendon, ligament, nerve, intervertebral disc, spinal cord, brain, or muscle tissue. In some embodiments, the tissue is cardiac tissue. In some embodiments, the tissue is ocular tissue. In some embodiments, the HC-HA/PTX3 is comprised in a preparation of a fetal support tissue. In some embodiments, the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof. In some embodiments, the fetal support tissue is selected from placenta, placental amniotic membrane, umbilical cord, umbilical cord amniotic membrane, chorion, amnion-chorion, amniotic stroma, amniotic jelly, or a combination thereof. In some embodiments, the fetal support tissue comprises cells, substantially all of which are dead. In some embodiments, the fetal support tissue comprises umbilical cord amniotic membrane and at least a portion of Wharton's Jelly. In some embodiments, the fetal support tissue is cryopreserved, lyophilized, sterilized, or a combination thereof. In some embodiments, the HC-HA/PTX3 is native HC-HA/PTX3, reconstituted HC-HA/PTX3, or a combination thereof.

Provided herein, in some aspects, are methods of producing a progenitor cell from a differentiated cell comprising, contacting the differentiated cell with HC-HA/PTX3 for a time sufficient to reprogram the differentiated cell to a progenitor cell phenotype. In some embodiments, the progenitor cell phenotype corresponds to a phenotype of an earlier cell in a cellular differentiation pathway. In some embodiments, the progenitor cell phenotype corresponds the that of a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell. In some embodiments, the differentiated cell is a fibroblast, myofibroblast, keratocyte, epithelial cell, or limbal niche cell. In some embodiments, the fibroblast is a myofibroblast, a dermal fibroblast, a corneal fibroblast, or a cardiac fibroblast. In some embodiments, the differentiated cell is present in a tissue following scarring, damage, or degeneration of the tissue. In some embodiments, the tissue is ocular, cardiac, skin, joint, spine, soft tissue, cartilage, bone, tendon, ligament, nerve, intervertebral disc, spinal cord, brain, or muscle tissue. In some embodiments, the tissue is cardiac tissue. In some embodiments, the tissue is ocular tissue. In some embodiments, the HC-HA/PTX3 is comprised in a preparation of a fetal support tissue. In some embodiments, the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof. In some embodiments, the fetal support tissue is selected from placenta, placental amniotic membrane, umbilical cord, umbilical cord amniotic membrane, chorion, amnion-chorion, amniotic stroma, amniotic jelly, or a combination thereof. In some embodiments, the fetal support tissue comprises cells, substantially all of which are dead. In some embodiments, the fetal support tissue comprises umbilical cord amniotic membrane and at least a portion of Wharton's Jelly. In some embodiments, the fetal support tissue is cryopreserved, lyophilized, sterilized, or a combination thereof. In some embodiments, the HC-HA/PTX3 is native HC-HA/PTX3, reconstituted HC-HA/PTX3, or a combination thereof.

Provided herein in some aspects are methods of regenerating a tissue comprising, reprogramming a first differentiated phenotype of a cell within a tissue to a progenitor phenotype, and differentiating the progenitor phenotype into a second differentiated phenotype, thereby regenerating the tissue. In some embodiments, the method is performed in vitro. In some embodiments, the method is performed in vivo. In some embodiments, the method is performed ex vivo. In some embodiments, the progenitor cell phenotype corresponds to a phenotype of an earlier cell in a cellular differentiation pathway. In some embodiments, the progenitor cell phenotype corresponds the that of a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell. In some embodiments, the first differentiated cell is a fibroblast, myofibroblast, keratocyte, epithelial cell, or limbal niche cell. In some embodiments, the fibroblast is a myofibroblast, a dermal fibroblast, a corneal fibroblast, or a cardiac fibroblast. In some embodiments, the first differentiated cell is present in the tissue following scarring, damage, or degeneration of the tissue. In some embodiments, the tissue is ocular, cardiac, skin, joint, spine, soft tissue, cartilage, bone, tendon, ligament, nerve, intervertebral disc, spinal cord, brain, or muscle tissue. In some embodiments, the tissue is cardiac tissue. In some embodiments, the tissue is ocular tissue. In some embodiments, the HC-HA/PTX3 is comprised in a preparation of a fetal support tissue. In some embodiments, the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof. In some embodiments, the fetal support tissue is selected from placenta, placental amniotic membrane, umbilical cord, umbilical cord amniotic membrane, chorion, amnion-chorion, amniotic stroma, amniotic jelly, or a combination thereof. In some embodiments, the fetal support tissue comprises cells, substantially all of which are dead. In some embodiments, the fetal support tissue comprises umbilical cord amniotic membrane and at least a portion of Wharton's Jelly. In some embodiments, the fetal support tissue is cryopreserved, lyophilized, sterilized, or a combination thereof. In some embodiments, the HC-HA/PTX3 is native HC-HA/PTX3, reconstituted HC-HA/PTX3, or a combination thereof.

Provided herein in some aspects are compositions comprising a) HC-HA/PTX3 and b) a therapeutic cell. In some embodiments, the HC-HA/PTX3 is in an amount sufficient to maintain the therapeutic cell in a pluripotent state. In some embodiments, the therapeutic cell is a progenitor cell, a stem cell, or an induced pluripotent stem cell. In some embodiments, the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell. In some embodiments, HC-HA/PTX3 is comprised in a preparation of fetal support tissue. In some embodiments, the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof. In some embodiments, the fetal support tissue is selected from placenta, placental amniotic membrane, umbilical cord, umbilical cord amniotic membrane, chorion, amnion-chorion, amniotic stroma, amniotic jelly, or a combination thereof. In some embodiments, the HC-HA/PTX3 is native HC-HA/PTX3, reconstituted HC-HA/PTX3, or a combination thereof.

Disclosed herein, in some embodiments, are methods of regenerating a tissue having unwanted changes comprising: contacting a fibroblastic cell within a tissue comprising mesenchymal cells characteristic of the tissue and abnormal fibroblastic cells with HC-HA/PTX3 for a time sufficient to reprogram the fibroblastic cell to a progenitor cell or a normal mesenchymal cell characteristic of the tissue. In some instances, the tissue is not scar tissue. In some embodiments, the HC-HA/PTX3 is comprised in a composition comprising: (a) a preparation comprising HC-HA/PTX3; and (b) a pharmaceutically acceptable diluent, excipient, vehicle, or carrier. In some instances, the tissue is scar tissue. In some instances, the abnormal fibroblastic cells are generated by degenerative disease, aging, scarring, wound, burn, radiation, chemotherapy, surgical incision, laceration, ulceration, injury, or ischemia. In some instances, the fibroblastic cell is a fibroblast, a myofibroblast, a dermal fibroblast, a corneal fibroblast, or a cardiac fibroblast. In some instances, the fibroblastic cell is not a myofibroblast differentiated from an amniotic membrane stromal cell. In some instances, the myofibroblast is abnormally differentiated. In some instances, the myofibroblast is present in the tissue following damage or degeneration of the tissue. In some instances, the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof. In some instances, the fetal support tissue is selected from placenta, placental amniotic membrane, umbilical cord, umbilical cord amniotic membrane, chorion, amnion-chorion, amniotic stroma, amniotic jelly, or a combination thereof. In some instances, the fetal support tissue is frozen or previously frozen. In some instances, the fetal support tissue is substantially free of red blood cells. In some instances, the fetal support tissue comprises umbilical cord substantially free of a vein or artery. In some instances, the fetal support tissue comprises cells, substantially all of which are dead. In some instances, the fetal support tissue comprises umbilical cord amniotic membrane and at least a portion of Wharton's Jelly. In some instances, fetal support tissue is cryopreserved, lyophilized, sterilized, or a combination thereof. In some instances, the composition is a gel, a solution, or a suspension. In some instances, the composition is a gel. In some instances, the composition is a dry powder. In some instances, the composition is a powder that has been reconstituted in an isotonic solution. In some instances, the HC-HA/PTX3 is native HC-HA/PTX3, reconstituted HC-HA/PTX3, or a combination thereof. In some instances, the tissue having unwanted changes is ocular, cardiac, skin, joint, spine, soft tissue, cartilage, bone, tendon, ligament, nerve, intervertebral disc, spinal cord, brain, or muscle tissue. In some instances, the tissue is cardiac tissue. In some instances, the tissue is ocular tissue. In some instances, the tissue comprises degenerated tissue, a burn, a laceration, ischemic tissue, a wound, an injury, an ulcer, or a surgical incision. In some instances, the injury is a myocardial infarction. In some instances, the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell. In some instances, the methods further comprise contacting the fibroblastic cell with TGFβ1.

Disclosed herein, in some embodiments, are methods of treating cardiac tissue having unwanted changes due to myocardial infarction, comprising: contacting fibroblastic cells within the cardiac tissue during a stent placement surgical procedure with HC-HA/PTX3 for a period of time sufficient for abnormal fibroblastic cells to be reprogrammed to cardiomyocytes or cardiac progenitor cell that can differentiate to cardiomyocytes. In some embodiments, the HC-HA/PTX3 is comprised in a composition comprising: (a) a preparation comprising HC-HA/PTX3; and (b) a pharmaceutically acceptable diluent, excipient, vehicle, or carrier. In some instances, the tissue is not scar tissue. In some instances, the tissue is scar tissue. In some instances, the abnormal fibroblastic cells are generated by degenerative disease, aging, scarring, wound, burn, radiation, chemotherapy, surgical incision, laceration, ulceration, injury, or ischemia. In some instances, the fibroblastic cell is a fibroblast, a myofibroblast, a dermal fibroblast, a corneal fibroblast, or a cardiac fibroblast. In some instances, the fibroblastic cell is not a myofibroblast differentiated from an amniotic membrane stromal cell. In some instances, the myofibroblast is abnormally differentiated. In some instances, the myofibroblast is present in the tissue following damage or degeneration of the tissue. In some instances, the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof. In some instances, the fetal support tissue is selected from placenta, placental amniotic membrane, umbilical cord, umbilical cord amniotic membrane, chorion, amnion-chorion, amniotic stroma, amniotic jelly, or a combination thereof. In some instances, the fetal support tissue is frozen or previously frozen. In some instances, the fetal support tissue is substantially free of red blood cells. In some instances, the fetal support tissue comprises umbilical cord substantially free of a vein or artery. In some instances, the fetal support tissue comprises cells, substantially all of which are dead. In some instances, the fetal support tissue comprises umbilical cord amniotic membrane and at least a portion of Wharton's Jelly. In some instances, fetal support tissue is cryopreserved, lyophilized, sterilized, or a combination thereof. In some instances, the composition is a gel, a solution, or a suspension. In some instances, the composition is a gel. In some instances, the composition is a dry powder. In some instances, the composition is a powder that has been reconstituted in an isotonic solution. In some instances, the HC-HA/PTX3 is native HC-HA/PTX3, reconstituted HC-HA/PTX3, or a combination thereof. In some instances, the progenitor cell is a cardiovascular progenitor cell.

Disclosed herein, in some embodiments, are methods of treating a condition characterized by abnormal fibroblastic cell differentiation in a subject in need thereof comprising, contacting fibroblastic cells within a tissue affected by the condition in the subject with HC-HA/PTX3 for a period of time sufficient for the fibroblastic cells to be reprogrammed to progenitor cells or normal mesenchymal cells characteristic of the tissue. In some embodiments, the HC-HA/PTX3 is comprised in a composition comprising: (a) a preparation comprising HC-HA/PTX3; and (b) a pharmaceutically acceptable diluent, excipient, vehicle, or carrier. In some instances, the tissue is not scar tissue. In some instances, the tissue is scar tissue. In some instances, the abnormal fibroblastic cells are generated by degenerative disease, aging, scarring, wound, burn, radiation, chemotherapy, surgical incision, laceration, ulceration, injury, or ischemia. In some instances, the fibroblastic cell is a fibroblast, a myofibroblast, a dermal fibroblast, a corneal fibroblast, or a cardiac fibroblast. In some instances, the fibroblastic cell is not a myofibroblast differentiated from an amniotic membrane stromal cell. In some instances, the myofibroblast is abnormally differentiated. In some instances, the myofibroblast is present in the tissue following damage or degeneration of the tissue. In some instances, the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof. In some instances, the fetal support tissue is selected from placenta, placental amniotic membrane, umbilical cord, umbilical cord amniotic membrane, chorion, amnion-chorion, amniotic stroma, amniotic jelly, or a combination thereof. In some instances, the fetal support tissue is frozen or previously frozen. In some instances, the fetal support tissue is substantially free of red blood cells. In some instances, the fetal support tissue comprises umbilical cord substantially free of a vein or artery. In some instances, the fetal support tissue comprises cells, substantially all of which are dead. In some instances, the fetal support tissue comprises umbilical cord amniotic membrane and at least a portion of Wharton's Jelly. In some instances, fetal support tissue is cryopreserved, lyophilized, sterilized, or a combination thereof. In some instances, the composition is a gel, a solution, or a suspension. In some instances, the composition is a gel. In some instances, the composition is a dry powder. In some instances, the composition is a powder that has been reconstituted in an isotonic solution. In some instances, the HC-HA/PTX3 is native HC-HA/PTX3, reconstituted HC-HA/PTX3, or a combination thereof. In some instances, the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell.

Disclosed herein, in some embodiments, are in vitro methods of producing a progenitor cell, comprising: contacting a culture of fibroblastic cells with HC-HA/PTX3 for a time sufficient to reprogram the fibroblastic cells to a progenitor cells. In some embodiments, the HC-HA/PTX3 is comprised in a composition comprising: (a) a preparation comprising HC-HA/PTX3; and (b) a pharmaceutically acceptable diluent, excipient, vehicle, or carrier. In some instances, the preparation is an acellular extract of fetal support tissue, a cell culture matrix, purified HC-HA/PTX3, reconstituted HC-HA/PTX3, or a combination thereof. In some instances, the fetal support tissue is selected from placenta, placental amniotic membrane, umbilical cord, umbilical cord amniotic membrane, chorion, amnion-chorion, amniotic stroma, amniotic jelly, or a combination thereof. In some instances, the fetal support tissue is frozen or previously frozen. In some instances, the fetal support tissue is substantially free of red blood cells. In some instances, the fetal support tissue comprises umbilical cord substantially free of a vein or artery. In some instances, the fetal support tissue comprises umbilical cord amniotic membrane and at least a portion of Wharton's Jelly. In some instances, the fetal support tissue is cryopreserved, lyophilized, sterilized, or a combination thereof. In some instances, the HC-HA/PTX3 is native HC-HA/PTX3, reconstituted HC-HA/PTX3, or a combination thereof. In some instances, the fibroblastic cell is a fibroblast, a myofibroblast, a dermal fibroblast, a corneal fibroblast, or a cardiac fibroblast. In some instances, the fibroblast is a human corneal fibroblast. In some instances, the progenitor cell is a mesenchymal progenitor cell, a neural crest progenitor, a hematopoietic progenitor cell, a mammary progenitor cell, an intestinal progenitor cell, an endothelial progenitor cell, a neural progenitor cell, an olfactory progenitor cell, a testicular progenitor cell, or a cardiovascular progenitor cell. In some instances, the methods further comprise contacting the fibroblastic cell with TGFβ1.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the disclosure will be obtained by reference to the following detailed description that sets forth illustrative embodiments, in which the principles of the disclosure are utilized, and the accompanying drawings of which:

FIGS. 1A-1C illustrate HC-HA/PTX3 but not HA, promotes significant aggregation, suppresses canonical TGFβ signaling and myofibroblast differentiation. FIG. 1A illustrates P3 HCF (5,000 cells per 96-well) were cultured in DMEM+10% FBS on plastic with or without immobilized HA or HC-HA/PTX3 (each at 2 μg of HA per 96-well) for 72 h and then treated with or without TGFβ1 for 24 h and 72 h before being harvested for mRNA quantification (FIG. 1B) or immunostaining of pSMAD2/3 and α-SMA (FIG. 1C). For TGF-β1 ELISA, the cells were treated with or without TGF-β1 (10 ng/ml) for 24 h and then cultured in the fresh medium for another 24 h. The supernatants were collected for TGFβ1 ELISA. For TGFβ2 and TGFβ3 ELISA, the cells were treated without or without TGFβ1 (10 ng/ml) for 48 h. * or ^{#}P<0.05, **P<0.01 when compared to their corresponding plastic controls. N=3. Bar=100 μm

FIGS. 2A-2C illustrate HC-HA/PTX3 promoted HCF into keratocytes without TGFβ1 but into neural crest progenitors with TGFβ1. P3 HCF were seeded on plastics with or without immobilized HA, HC-HA/PTX3 complex for 72 h, and then treated with or without TGFβ1 for 24 h before being harvested for mRNA quantification of keratocyte markers such as keratocan, NC markers such as p75NTR, HNK1, Sox9, KLF4, Snail1, and MSX1 using the expression level on plastic without TGFβ1 as 1 (FIG. 2A), and for immunostaining of p75NTR (FIG. 2C). For determination of protein of keratocan and p75NTR (FIG. 2B), the cells were treated with or without TGF-β1 for 48 h using β-actin as the loading control. * P<0.05, **P<0.01, ***P<0.001 when compared to their corresponding plastic controls. N=3. Bar=25 μm.

FIGS. 3A-3C illustrate that the induced NC progenitors differentiate into corneal endothelial cells. P3 HCF were seeded on plastics with or without immobilized HA, HC-HA/PTX3 complex for 72 h, and then treated with or without TGFβ1 for 24 h before being harvested for mRNA quantitation of HCEC and stromal markers. FIG. 3A illustrates mRNA expression of several endothelial markers in native HCEC, HCF, neural crest (NC) like cells, and induced HCEC (iHCEC). FIG. 3B illustrates mRNA expression of HCF fibroblastic markers, vimentin and CD34, in native HCEC, HCF, neural crest (NC) like cells, and induced HCEC (iHCEC). * P<0.05, **P<0.01, ***P<0.001 when compared to their corresponding plastic controls. N=3. Bar=25 μm. For induction of HCEC, HCF were cultured on HC-HA/PTX3 complex in serum-free DMEM-ITS with or without challenge of TGFβ1 for 3 days and further cultured in low-calcium DMEM with 10% FBS to induce corneal endothelial like cells for 3 weeks. The staining pattern of endothelial markers Na—K-ATPase, α-catenin, β-catenin, F-actin, N-cadherin, p120, ZO-1 and fibroblastic markers S100A4 were compared among native HCEC, iHCEC and HCF (FIG. 3C).

FIGS. 4A-4G illustrate suppression of canonical TGFβ signaling is mediated via downregulation of TGFβRII, which is linked to upregulation and nuclear translocation of cyclin D1. P3 HCF were seeded on plastic with or without immobilized HA, HC-HA/PTX3 complex for 72 h, and then treated with or without TGFβ1±Cyclin D1 siRNA for 24 h before being harvested for mRNA quantitation of TGFβRI, TGFβRII, and TGFβRIII, Cyclin D1, and NC markers (FIGS. 4A, 4D, 4E, and 4G), for immunostaining of pSMAD2/3, α-SMA, Cyclin D1 and p75NTR, for 48 h for protein quantitation of TGFβRI, TGFβRII, Cyclin D1, and p75NTR using β-actin as the loading control (FIGS. 4B and 4F). For some experiments, Cyclin D1 siRNA was added (FIGS. 4C, 4D, 4E, 4F, and 4G). * or #P<0.05. **P<0.01 and ***P<0.001. N=3. Bar=25 μm.

FIGS. 5A-5C illustrate nuclear cyclin D1 was temporally associated with upstream nuclear CD44ICD, TAK1, and JNK1. P3 HCF were seeded on glass in DMEM+10% FBS for 24 h, then in DMEM+ITS for 24 h, treated with/without PBS or HA or HC-HA/PTX3±TGFβ1 (10 ng/ml)±Marimastat (10 μM) or ±DAPT (10 μM) or ±both for 0, 5, 15, 30 and 45 minutes before being harvested for immunostaining of CD44-ICD, TAK1, JNK1, and Cyclin D1 (FIG. 5A) and for 5 minutes before being harvested for Western blotting of cytoplasmic and nuclear CD44-ICD after compartmental separation of the cellular components, active MT1-MMP and active γ-secretase (FIG. 5B). FIG. 5C illustrates mRNA expression of these markers.

FIGS. 6A-6B illustrate that nuclear CD44ICD was regulated by activation of MT1-MMP and γ-Secretase. P3 HCF were seeded on glass in DMEM+10% FBS for 24 hours, then in DMEM+ITS for 24 h, treated with/without PBS or HA±TGFβ1 or HC-HA/PTX3±TGFβ1 (10 ng/ml) for 5 minutes before being harvested for immune-precipitation by CD44 antibody (FIG. 6B), and Western blotting by active MT1-MMP and active γ-secretase antibodies (FIG. 6A). β-actin was used as the loading control.

FIGS. 7A-7D show that human corneal myofibroblasts formed aggregates and be reversed to keratocytes by HC-HA/PTX3. HCF were cultured at the density of 5000 cells/96-well in DMEM+10% FBS for 3 days. The cells were starved for 1 day and then treated with 10 ng/ml TGFβ1 for 3 days to induce myofibroblasts. The induced myofibroblasts were verified by immunostaining of α-SMA (FIG. 7A). The myofibroblasts were passaged and further cultured on plastic or HA or HC-HA/PTX3 for up to 7 days. After passage, the cells formed aggregates at day 1 and retained some aggregates at day 4 on HC-HA/PTX3 but not plastic or HA (FIG. 7D) and then all the cells were expanded to a single layer of stromal cells in 7 days (FIG. 7B). At day 1, mRNA and protein expression of keratocan was significantly elevated (B and C, *p<0.05, ***p<0.001, n=3) while expression of α-SMA was significantly reduced in the cells on HC-HA/PTX3, but not those on plastic or HA (FIG. 7D). At day 4 and day 7, the myofibroblasts on plastic or HA retained their myofibroblast characteristic staining of α-SMA, but not on HC-HA/PTX3 (FIG. 7D). Interestingly, HC-HA/PTX3, not plastic or HA, promoted mRNA and protein expression of keratocytes (FIG. 7B and FIG. 7C). Bar=100 μm.

FIGS. 8A-8F illustrate that HCF can also form aggregates, be reversed to keratocytes and resist to TGFβ1 on HC-HA/PTX3. HCF were cultured at the density of 5000 cells/96-well in DMEM+10% FBS on plastic or HA or HC-HA/PTX3 for up to 7 days. After passage, all the cells formed aggregates on HC-HA/PTX3 but only a few on plastic or HA at day 1. The cells on HC-HA/PTX3 but not on plastic or HA retained aggregates until day 7 (FIG. 8A). At day 1, mRNA and protein expression of keratocan was significantly elevated (FIGS. 8B and 8C, *p<0.05, ***p<0.001, n=3). Expression of TGFβs and TGFβRs were not promoted by HC-HA/PTX3 except TGFβ3, an anti-TGFβ format (FIG. 8D). pSMAD2/3 retained in cytoplasm in HCF on HC-HA/PTX3 (FIG. 8E). Under challenge of TGFβ1, the cells on plastic or HA, but not on HC-HA/PTX3 expressed α-SMA (FIG. 8F). Bar=100 μm.

FIGS. 9A-9D illustrate that the reversal to keratocytes is mediated by canonical BMP signaling. The fibroblasts were cultured on plastic or HA or HC-HA/PTX at the density of 5,000 cells/96-well on plastic, HA or HC-HA/PTX3 in DMEM+10% FBS for 24 h for real-time PCR and immunostaining, for 48 h for Western blotting. The mRNAs were extracted and the levels of BMPs, BMPRs and keratocan were determined by real-time PCR (FIG. 9A and FIG. 9C, *p<0.05, **p<0.01, ***p<0.001, n=3). Immunostaining was performed for cytolocation of pSMAD1/5 (FIG. 9B). Western blotting was performed for expression of keratocan protein (FIG. 9D). Bar=100 μm.

FIGS. 10A-10F illustrate aggregation mediated by SDF1-CXCR4 signaling regulates BMP signaling and reversal to keratocytes. The fibroblasts were cultured on plastic or HA or HC-HA/PTX with or without CXCR4 inhibitor AMD3100 at the density of 5,000 cells/96-well on plastic, HA or HC-HA/PTX3 in DMEM+10% FBS for 24 h for real-time PCR and immunostaining, for 48 h for Western blotting. Fibroblasts were visualized on day 1, day 4, and day 7 (FIG. 10A). The mRNAs were extracted and the levels of SDF1, CXCR4, BMPs and BMPRs were determined by real-time PCR (FIG. 10B and FIG. 10D, *p<0.05, **p<0.01, ***p<0.001, n=3). Immunostaining was performed for cytolocation of CXCR4 and pSMAD1/5 (FIG. 10C and FIG. 10E). Western blotting was performed for protein quantitation of CXCR4 and keratocan (FIG. 10F). Bar=100 μm.

FIGS. 11A-11B illustrate sequential activation of SDF1/CXCR4 and BMP signaling. P3 HCF were seeded on plastic in DMEM+10% FBS and treated with PBS or HA or HC-HA/PTX3 for 0, 5, 15, 30, 45, 60 minutes, 24 and 48 hours before being harvested for real-time PCR of SDF1, CXCR4, BMP4 and BMP6 (FIG. 11A), and for immunostaining of CXCR4 and pSMAD1/5 (FIG. 11B). N=3, Bar=100 μm.

FIGS. 12A-12B illustrate inhibition of SDF1/CXCR4 signaling aborts aggregation and BMP signaling. P3 HCF were seeded on plastic in DMEM+10% FBS and treated with PBS or HA or HC-HA/PTX3 with or without CXCR4 inhibitor AMD3100 for 0, 5, 15, 30, 45, 60 minutes, 24 and 48 hours before being harvested for real-time PCR of SDF1, CXCR4, BMP4 and BMP6 (FIG. 12A), and for immunostaining of CXCR4 and pSMAD1/5 (FIG. 12B). N=3. Bar=100 μm.

FIGS. 13A-13B illustrate inhibition of BMP signaling does not affect SDF1-CXCR4 signaling and aggregation. P3 HCF were seeded on plastic in DMEM+10% FBS and treated with PBS or HA or HC-HA/PTX3 with or without BMP inhibitor SB431542 for 0, 5, 15, 30, 45, 60 minutes, 24 and 48 hours before being harvested for real-time PCR of SDF1, CXCR4, BMP4 and BMP6 (FIG. 13A), and for immunostaining of CXCR4 and pSMAD1/5 (FIG. 13B). N=3. Bar=100 μm.

FIGS. 14A-14B illustrate progressive loss of nuclear Pax6 neural crest progenitor status in LNC after serial passage. P10 LNC were seeded at 1×10⁵/ml per 96 well with 5% coated MG in Modified Embryonic Stem Cell Medium (MESCM). Changes of cell phenotype by serial passage were determined by quantitative RT-PCR for mRNA levels of neural crest markers such as Pax6, Sox2, p75NTR, Musashi-1, and Nestin in P10 LNC using the expression level at passage 2 (P2) set as 1 (FIG. 14A, ##p<0.01, n=3; bars from left to right for each gene represent mRNA levels in cells at P2, P4, P6, P8, and P10) and by immunofluorescence staining of Pax6, Sox2, p75NTR, Musashi-1, and Nestin between P4 and P10 LNC (FIG. 14B, Bar=100 μm).

FIGS. 15A-15D illustrate immobilized HC-HA/PTX3, but not 3D Matrigel™, reverted P10 LNC to nuclear Pax6+ neural crest progenitors. P10 LNC were seeded at 1×10⁵/mL per 96 well with 5% coated MG, 3D MG, or immobilized HC-HA/PTX3 in Modified Embryonic Stem Cell Medium (MESCM). Phase contrast microscopy was used to monitor the sphere formation at 24 h and 48 h. (FIG. 15A, Bar=50 μm). Phenotypic characterization was performed by quantitative RT-PCR to compare the mRNA levels of Pax6, p75NTR, Musashi-1, Nestin, Msx-1, and FoxD3 in HC-HA/PTX3 against coated MG set as 1 (FIG. 15B, ** p<0.01, n=3) or against expression levels in 3D MG (FIG. 15B, ##p<0.01, n=3). Immunofluorescence staining to Pax6, Sox2, p75NTR and Musashi-1 (FIG. 15C, nuclear counterstaining by Hoechst 33342, bars=25 μm). Cell aggregates derived from coated MG, 3D MG and HC-HA/PTX3 were rendered single cells and subjected to different differentiation induction media before being assessed by immunofluorescence to neurofilament M (NFM), 04, and glial fibrillary acidic protein (GFAP) (FIG. 15D, nuclear counterstaining by Hoechst 33342, Bars=50 μm).

FIGS. 16A-16C illustrate soluble HC-HA/PTX3 also promoted early cell aggregation and nuclear Pax6+ neural crest progenitors in P10 LNC. P10 LNC were seeded at 1×10⁵/mL per 96 well coated with 3D MG or immobilized HC-HA/PTX3 or coated MG where soluble HC-HA/PTX3 added at 25 μg/mL in MESCM. Cell morphology and aggregation (marked by a white arrow) were assessed by phase contrast microscopy (FIG. 16A, bar=100 μm). Quantitative RT-PCR at different time points to compare the mRNA level of p75NTR, NGF and Musashi-1 in soluble HC-HA/PTX3 using the expression level at time 0 set as 1 (FIG. 16C, ##p<0.01, n=3). Resultant cell phenotype was characterized by immunofluorescence staining to Pax6, Sox2 and p75NTR at 48 h. (FIG. 16B, nuclear counterstaining by Hoechst 33342, Bar=50 μm)

FIGS. 17A-17D illustrates cell aggregation and nuclear Pax6 expression promoted by soluble HC-HA/PTX3 was mediated by CXCR4/SDF-1 signaling. P10 LNC were seeded at 1×10⁵/mL per 96 well on 3D MG or coated MG with addition of 25 μg/mL soluble HC-HA/PTX3, of which the latter was added with 0.1% DMSO with or without 20 μg/mL AMD3100 in MESCM. Cell aggregation was assessed by phase contrast microscopy (FIG. 17A, Bar=100 μm). CXCR4/SDF-1 signaling was determined by quantitative RT-PCR to compare the mRNA transcript levels of SDF-1 and CXCR4 in HC-HA/PTX3 or HC-HA/PTX3+AMD3100 against the expression level on 3D Matrigel at time 0 set as 1, respectively. (FIG. 17B, ** p<0.01 or ##p<0.01, n=3) Phenotypic characterization of resultant cells was performed by quantitative RT-PCR for the mRNA transcript levels of Pax6, p75NTR, NGF, Musashi-1, Msx-1, and FoxD3 were compared between HC-HA/PTX3 or HC-HA/PTX3+AMD3100 using the expression level of coated MG set as 1 (FIG. 17C, ** p<0.01) and by immunofluorescence staining of CXCR4, SDF-1, and Pax6 (FIG. 17D, nuclear counterstaining by Hoechst 33342, Bar=50 μm).

FIGS. 18A-18D illustrate CXCR4/SDF-1 was required for activation of BMP signaling in P10 LNC by soluble HC-HA/PTX3. P10 LNC single cells were seeded at 1×10⁵/mL per 96 well in 3D MG or coated MG with 25 μg/mL soluble HC-HA/PTX3, of which the latter was added with or without AMD3100 in MESCM. Quantitative RT-PCR of BMP ligands and BMP receptors were compared transcription levels of P4 and P10 LNC in soluble HC-HA/PTX3 using the expression level of P4 LNC set as 1 (FIG. 18A, ** P<0.01, n=3) Immunofluorescence staining confirmed nuclear staining pSmad1/5/8 (red) of early P4 and late P10 LNC on coated MG. (FIG. 18B, nuclear counterstaining by Hoechst 33342, bar=25 μm) Quantitative RT-PCR at different time points was used to compare the mRNA expression level of BMP ligands in soluble HC-HA/PTX3 (FIG. 18C, ** p<0.01, n=3) against HC-HA/PTX3+AMD3100 (FIG. 18C, ##P<0.01, n=3) using the expression level of 3D MG at time 0 set as 1. Immunofluorescence staining of pSmad1/5/8 (positive nuclear staining marked by white arrowheads) was also compared (FIG. 18D, nuclear counterstaining by Hoechst 33342, bar=25 μm).

FIG. 19A-19E illustrate cell aggregation and CSCR4/SDF-1 signaling promoted by HC-HA/PTX3 was not affected by BMP signaling. P10 LNC on coated MG in MESCM were pre-treated with without LDN-193189 or transfected with siRNAs for BMPR1A, BMPR1B, BMPR2 and ACVR1 before being seeded on coated MG with or without soluble HC-HA/PTX3 in MESCM. The transfection efficiency was verified by qRT-PCR when compared to scrambled RNA (scRNA) as the control (FIG. 19A, ** p<0.01, n=3). BMP signaling was measured by immunofluorescence staining to pSmad1/5/8 (FIG. 19B) and cell aggregation was detected by phase contrast microscopy (FIG. 19C, bar=100 μm). CXCR4/SDF-1 signaling was assessed by qRT-PCR for the expression of CXCR4 and SDF-1 transcripts using the expression level by cells with HC-HA/PTX3+scRNA at time 0 set as 1. (FIG. 19D, * p>0.05, n=3) and by immunofluorescence staining to CXCR4 and Pax6 (FIG. 19E, nuclear counterstaining by Hoechst 33342, bar=25 μm).

FIG. 20 illustrates an example cellular differentiation pathway, with cell type represented in boxes and example of markers of cell type indicates above or below each cell type.

### DETAILED DESCRIPTION OF THE DISCLOSURE

Provided herein, in certain embodiments, are uses of HC-HA/PTX3, including preparations or compositions comprising HC-HA/PTX3, to reprogram the cellular phenotype of a cell into a different cellular phenotype. Such reprogramming is used in methods provided herein of, for example, reversing a diseased or damaged state of a tissue (e.g., a damaged or scarred tissue, or a tissue affected by a disease such as a degenerative disease); reprogramming a differentiated cell in a tissue to a progenitor cell, thereby rejuvenating the tissue; reprogramming a first phenotype of a cell in a tissue to a progenitor cell, and differentiating the progenitor cell into a second phenotype, thereby regenerating the tissue. Also provided herein are uses of HC-HA/PTX3, including preparations or compositions comprising HC-HA/PTX3, in compositions with therapeutic cells.

Provided herein, in certain embodiments, are methods of reprogramming a first phenotype of a cell to a second phenotype. In some embodiments, the method comprises contacting the cell with HC-HA/PTX3 for a time sufficient to reprogram the first phenotype of the cell to the second phenotype. In some embodiments, the first cellular phenotype is a phenotype of differentiated cell. In some embodiments, the second cellular phenotype is a phenotype of a progenitor cell. In some embodiments, the reprogrammed cell is within a tissue. In some embodiments the cell reprogrammed to a second phenotype is differentiated into a differentiated cell type corresponding to the tissue in which it is contained. Such methods may be used in vivo to rejuvenate or regenerate tissue that is damaged, degenerated, scarred, affected by a disease, or aged.

Further provided herein, in certain embodiments, are methods of treating a condition characterized by unwanted fibroblastic cell differentiation in a subject in need thereof. The method can comprise contacting a fibroblastic cell within a tissue affected by the condition in the subject with HC-HA/PTX3 for a period of time sufficient to reprogram a phenotype of the fibroblastic cell to a different phenotype, thereby treating the condition.

Further provided herein, in certain embodiments, are in vitro methods of producing a progenitor cell, comprising: contacting a culture of fibroblastic cells or other differentiated cells with HC-HA/PTX3 for a time sufficient to reprogram the fibroblastic cells to progenitor cells. Such progenitor cells may be differentiated in to a differentiated cell type of interest. Such methods may be employed in tissue engineering for generating tissue or organs for use in transplantation surgery.

In some embodiments, the methods provide an improved treatment for tissue having unwanted changes due to degeneration from a disease, aging or scarring, or following an insult, such as a burn, wound, injury, ulcer, radiation, chemotherapy, or surgery by contacting the tissue with a preparation comprising HC-HA/PTX3 within a window of time that allows for cellular reprogramming to occur. In some embodiments, the methods provide a prophylactic treatment for tissue anticipated to receive unwanted changes due to degeneration from a disease, aging or scarring, or following an insult, such as a burn, wound, injury, ulcer, radiation, chemotherapy, or surgery by contacting the tissue with a preparation comprising HC-HA/PTX3. In some embodiments, the unwanted change is a differentiation of a cell of the tissue from a first cell into a second cell. In some embodiments, the second cell is a harmful cell or a potentially harmful cell. One example of an unwanted change is the differentiation of a fibroblast in a cardiac tissue into a myofibroblast following a myocardial infarction. In some embodiments, myofibroblasts are involved in the wound healing process. However, in some cases, the prolonged presence of myofibroblasts in injured tissue results in unwanted changes, for example cardiac fibrosis in cardiac tissue.

As used herein “phenotype” when used in reference to a cell or “cellular phenotype” refers to the molecular or cellular characteristics, properties, and/or function of the cell. In some embodiments, the cellular phenotype is defined by one or more of a cell aggregation characteristic, cell shape, or expression of at least one cell-specific marker. In some embodiments, the cellular phenotype corresponds to a phenotype of a progenitor cell. In some embodiments, the progenitor cell phenotype refers to a cell that is capable of differentiating into one or more different types of differentiated cells. In some embodiments, the progenitor cell phenotype corresponds to the cellular phenotype of a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell. In some embodiments, the cellular phenotype corresponds to a phenotype of differentiated cell. In some embodiments, the differentiated cellular phenotype corresponds to the phenotype of a nerve cell, a bone cell, an epithelial cell, a liver cell, kidney cell, a pancreatic cell, a lung cell, a muscle cell, a smooth muscle cell, a cardiac muscle cell, a corneal cell, an epithelial cell, a skin cell, a limbal niche cell, fibroblast, keratocyte, endothelial cell, or myofibroblast. In some embodiments, the differentiated cellular phenotype corresponds to the phenotype of a cell within a tissue such as ocular, cardiac, skin, joint, spine, soft tissue, cartilage, bone, tendon, ligament, nerve, intervertebral disc, spinal cord, brain, or muscle tissue.

In some embodiments, a cell has a first phenotype. In some embodiments, the methods described herein can comprises contacting a cell having a first phenotype with HC-HA/PTX3 or a preparation or composition comprising HC-HA/PTX3 for a time sufficient to reprogram the first phenotype of the cell to a second phenotype. In some embodiments, a first phenotype or a second phenotype is characterized by a cell aggregation characteristic, cell shape, or expression of at least one cell-specific marker. In some embodiments, the cell aggregation characteristic is selected from aggregation and no aggregation of cells. In some embodiments, the cell shape is selected from spindle and round.

In some embodiments the phenotype is characterized by expression (or lack of expression) of a cell-specific marker. In some embodiments, the cell-specific marker is a neural crest cell marker. In some embodiments, the neural crest cell marker is Pax6, p75NTR, Musashi-1, Sox2, Nestin, Sox9, FOXD3, MSX1, HNK1, Snail1/2, Twist1/2, AP2α, AP2β, or a combination thereof. In some embodiments, the cell-specific marker is an endothelial cell marker. In some embodiments, the endothelial cell specific marker is Na—K ATPase, ZO1, N-cad, or a combination thereof. In some embodiments, the cell-specific marker is a keratocyte cell marker. In some embodiments, the keratocyte cell marker is keratocan, CD34, ALDH3A1, PTDGS, or a combination thereof. In some embodiments, the cell-specific marker is a fibroblast cell marker. In some embodiments, the fibroblast cell marker is integrin α5β1, fibronectin, EDA, or a combination thereof. In some embodiments, the cell-specific marker is a myofibroblast cell marker. In some embodiments, the myofibroblast cell marker is α-SMA, S100A4, or a combination thereof. In some embodiments, the time sufficient to reprogram the first phenotype of the cell to a second phenotype is at least 1 hour, 2 hours, 3 hours, 4 hours, 5 hours, 6 hours, 7 hours, 8 hours, 9 hours, 10 hours, 11 hours, 12 hours, 24 hours, 36 hours, 2 days, 3 days, 4 days, 5 days, 6 days, 7 days, 2 weeks, 3 weeks, or 4 weeks. In some embodiments, the time sufficient to reprogram the first phenotype of the cell to a second phenotype is less than 1 hour, 2 hours, 3 hours, 4 hours, 5 hours, 6 hours, 7 hours, 8 hours, 9 hours, 10 hours, 11 hours, 12 hours, 24 hours, 36 hours, 2 days, 3 days, 4 days, 5 days, 6 days, 7 days, 2 weeks, 3 weeks, or 4 weeks. In some embodiments, the first phenotype is a differentiated cell phenotype. In some embodiments, the second phenotype is a progenitor cell phenotype.

In some embodiments, the first phenotype comprises a phenotype of a first cell. In some embodiments, the first cell is a differentiated cell. In some embodiments, the first cell is selected from a limbal niche cell, fibroblast, keratocyte, endothelial cell, or myofibroblast. In some embodiments, the first phenotype comprises no cell aggregation. In some embodiments, the first phenotype comprises a cell shape of spindle. In some embodiments, the first phenotype comprises expression of at least one cell-specific marker. In some embodiments, the cell-specific marker characterizing the first phenotype is a limbal niche cell marker, a fibroblast cell marker, a keratocyte cell marker, an endothelial cell marker, or a myofibroblast cell marker.

In some embodiments, the second phenotype comprises a phenotype of a second cell. In some embodiments, the second cell is a progenitor cell. In some embodiments, the second cell is selected from a neural crest progenitor cell, limbal niche cell, fibroblast, keratocyte, or endothelial cell. In some embodiments, the second phenotype comprises cell aggregation. In some embodiments, the second phenotype comprises a cell shape of round. In some embodiments, the first phenotype comprises expression of at least one-specific marker. In some embodiments, the cell-specific marker characterizing the second phenotype is a neural crest cell marker, limbal niche cell marker, a fibroblast cell marker, a keratocyte cell marker, or an endothelial cell marker.

In some embodiments, the methods described herein further comprising detecting the first phenotype, the second phenotype, or the combination thereof. In some embodiments, the methods described herein further comprising detecting a cell-specific marker characterizing the first phenotype, a cell-specific marker characterizing the second phenotype, or the combination thereof.

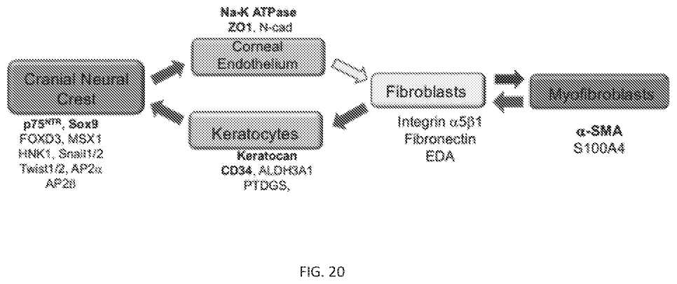

In some embodiments, the contacting prevents differentiation of a first cell into a second cell (e.g. Example 1, describing prevention of a fibroblast from differentiating into a myofibroblast). In some embodiments, the second cell is produced as a result of an insult, such as a burn, wound, laceration, injury, ulcer, radiation, chemotherapy, surgery, or due to ischemia. In some embodiments, the contacting reprograms a cell into an earlier cell from the same cellular differentiation lineage (e.g. Example 2, describing reprograming of a fibroblast into a keratocyte-like progenitor). In some embodiments, a cellular differentiation lineage comprises a progenitor cell and any cell that differentiates from (a) the progenitor cell or (b) a cell differentiated from a cell that differentiated from the progenitor cell, and so forth. In some embodiments, an example of a cellular differentiation lineage is illustrated in FIG. 20.

In some embodiments, a cell is a myofibroblast and an earlier cell is a fibroblast, keratocyte, endothelial cell, or neural crest progenitor. In some embodiments, a cell is a fibroblast and an earlier cell is a keratocyte, endothelial cell, or neural crest progenitor. In some embodiments, a cell is a keratocyte and an earlier cell is a neural crest progenitor. In some embodiments, a cell is an endothelial cell and an earlier cell is a neural crest progenitor. In some embodiments, a cell is a limbal niche cell and an earlier cell is a neural crest progenitor.

### Certain Definitions

Unless defined otherwise, all technical and scientific terms used herein have the same meaning as is commonly understood by one of skill in the art to which the claimed subject matter belongs.

As used herein, ranges and amounts can be expressed as “about” a particular value or range. About also includes the exact amount. Hence “about 5 μg” means “about 5 μg” and also “5 μg.” Generally, the term “about” includes an amount that would be expected to be within experimental error. In some embodiments, “about” refers to the number or value recited, “+” or “−” 20%, 10%, or 5% of the number or value.

As used herein, “fetal support tissue product” means any isolated product derived from tissue used to support the development of a fetus. Examples of fetal support tissue include, but are not limited to, (i) placental amniotic membrane (PAM), or substantially isolated PAM, (ii) umbilical cord amniotic membrane (UCAM) or substantially isolated UCAM, (iii) chorion or substantially isolated chorion, (iv) amnion-chorion or substantially isolated amnion-chorion, (v) placenta or substantially isolated placenta, (vi) umbilical cord or substantially isolated umbilical cord, or (vii) any combinations thereof. In some embodiments, the fetal support tissue is selected from the group consisting of placental amniotic membrane (PAM), umbilical cord amniotic membrane (UCAM), chorion, amnion-chorion, placenta, umbilical cord, and any combinations thereof. In some embodiments, the fetal support tissue comprises umbilical cord. In some embodiments, the fetal support tissue comprises placental amniotic membrane and umbilical cord. Fetal support tissue products include any form of the fetal support tissue, including cryopreserved, terminally-sterilized, lyophilized fetal support tissue, or powders resulting from grinding fetal support tissue. In some embodiments, the fetal support tissue product is ground, pulverized, morselized, a graft, a sheet, a powder, a gel, a homogenate, an extract, or a terminally-sterilized product.

As used herein, “placenta” refers to the organ that connects a developing fetus to the maternal uterine wall to allow nutrient uptake, waste elimination, and gas exchange via the maternal blood supply. The placenta is composed of three layers. The innermost placental layer surrounding the fetus is called amnion. The allantois is the middle layer of the placenta (derived from the embryonic hindgut); blood vessels originating from the umbilicus traverse this membrane. The outermost layer of the placenta, the chorion, comes into contact with the endometrium. The chorion and allantois fuse to form the chorioallantoic membrane.

As used herein, “chorion” refers to the membrane formed by extraembryonic mesoderm and the two layers of trophoblast. The chorion consists of two layers: an outer formed by the trophoblast, and an inner formed by the somatic mesoderm; the amnion is in contact with the latter. The trophoblast is made up of an internal layer of cubical or prismatic cells, the cytotrophoblast or layer of Langhans, and an external layer of richly nucleated protoplasm devoid of cell boundaries, the syncytiotrophoblast. The avascular amnion is adherent to the inner layer of the chorion.

As used herein, “amnion-chorion” refers to a product comprising amnion and chorion. In some embodiments, the amnion and the chorion are not separated (i.e., the amnion is naturally adherent to the inner layer of the chorion). In some embodiments, the amnion is initially separated from the chorion and later combined with the chorion during processing.

As used herein, “umbilical cord” refers to the organ that connects a developing fetus to the placenta. The umbilical cord is composed of Wharton's jelly, a gelatinous substance made largely from mucopolysaccharides. It contains one vein, which carries oxygenated, nutrient-rich blood to the fetus, and two arteries that carry deoxygenated, nutrient-depleted blood away. In some embodiments, the umbilical cord substantially lacks the vein and arteries. In some embodiments, the umbilical cord comprises all or a portion of Wharton's jelly.

As used herein, “placental amniotic membrane” (PAM) refers to amniotic membrane derived from the placenta. In some embodiments, the PAM is substantially isolated.

As used herein, “umbilical cord amniotic membrane” (UCAM) means amniotic membrane derived from the umbilical cord. UCAM is a translucent membrane. The UCAM has multiple layers: an epithelial layer; a basement membrane; a compact layer; a fibroblast layer; and a spongy layer. The UCAM lacks blood vessels or a direct blood supply. In some embodiments, the UCAM comprises Wharton's Jelly. In some embodiments, the UCAM comprises blood vessels and/or arteries. In some embodiments, the UCAM comprises Wharton's Jelly and blood vessels and/or arteries.

As used herein, “human tissue” means any tissue derived from a human body. In some embodiments, the human tissue is a fetal support tissue selected from the group consisting of placental amniotic membrane, umbilical cord, umbilical cord amniotic membrane, chorion, amnion-chorion, placenta, or any combination thereof.

As used herein, “minimal manipulation” means: (1) for structural tissue, processing that does not alter the original relevant characteristics of the tissue relating to the tissue's utility for reconstruction, repair, or replacement; and (2) for cells or nonstructural tissues, processing that does not alter the relevant biological characteristics of cells or tissues.

As used herein, “graft” means a matrix of proteins (e.g., collagen and elastin) and glycans (e.g., dermatan, hyaluronan, and chondroitin) that is used to replace damaged, compromised, or missing tissue. In certain instances, the matrix is laid down and host cells gradually integrate into the matrix.

As used herein, “sheet” means any continuous expanse or surface. In some embodiments, a sheet of a fetal support tissue product is substantially flattened. In some embodiments, a sheet of a fetal support tissue product is flat. In some embodiments, a sheet of fetal support tissue product is tubular. In some embodiments, the sheet is any shape or size suitable for the wound to be treated. In some embodiments, the sheet is a square, circle, triangle, or rectangle.

The term “fresh fetal support tissue” refers to fetal support tissue that is less than 10 days old following birth, and which is in substantially the same form as it was following birth.

“Substantially isolated” or “isolated,” when used in the context of a fetal support tissue, means that the fetal support tissue is separated from most other non-fetal support tissue materials (e.g., other tissues, red blood cells, veins, arteries) derived from the original source organism.

As used herein, the phrase “wherein the biological and structural integrity of the isolated fetal support tissue product is substantially preserved” means that when compared to the biological activity and structural integrity of fresh fetal support tissue, the biological activity and structural integrity of the isolated fetal support tissue has only decreased by about 5%, about 10%, about 15%, about 20%, about 25%, about 30%, about 35%, about 40%, about 50%, or about 60%.

As used herein, “processing” means any activity performed on a fetal support tissue or a preparation comprising HC-HA/PTX3, other than, recovery, donor screening, donor testing, storage, labeling, packaging, or distribution, such as testing for microorganisms, preparation, sterilization, steps to inactivate or remove adventitious agents, preservation for storage, and removal from storage.

As used herein, the terms “purified” and “isolated” mean a material (e.g., HC-HA/PTX3 complex) substantially or essentially free from components that normally accompany it in its native state. In some embodiments, “purified” or “isolated” mean a material (e.g., HC-HA/PTX3 complex) that is about 50% or more free from components that normally accompany it in its native state, for example, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% free from components that normally accompany it in its native state.

As used herein, “biological activity” means the activity of polypeptides and polysaccharides in the preparation comprising HC-HA/PTX3. In some embodiments, the biological activity of polypeptides and polysaccharides found in the preparation is anti-inflammatory, anti-scarring, anti-angiogenic, or anti-adhesion. In some embodiments, the biological activity refers to the in vivo activities of the HC-HA/PTX3 complex in the preparation or physiological responses that result upon in vivo administration of the preparation. In some embodiments, the biological activity of HC-HA/PTX3 complex in the fetal support tissue is substantially preserved. In some embodiments, the activity of polypeptides and polysaccharides found in the preparation promotes wound healing. In some embodiments, the activity of polypeptides and polysaccharides found in the preparation prevents scarring. In some embodiments, the activity of polypeptides and polysaccharides found in the preparation reduces inflammation. Biological activity, thus, encompasses therapeutic effects and pharmaceutical activity of the HC-HA/PTX3 complex in the preparation.

As used herein, “structural integrity” means the integrity of stroma and basement membrane that make up the fetal support tissue product. In some embodiments, the structural integrity of the fetal support tissue product results in suture pull out strength.

As used herein, a reconstituted HC-HA/PTX3 (rcHC-HA/PTX3) complex is an HC-HA/PTX3 complex that is formed by assembly of the component molecules of the complex in vitro. The process of assembling the rcHC-HA/PTX3 includes reconstitution with purified native proteins or molecules from biological sources, recombinant proteins generated by recombinant methods, or synthesis of molecules by in vitro synthesis. In some instances, the purified native proteins used for assembly of the rcHC-HA/PTX3 are proteins in a complex with other proteins (i.e. a multimer, a multichain protein or other complex). In some instances, PTX3 is purified as a multimer (e.g. a homomultimer) from a cell and employed for assembly of the rcHC-HA/PTX3 complex. In some embodiments, the rcHC-HA/PTX3 complex comprises HC1, HC2, HA, and PTX3. In some embodiments, the rcHC-HA/PTX3 complex comprises HC1, HC2, HA, PTX3 and TSG-6.

As used herein, a purified native HC-HA/PTX3 (nHC-HA/PTX3) complex refers to an HC-HA/PTX3 complex that is purified from a biological source such as a cell, a tissue, or a biological fluid. In some embodiments, the nHC-HA/PTX3 is purified from a fetal support tissue. In some embodiments the nHC-HA/PTX3 is purified from amniotic membrane. In some embodiments the nHC-HA/PTX3 is purified from umbilical cord. Such complexes are generally assembled in vivo in a subject or ex vivo in cells, tissues, or biological fluids from a subject, including a human or other animal.

As used herein, a PTX3/HA complex refers to an intermediate complex that is formed by contacting PTX3 with immobilized HA. In the methods provided herein, the PTX3/HA complex is generated prior to the addition of HC1 to HA.

As used herein, “hyaluronan,” “hyaluronic acid,” or “hyaluronate” (HA) are used interchangeably to refer to a substantially non-sulfated linear glycosaminoglycan (GAG) with repeating disaccharide units of D-glucuronic acid and N-acetylglucosamine (D-glucuronosyl-N-acetylglucosamine).

As used herein, the term “tissue having unwanted changes” refers to tissue that is degenerated due to, for example, a degenerative disease (for example, arthritis, multiple sclerosis, Parkinson's disease, muscular dystrophy, and Huntington's disease) or aging; scar tissue; damaged due to an insult, such as a burn, wound, laceration, injury, ulcer, radiation, chemotherapy, surgery, or due to ischemia; or diseased (for example a tissue having reduced, impaired or eliminated function due to a disease state such as cancer). In some embodiments, a degenerated tissue has reduced, impaired, or eliminated functional ability relative to a non-degenerated tissue. In some embodiments, a degenerated tissue shows differentiation of a portion of cells of the tissue from a first cell type to a second cell type. An example of a degenerated tissue is cardiac tissue following a myocardial infarction, wherein a portion of the fibroblasts of the cardiac tissue have differentiated into myofibroblasts.

As used herein, the term “mesenchymal cell characteristic of the tissue” refers to specialized cells characteristic of the tissue and differentiated from mesenchymal stem cells, such as, for example, cardiomyocytes, osteoblasts (bone cells), chondrocytes (cartilage cells), myocytes (muscle cells), and adipocytes (fat cells).

As used herein, the term “high molecular weight” or “HMW,” as in high molecular weight hyaluronan (HMW HA), is meant to refer to HA that has a weight average molecular weight that is greater than about 100 kilodaltons (kDa), such as, for example, between about 100 kDa and about 10,000 kDa, between about 500 kDa and about 10,000 kDa, between about 800 kDa and about 8,500 kDa, between about 1100 kDa and about 5,000 kDa, or between about 1400 kDa and about 3,500 kDa. In some embodiments, the BMW HA has a weight average molecular weight of 3000 kDa or greater. In some embodiments, the HMW HA has a weight average molecular weight of 3000 kDa. In some embodiments, the HMW HA is Healon® with a weight average molecular weight of about 3000 kDa. In some embodiments, HMW HA has a molecular weight of between about 100 kDa and about 10,000 kDa. In some embodiments, HMW HA has a molecular weight of between about 500 kDa and about 10,000 kDa. In some embodiments, HMW HA has a molecular weight of between about 800 kDa and about 8,500 kDa. In some embodiments, BMW HA has a molecular weight of about 3,000 kDa.

As used herein, the term “low molecular weight” or “LMW,” as in low molecular weight hyaluronan (LMW HA), is meant to refer to HA that has a weight average molecular weight that is less than 500 kDa, such as for example, less than about 400 kDa, less than about 300 kDa, less than about 200 kDa, less than about 100 kDa, about 100-300 kDa, about 200-300 kDa, or about 1-300 kDa.

As used herein, pentraxin 3, or PTX3, protein or polypeptide refers to any PTX3 protein, including but not limited to, a recombinantly produced protein, a synthetically produced protein, a native PTX3 protein, and a PTX3 protein extracted from cells or tissues. PTX3 includes multimeric forms (e.g. homomultimer) of PTX3, including, but not limited to, dimeric, trimeric, tetrameric, pentameric, hexameric, tetrameric, octameric, and other multimeric forms naturally or artificially produced.

As used herein, tumor necrosis factor stimulated gene-6 (TSG-6) refers to any TSG-6 protein or polypeptide, including but not limited to, a recombinantly produced protein, a synthetically produced protein, a native TSG-6 protein, and a TSG-6 protein extracted from cells or tissues.

As used herein, inter-α-inhibitor (IαI) refers to a IαI protein comprised of light chain (i.e., bikunin) and one or both heavy chains of type HC1 or HC2 covalently connected by a chondroitin sulfate chain. In some embodiments, the source of IαI is from serum or from cells producing IαI e.g., hepatic cells or amniotic epithelial or stromal cells or umbilical epithelial or stromal cells under constitutive mode stimulation by proinflammatory cytokines such as IL-1 or TNF-α.

As used herein, a “hyaluronan binding protein,” “HA binding protein,” or “HABP” refers to any protein that specifically binds to HA.

The terms “effective amount” or “therapeutically effective amount,” as used herein, refer to a sufficient amount of an agent or a compound being administered which will relieve to some extent one or more of the symptoms of the disease or condition being treated. In some embodiments, the result is reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein, is an amount effective to achieve a desired effect or therapeutic improvement without undue adverse side effects. It is understood that “an effective amount” or “a therapeutically effective amount” can vary from subject to subject, due to variation in metabolism of the composition, age, weight, general condition of the subject, the condition being treated, the severity of the condition being treated, and the judgment of the prescribing physician.

As used herein, the terms “subject,” “individual,” and “patient” are used interchangeably. None of the terms are to be interpreted as requiring the supervision of a medical professional (e.g., a doctor, nurse, physician's assistant, orderly, hospice worker). As used herein, the subject is any animal, including mammals (e.g., a human or non-human animal) and non-mammals. In one embodiment of the methods and compositions provided herein, the mammal is a human.

As used herein, the terms “treat,” “treating,” or “treatment,” and other grammatical equivalents, including, but not limited to, alleviating, abating, or ameliorating one or more symptoms of a disease or condition, ameliorating, preventing or reducing the appearance, severity, or frequency of one or more additional symptoms of a disease or condition, ameliorating or preventing the underlying metabolic causes of one or more symptoms of a disease or condition, inhibiting the disease or condition, such as, for example, arresting the development of the disease or condition, relieving the disease or condition, causing regression of the disease or condition, relieving a condition caused by the disease or condition, or inhibiting the symptoms of the disease or condition either prophylactically and/or therapeutically. In a non-limiting example, for prophylactic benefit, an rcHC-HA/PTX3 complex or composition disclosed herein is administered to an individual at risk of developing a particular disorder, predisposed to developing a particular disorder, or to an individual reporting one or more of the physiological symptoms of a disorder.

### Fetal Support Tissue Products

As used herein, the term “preparation” or “product” refers to ground, pulverized, morselized, a graft, a sheet, a powder, a gel, a homogenate, an extract, a terminally-sterilized product derived from a fetal support tissue, purified native HC-HA/PTX3 complex, reconstituted HC-HA/PTX3, or a combination thereof. In some embodiments, the preparation is a fetal support tissue product or an extract of a fetal support tissue. In some embodiments, the fetal support tissue is a placental amniotic membrane, umbilical cord, umbilical cord amniotic membrane, chorion, amnion-chorion, placenta, amniotic stroma, amniotic jelly, or any combination thereof.

In some embodiments, the preparation is an umbilical cord product, an amniotic membrane product, or umbilical cord amniotic membrane product. In some embodiments, the umbilical cord product comprises umbilical cord amniotic membrane and at least some Wharton's jelly. In some embodiments, the umbilical cord product lacks umbilical cord vein and arteries.

In some embodiments, the preparation is an extract of a fetal support tissue. In some embodiments, the preparation is purified native HC-HA/PTX3 complex (nHC-HA/PTX3) from a fetal support tissue. In some embodiments, the preparation is a reconstituted HC-HA/PTX3 complex (rcHC-HA/PTX3). In some embodiments, the preparation consists essentially of nHC-HA/PTX3. In some embodiments, the preparation consists essentially of rcHC-HA/PTX3. In some embodiments, the preparation comprises a combination of nHC-HA/PTX3 and rcHC-HA/PTX3.

In some embodiments, the fetal support tissue product is a UC product. In some embodiments, the fetal support tissue product is an AM product. In some embodiments, the fetal support tissue product is a UCAM product. In some embodiments, the fetal support tissue products comprise: isolated fetal support tissue that does not comprise a vein or an artery. In some embodiments, the fetal support tissue products comprise: isolated fetal support tissue that does not comprise a vein or an artery, a cell with metabolic activity, active HIV-1, active HIV-2, active HTLV-1, active hepatitis B, active hepatitis C, active West Nile Virus, active cytomegalovirus, active human transmissible spongiform encephalopathy, or active Treponema pallidum, wherein the natural structural integrity of the fetal support tissue product is substantially preserved for at least 15 days after initial procurement. In some embodiments, the fetal support tissue product comprises umbilical cord amniotic membrane and Wharton's Jelly. In some embodiments, the biological activity of HC-HA/PTX3 complex in the fetal support tissue product is substantially preserved. In some embodiments, the biological activity of HC-HA/PTX3 complex in the fetal support tissue product is substantially preserved for at least 15 days. In some embodiments, the biological and structural integrity of the fetal support tissue product is substantially preserved for at least 20 days after initial procurement. In some embodiments, the biological and structural integrity of the fetal support tissue product is substantially preserved for at least 25 days after initial procurement. In some embodiments, the biological and structural integrity of the fetal support tissue product is substantially preserved for at least 30 days after initial procurement. In some embodiments, the biological and structural integrity of the fetal support tissue product is substantially preserved for at least 35 days after initial procurement. In some embodiments, the biological and structural integrity of the fetal support tissue product is substantially preserved for at least 40 days after initial procurement. In some embodiments, the biological and structural integrity of the fetal support tissue product is substantially preserved for at least 45 days after initial procurement. In some embodiments, the biological and structural integrity of the fetal support tissue product is substantially preserved for at least 50 days after initial procurement. In some embodiments, the biological and structural integrity of the fetal support tissue product is substantially preserved for at least 55 days after initial procurement. In some embodiments, the biological and structural integrity of the fetal support tissue product is substantially preserved for at least 60 days after initial procurement. In some embodiments, the biological and structural integrity of the fetal support tissue product is substantially preserved for at least 90 days after initial procurement. In some embodiments, the biological and structural integrity of the fetal support tissue product is substantially preserved for at least 180 days after initial procurement. In some embodiments, the biological and structural integrity of the fetal support tissue product is substantially preserved for at least 1 year after initial procurement. In some embodiments, the biological and structural integrity of the fetal support tissue product is substantially preserved for at least 2 years after initial procurement. In some embodiments, the biological and structural integrity of the fetal support tissue product is substantially preserved for at least 3 years after initial procurement. In some embodiments, the biological and structural integrity of the fetal support tissue product is substantially preserved for at least 4 years after initial procurement. In some embodiments, the biological and structural integrity of the fetal support tissue product is substantially preserved for at least 5 years after initial procurement.

Further disclosed herein, in certain embodiments, a method of producing a fetal support tissue product, comprising: obtaining pre-frozen fetal support tissue, wherein the structural integrity of the fetal support tissue product is substantially preserved for at least 15 days after processing. In some embodiments, substantially all of the blood is removed from the fetal support tissue product. In some embodiments, the fetal support tissue is processed by thawing pre-frozen fetal support tissue, and removing substantially all of the blood from the umbilical cord. In some embodiments, the umbilical vein and umbilical arteries are removed from the fetal support tissue. In some embodiments, the biological and structural integrity of the fetal support tissue product is substantially preserved for at least 20 days after processing. In some embodiments, the biological and structural integrity of the fetal support tissue product is substantially preserved for at least 25 days after processing. In some embodiments, the biological and structural integrity of the fetal support tissue product is substantially preserved for at least 30 days after processing. In some embodiments, the biological and structural integrity of the fetal support tissue product is substantially preserved for at least 35 days after processing. In some embodiments, the biological and structural integrity of the fetal support tissue product is substantially preserved for at least 40 days after processing. In some embodiments, the biological and structural integrity of the fetal support tissue product is substantially preserved for at least 45 days after processing. In some embodiments, the biological and structural integrity of the fetal support tissue product is substantially preserved for at least 50 days after processing. In some embodiments, the biological and structural integrity of the fetal support tissue product is substantially preserved for at least 55 days after processing. In some embodiments, the biological and structural integrity of the fetal support tissue product is substantially preserved for at least 60 days after processing. In some embodiments, the biological and structural integrity of the fetal support tissue product is substantially preserved for at least 90 days after processing. In some embodiments, the biological and structural integrity of the fetal support tissue product is substantially preserved for at least 180 days after processing. In some embodiments, the biological and structural integrity of the fetal support tissue product is substantially preserved for at least 1 year after processing. In some embodiments, the biological and structural integrity of the fetal support tissue product is substantially preserved for at least 2 years after processing. In some embodiments, the biological and structural integrity of the fetal support tissue product is substantially preserved for at least 3 years after processing. In some embodiments, the biological and structural integrity of the fetal support tissue product is substantially preserved for at least 4 years after processing. In some embodiments, the biological and structural integrity of the fetal support tissue product is substantially preserved for at least 5 years after processing. In some embodiments, at least a portion of the Wharton's Jelly is removed. In some embodiments, fetal support tissue is recovered from any suitable source (e.g., a hospital or tissue bank). In some embodiments, fetal support tissue is obtained from a mammal. In some embodiments, fetal support tissue is obtained from a human, a non-human primate, a cow or a pig.

In some embodiments, the fetal support tissue product is frozen. In some embodiments, the fetal support tissue product is kept at or below 0° C. until donor and specimen eligibility has been determined. In some embodiments, the fetal support tissue product is kept at or below 0° C., −10° C., −20° C., −30° C., −40° C., −50° C., −60° C., −70° C., or −80° C. In some embodiments, storing the fetal support tissue product at or below 0° C. kills substantially all cells found in the fetal support tissue. In some embodiments, storing the fetal support tissue product at or below 0° C. kills substantially all cells found in the fetal support tissue product while maintaining or increasing the biological activity of the fetal support tissue product (e.g., its anti-inflammatory, anti-scarring, anti-antigenic, and anti-adhesion properties) relative to fresh (i.e., non-frozen) fetal support tissue. In some embodiments, storing the fetal support tissue product at or below 0° C. results in the loss of metabolic activity in substantially all cells found in the fetal support tissue. In some embodiments, the fetal support tissue is dried. In some embodiments, the fetal support tissue is not dehydrated.

### Processing of Fetal Support Tissue

All processing is done following Good Tissue Practices (GTP) to ensure that no contaminants are introduced into the fetal support tissue product.

The fetal support tissue is tested for HIV-1, HIV-2, HTLV-1, hepatitis B and C, West Nile virus, cytomegalovirus, human transmissible spongiform encephalopathy (e.g., Creutzfeldt-Jakob disease), and Treponema pallidum using FDA licensed screening test. Any indication that the tissue is contaminated with HIV-1, HIV-2, HTLV-1, hepatitis B and C, West Nile virus, or cytomegalovirus results in the immediate quarantine and subsequent destruction of the tissue specimen.

Further, the donor's medical records are examined for risk factors for and clinical evidence of hepatitis B, hepatitis C, or HIV infection. Any indication that the donor has risk factors for, and/or clinical evidence of, infection with HIV-1, HIV-2, HTLV-1, hepatitis B and C, West Nile virus, cytomegalovirus, human transmissible spongiform encephalopathy (e.g., Creutzfeldt-Jakob disease), and Treponema pallidum results in the immediate quarantine and subsequent destruction of the tissue specimen.

In some embodiments, the fetal support tissue is frozen. In some embodiments, the fetal support tissue is not frozen. If the fetal support tissue is not frozen, it is processed as described below immediately.

In some embodiments, substantially all of the blood is removed from the fetal support tissue (e.g., from any arteries and veins found in the fetal support tissue, and blood that has infiltrated into the tissue). In some embodiments, substantially all of the blood is removed before the fetal support tissue is frozen. In some embodiments, blood is not removed from the fetal support tissue. In some embodiments, blood is not removed from the fetal support tissue before the fetal support tissue is frozen. In some embodiments, the blood is substantially removed after the fetal support tissue has been frozen.

In some embodiments, the fetal support tissue is washed with buffer with agitation to remove excess blood and tissue. In some embodiments, the fetal support tissue is soaked with buffer with agitation to remove excess blood and tissue. In some embodiments, washing or soaking with agitation reduces the wash time. In some embodiments, the buffer wash solution is exchanged for fresh buffer solution. In some embodiments, the buffer is optionally changed during the contacting (e.g., when the rate at which red blood cells diffuse from the fetal support tissue slows). In some embodiments, a magnetic stirrer is added during the contacting. In some embodiments, adding (and activating) a magnetic stirrer increases the rate at which the red blood cells diffuse from the fetal support tissue. In some embodiments, the fetal support tissue is soaked in isotonic solution and the solution is exchanged. In some embodiments, the fetal support tissue is washed with an isotonic buffer or tissue culture media. In some embodiments, the fetal support tissue is washed with saline. In some embodiments, the fetal support tissue is washed with PBS. In some embodiments, the fetal support tissue is washed with 1×PBS. In some embodiments, the fetal support tissue is washed with a TRIS-buffered saline. In some embodiments, the fetal support tissue is washed with a HEPES-buffered saline. In some embodiments, the fetal support tissue is washed with Ringer's solution. In some embodiments, the fetal support tissue is washed with Ringer's lactate solution. In some embodiments, the fetal support tissue is washed with Hartmann's solution. In some embodiments, the fetal support tissue is washed with EBSS. In some embodiments, the fetal support tissue is washed with HBSS. In some embodiments, the fetal support tissue is washed with Tyrode's Salt Solution. In some embodiments, the fetal support tissue is washed with Gey's Balanced Salt Solution. In some embodiments, the fetal support tissue is washed with DMEM. In some embodiments, the fetal support tissue is washed with EMEM. In some embodiments, the UC is washed with GMEM. In some embodiments, the fetal support tissue is washed with RPMI.

In some embodiments, the use is a homologous use (e.g., a functional homologous use or a structural homologous use). In some embodiments, the fetal support tissue product is minimally manipulated. In some embodiments, the fetal support tissue product does not comprise another article, except for water, crystalloids, or a sterilizing, preserving, or storage agent. In some embodiments, the fetal support tissue product does not have a systemic effect and is not dependent upon the metabolic activity of living cells for its primary function.

### Processing to Generate a Fetal Support Tissue Graft

In some embodiments, the fetal support tissue product is a fetal support tissue graft. In some embodiments, isolated fetal support tissue is used to generate a fetal support tissue graft. In some embodiments, the fetal support tissue is cut into multiple sections (e.g., using a scalpel). The size of the sections depends on the desired use of the fetal support tissue graft derived from the fetal support tissue. In some embodiments, the cut fetal support tissue is optionally washed again with buffer to further remove excess blood and tissue.

In some embodiments, the fetal support tissue graft is derived from an umbilical cord (UC) tissue. In some embodiments, the section of the umbilical cord is cut longitudinally (e.g., using a scalpel or scissors) to open the UC. In some embodiments, the section of the UC is not cut into halves. In some embodiments, the section of the UC is cut into two halves. In some embodiments, additional cuts are made in the Wharton's Jelly to help flatten out the UC. In some embodiments, UC is fastened onto a substrate (e.g., a Styrofoam board) using any suitable method (e.g., it is fastened with needles or pins (e.g., T pins)). In some embodiments, both ends of the umbilical cord are fastened to the substrate. In some embodiments, only one end is attached to the substrate. In some embodiments, the UC is stabilized with a substrate (e.g., absorbent towel cloth, drape). In some embodiments, the UC is oriented such that the inside face of the UC (e.g., the face comprising the Wharton's Jelly) is facing up while the outside face (i.e., the face comprising UCAM) is facing the substrate. If one end of the umbilical cord is left free, in some embodiments, the free end of the umbilical cord is held (e.g., with a clamp, hemostats or a set of forceps (e.g., wide serrated tip forceps)) while part or all of the Wharton's Jelly is removed. Alternatively, in some embodiments, both ends of the UC are left free.

The umbilical cord comprises two arteries (the umbilical arteries) and one vein (the umbilical vein). In some embodiments, the vein and arteries are removed from the UC. In certain instances, the vein and arteries are surrounded (or suspended or buried) within the Wharton's Jelly. In some embodiments, the vein and arteries are removed concurrently with the removal of the Wharton's Jelly. In some embodiments, the vein and arteries are peeled (or pulled) from the umbilical cord (e.g., using a set of forceps). In some embodiments, the vein and arteries are cut away (e.g., shaved) from the umbilical cord in sections. In some embodiments, a rotoblator removes the vein and arteries concurrently with the Wharton's Jelly. In some embodiments, a liposuction machine is utilized to remove the vein and arteries concurrently with the Wharton's Jelly. In some embodiments, a vein stripper is utilized to remove the vein and arteries concurrently with the Wharton's Jelly. In some embodiments, a liquid under high pressure removes the vein and arteries concurrently with the Wharton's Jelly. In some embodiments, a brush removes the vein and arteries concurrently with the Wharton's Jelly. In some embodiments, a surgical dermatome removes the vein and arteries concurrently with the Wharton's Jelly.

In some embodiments, the UC product comprises UCAM as a scaffold, and a plurality of cells integrated into the scaffold. In some embodiments, the cells are embryonic stem cells, mesenchymal stem cells, or adult lineage-committed stem cells, or differentiated epidermal cells (e.g., to treat a burn or a surgical incision in the skin). In some embodiments, the cells are mesothelial cells (e.g., to treat to a wound (e.g., surgical incision) in an internal organ).

In some embodiments, the fetal support tissue graft is derived from an amniotic membrane tissue. In some embodiments, the amniotic membrane tissue is obtained from a placenta. In some embodiments, the placenta has had the chorion removed. In some embodiments, the amniotic membrane graft is used as a scaffold, and a plurality of cells integrated into the scaffold. In some embodiments, the cells are embryonic stem cells, mesenchymal stem cells, or adult lineage-committed stem cells, or differentiated epidermal cells (e.g., to treat a burn or a surgical incision in the skin). In some embodiments, the cells are mesothelial cells (e.g., to treat to a wound (e.g., surgical incision) in an internal organ).

In some embodiments, the fetal support tissue products are in any suitable shape (e.g., a square, a circle, a triangle, a rectangle). In some embodiments, the fetal support tissue product is generated from a sheet of fetal support tissue. In some embodiments, the sheet is flat. In some embodiments, the sheet is tubular.

The size of the fetal support tissue graft depends on the desired use of the fetal support tissue graft. In some embodiments, the fetal support tissue product is cut into multiple sections (e.g., using a scalpel). In some embodiments, the fetal support tissue product is divided into sections that are about 1.0 cm×about 0.25 cm. In some embodiments, the fetal support tissue product is divided into sections that are about 1.0 cm×about 0.5 cm. In some embodiments, the fetal support tissue product is divided into sections that are about 1.0 cm×about 0.75 cm. In some embodiments, the fetal support tissue product is divided into sections that are about 1 cm×about 1 cm. In some embodiments, the fetal support tissue product is divided into sections that are about 1 cm×about 2 cm. In some embodiments, the fetal support tissue product is divided into sections that are about 1 cm×about 3 cm. In some embodiments, the fetal support tissue product is divided into sections that are about 1 cm×about 4 cm. In some embodiments, the fetal support tissue product is divided into sections that are about 1 cm×about 5 cm. In some embodiments, the fetal support tissue product is divided into sections that are about 1 cm×about 6 cm. In some embodiments, the fetal support tissue product is divided into sections that are about 2 cm×about 2 cm. In some embodiments, the fetal support tissue product is divided into sections that are about 2 cm×about 3 cm. In some embodiments, the fetal support tissue product is divided into sections that are about 2 cm×about 4 cm. In some embodiments, the fetal support tissue product is divided into sections that are about 2 cm×about 5 cm. In some embodiments, the fetal support tissue product is divided into sections that are about 2 cm×about 6 cm. In some embodiments, the fetal support tissue product is divided into sections that are about 3 cm×about 3 cm. In some embodiments, the fetal support tissue product is divided into sections that are about 3 cm×about 4 cm. In some embodiments, the fetal support tissue product is divided into sections that are about 3 cm×about 5 cm. In some embodiments, the fetal support tissue product is divided into sections that are about 3 cm×about 6 cm. In some embodiments, the fetal support tissue product is divided into sections that are about 4 cm×about 4 cm. In some embodiments, the fetal support tissue product is divided into sections that are about 4 cm×about 5 cm. In some embodiments, the fetal support tissue product is divided into sections that are about 4 cm×about 6 cm. In some embodiments, the fetal support tissue product is divided into sections that are about 5 cm×about 5 cm. In some embodiments, the fetal support tissue product is divided into sections that are about 5 cm×about 6 cm. In some embodiments, the fetal support tissue product is divided into sections that are about 6 cm×about 6 cm. In some embodiments, the fetal support tissue product is divided into sections that are about 8 cm×about 1 cm. In some embodiments, the fetal support tissue product is divided into sections that are about 8 cm×about 2 cm. In some embodiments, the fetal support tissue product is divided into sections that are about 8 cm×about 3 cm. In some embodiments, the fetal support tissue product is divided into sections that are about 8 cm×about 4 cm. In some embodiments, the fetal support tissue product is divided into sections that are about 8 cm×about 5 cm. In some embodiments, the fetal support tissue product is divided into sections that are about 8 cm×about 6 cm. In some embodiments, the fetal support tissue product is divided into sections that are about 10 cm×about 10 cm. In some embodiments, the fetal support tissue product is divided into sections that are about 12 cm×about 10 cm. In some embodiments, the fetal support tissue product is divided into sections that are about 15 cm×about 10 cm. In some embodiments, the fetal support tissue product is divided into sections that are about 20 cm×about 10 cm. In some embodiments, the fetal support tissue product is divided into sections that are about 25 cm×about 10 cm. In some embodiments, the fetal support tissue product is divided into sections that are about 30 cm×about 10 cm.

### Processing to Generate Morselized Fetal Support Tissue Product

In some embodiments, isolated fetal support tissue is used to generate a morselized fetal support tissue product. As used herein, “morsel” refers to particles of tissue ranging in size from about 0.1 mm to about 1.0 cm in length, width, or thickness that have been obtained from a larger piece of tissue. A “morsel” as described herein, retains the characteristics of the tissue from which it was obtained and upon inspection is identifiable as said tissue. As used herein, the terms “morselized,” “morselizing,” and “morselization” refer to actions involving the “morsels” of the present application. In some embodiments, the morselized fetal support tissue product is further processed into a solution, suspension, or emulsion by mixing the morselized fetal support tissue with a carrier. In some embodiments, the morselized fetal support tissue product is formulated into a cream, lotion, ointment, paste, gel, film, or paint. In some embodiments, the morselized fetal support tissue product is contacted with a patch or wound dressing.

In some embodiments, a mixture of amniotic membrane tissue and umbilical cord tissue in any ratio from 0.001:99.999 w/w % to 99.999:0.001 w/w % is morselized from either fresh or frozen tissue through the use of any morselizing tool known to one of skill in the art such as, for example, tissue grinder, sonicator, bread beater, freezer/mill, blender, mortar/pestle, Roto-stator, kitchen chopper, grater, ruler, and scalpel to yield morsels ranging in size from about 0.1 mm to about 1.0 cm in length, width, or thickness. In some embodiments, the resulting morsels are homogenized to yield consistently sized morsels. In some embodiments, the resulting morsels are used wet, partially dehydrated, or essentially dehydrated by any means known to one of skill in the art such as, for example, centrifuge or lyophilization. In some embodiments, the resulting preparation is used immediately or stored for later use in any type of container known to one of skill in the art such as, for example, pouch, jar, bottle, tube, ampule, and pre-filled syringe. In some embodiments, the morselized preparation is sterilized by any method known to one of skill in the art such as, for example, γ radiation.

In some embodiments, the isolated fetal support tissue is optionally lyophilized before being morselized. In some embodiments, the isolated fetal support tissue is lyophilized by any suitable method (e.g., exposure to a liquid gas, placement in a freezer). In some embodiments, the isolated fetal support tissue is placed in the vacuum chamber of a lyophilization device until all or substantially all fluid (e.g., water) has been removed. In some embodiments, the isolated fetal support tissue is lyophilized following freezing (e.g., exposure to a temperature below 0° C., −20° C., −40° C., −50° C., −60° C., −70° C., −75° C., −80° C., −90° C., or −100° C.).

### Processing to Generate Pulverized Fetal Support Tissue Product

In some embodiments, isolated fetal support tissue is used to generate a pulverized fetal support tissue product. As used herein, “pulverized fetal support tissue product” means a fetal support tissue product comprising tissue that has been broken up (or, disassociated). In some embodiments, the pulverized fetal support tissue product is a dry powder. In some embodiments, the pulverized fetal support tissue product is further processed into a solution, suspension, or emulsion by mixing the fetal support tissue powder with a carrier. In some embodiments, the pulverized fetal support tissue product is formulated into a cream, lotion, ointment, paste, gel, film or paint. In some embodiments, the pulverized fetal support tissue product is contacted with a patch or wound dressing.

In some embodiments, the isolated fetal support tissue is pulverized by any suitable method. In some embodiments, the isolated fetal support tissue is pulverized by use of a pulverizer (e.g., a Bessman Tissue Pulverizer, a Biospec biopulverizer, or a Covaris CryoPrep). In some embodiments, the isolated fetal support tissue is pulverized by use of a tissue grinder (e.g., a Potter-Elvehjem grinder or a Wheaton Overhead Stirrer). In some embodiments, the isolated fetal support tissue is pulverized by use of a sonicator. In some embodiments, the isolated fetal support tissue is pulverized by use of a bead beater. In some embodiments, the isolated fetal support tissue is pulverized by use of a freezer/mill (e.g., a SPEX SamplePrep Freezer/Mill or a Retch Ball Mill). In some embodiments, the isolated fetal support tissue is pulverized by use of a pestle and mortar. In some embodiments, the isolated fetal support tissue is pulverized by manual use of a pestle and mortar.

In some embodiments, the isolated fetal support tissue is optionally lyophilized before being pulverized. In some embodiments, the isolated fetal support tissue is lyophilized by any suitable method (e.g., exposure to a liquid gas, placement in a freezer). In some embodiments, the isolated fetal support tissue is placed in the vacuum chamber of a lyophilization device until all or substantially all fluid (e.g., water) has been removed. In some embodiments, the isolated fetal support tissue is lyophilized following freezing (e.g., exposure to a temperature below 0° C., −20° C., −40° C., −50° C., −60° C., −70° C., −75° C., −80° C., −90° C., or −100° C.).

### Storage of the Fetal Support Tissue Product

In some embodiments, the fetal support tissue product is stored for later use. In some embodiments, storing the fetal support tissue product does not destroy the integrity of the fetal support tissue extracellular matrix. In some embodiments, the fetal support tissue product is lyophilized. In some embodiments, the fetal support tissue product is stored in any suitable storage medium. In some embodiments, the fetal support tissue product is stored in 50% DMEM+50% Glycerol. In some embodiments, the fetal support tissue product is stored in 5%, 10%, 20%, 30%, 40%, 50%, 60%, 70%, 80%, 90%, or 100% glycerol. In some embodiments, the fetal support tissue product is stored in 5%, 10%, 20%, 30%, 40%, 50%, 60%, 70%, 80%, 90%, or 100% propylene glycol. In some embodiments, the % glycerol or % propylene glycol is the percent weight per volume (w/v) or percent volume per volume (v/v) of glycerol or propylene glycol, respectively, in a solution. In some embodiments, the fetal support tissue product is stored in saline solution.

In some embodiments, the fetal support tissue product is optionally contacted with a substrate (i.e., a supportive backing). In some embodiments, the fetal support tissue product is not contacted with a substrate. In some embodiments, the fetal support tissue product is orientated such that the fetal support tissue product is in contact with the substrate. In some embodiments, the fetal support tissue product is orientated such that the stroma is in contact with the substrate. In some embodiments the fetal support tissue product is orientated such that the epithelial side is in contact with the substrate.

In some embodiments, the fetal support tissue product is attached to the substrate. In some embodiments, the substrate is nitrocellulose paper (NC). In some embodiments, the substrate is nylon membrane (NM). In some embodiments, the substrate is polyethersulfone membrane (PES).

### Cryopreservation

In some embodiments, the fetal support tissue product is frozen for cryopreservation. In some embodiments, cryopreserving the fetal support tissue product does not destroy the integrity of the fetal support tissue extracellular matrix. In some embodiments, the fetal support tissue product is exposed to a liquid gas (e.g., liquid nitrogen or liquid hydrogen). In some embodiments, the fetal support tissue product is exposed to liquid nitrogen. In some embodiments, the fetal support tissue product does not contact the liquid gas. In some embodiments, the fetal support tissue product is placed in a container and the container is contacted with liquid gas. In some embodiments, the fetal support tissue product is exposed to the liquid gas until the fetal support tissue product is frozen.

### Lyophilization

In some embodiments, the fetal support tissue product is lyophilized. In some embodiments, the fetal support tissue product is lyophilized following freezing. In some embodiments, the fetal support tissue product is lyophilized following freezing by any suitable method (e.g., exposure to a liquid gas, placement in a freezer). In some embodiments, the fetal support tissue product is frozen by exposure to a temperature below about 0° C. In some embodiments, the fetal support tissue product is frozen by exposure to a temperature below about −20° C. In some embodiments, the fetal support tissue product is frozen by exposure to a temperature below about −40° C. In some embodiments, the fetal support tissue product is frozen by exposure to a temperature below about −50° C. In some embodiments, the fetal support tissue product is frozen by exposure to a temperature below about −60° C. In some embodiments, the fetal support tissue product is frozen by exposure to a temperature below about −70° C. In some embodiments, the fetal support tissue product is frozen by exposure to a temperature below about −75° C. In some embodiments, the fetal support tissue product is frozen by exposure to a temperature below about −80° C. In some embodiments, the fetal support tissue product is frozen by exposure to a temperature below about −90° C. In some embodiments, the fetal support tissue product is frozen by exposure to a temperature below about −100° C. In some embodiments, the fetal support tissue product is frozen by exposure to a liquid gas.

In some embodiments, the cryopreserved fetal support tissue product is lyophilized. In some embodiments, the cryopreserved fetal support tissue product is placed in the vacuum chamber of a lyophilization device until all or substantially all fluid (e.g., water) has been removed.

### Grinding

In some embodiments, the lyophilized fetal support tissue is ground by any suitable method. Duration and frequency of grinding may be varied according to the desired outcome. It is within the skills of one skilled in the art to determine the necessary parameters. As used herein, “grinding” means any method of reducing fetal support tissue to small particles or a powder. The term grinding includes micronizing, pulverizing, homogenizing, filing, milling, grating, pounding, and crushing.

In some embodiments, the lyophilized fetal support tissue is ground by use of a grinding container. In some embodiments, the lyophilized fetal support tissue is ground by use of a pulverizer (e.g., a Bessman Tissue Pulverizer or a Covaris CryoPrep). In some embodiments, the lyophilized fetal support tissue is ground by use of a tissue grinder (e.g., a Potter-Elvehjem grinder or a Wheaton Overhead Stirrer). In some embodiments, the lyophilized fetal support tissue is ground by use of a sonicator. In some embodiments, the lyophilized fetal support tissue is ground by use of a bead beater. In some embodiments, the lyophilized fetal support tissue is ground by use of a freezer/mill (e.g., a SPEX SamplePrep Freezer/Mill). In some embodiments, lyophilized fetal support tissue is ground by use of a pestle and mortar. In some embodiments, the lyophilized fetal support tissue is ground by manual use of a pestle and mortar.

In some embodiments, the lyophilized fetal support tissue is ground by use of a grinding container. In some embodiments, the fetal support tissue is ground at a frequency of between about 10 Hz and about 25 Hz. In some embodiments, the fetal support tissue is ground at a frequency of about 10 Hz. In some embodiments, the fetal support tissue is ground at a frequency of about 15 Hz. In some embodiments, the fetal support tissue is ground at a frequency of about 20 Hz. In some embodiments, the fetal support tissue is ground at a frequency of about 25 Hz. In some embodiments, grinding lasts for any suitable time period. The lower the grinding frequency, the greater the amount of time required to grind the lyophilized fetal support tissue. The duration of grinding varies with the desired form of the powder. In some embodiments, grinding lasts for between about 1 and about 6 minutes, for example about 1 minute, about 2 minutes, about 3 minutes, about 4 minutes, about 5 minutes, or about 6 minutes.

In some embodiments, grinding the lyophilized fetal support tissue further comprises continuously freezing the lyophilized fetal support tissue. For example, in some embodiments, the lyophilized fetal support tissue is placed in a grinding container and the grinding container is exposed to temperatures below 0° C. (e.g., the grinding container is immersed in liquid nitrogen or the container comprises an automated liquid nitrogen cooling feature).

In some embodiments, the grinding the lyophilized fetal support tissue produces a powder. As used herein, “powder” means matter in the form of fine dry particles or matrix. In some embodiments, the particles are not uniform in size. In some embodiments, the particles are substantially uniform in size.

In some embodiments, the fetal support tissue is divided into pieces prior to lyophilization. In some embodiments, the lyophilized fetal support tissue is divided into pieces prior to grinding. In some embodiments, the powder is frozen. In some embodiments, the powder is stored at ambient temperature. In some embodiments, the powder is aliquoted. In some embodiments, the powder is a) frozen; b) thawed; and c) aliquoted. In some embodiments, the powder is aliquoted without prior freezing. In some embodiments, the powder is stored at ambient temperature prior to being aliquoted. In some embodiments, the aliquoted powder is packaged into a packet, a vial, a pre-filled syringe, or a bottle.

### Sterilization

In some embodiments, the fetal support tissue product is subject to terminal sterilization by any suitable (e.g., medically acceptable) method. In some embodiments, the lyophilized fetal support tissue product is exposed to gamma radiation for a period of time sufficient to sterilize the fetal support tissue product. In some embodiments, the lyophilized fetal support tissue product is exposed to gamma radiation at 25 kGy for a period of time sufficient to sterilize the fetal support tissue product. In some embodiments, the lyophilized fetal support tissue product is exposed to an electron beam for a period of time sufficient to sterilize the fetal support tissue product. In some embodiments, the lyophilized fetal support tissue product is exposed to X-ray radiation for a period of time sufficient to sterilize the fetal support tissue product. In some embodiments, the lyophilized fetal support tissue product is exposed to UV radiation for a period of time sufficient to sterilize the fetal support tissue product.

### Rehydration

In some embodiments, the fetal support tissue product is partially or fully rehydrated. In some embodiments, the fetal support tissue product is rehydrated by contacting the fetal support tissue product with a buffer or with water. In some embodiments, the fetal support tissue product is contacted with an isotonic buffer. In some embodiments, the fetal support tissue is contacted with saline. In some embodiments, the fetal support tissue product is contacted with PBS. In some embodiments, the fetal support tissue product is contacted with Ringer's solution. In some embodiments, the fetal support tissue product is contacted with Hartmann's solution. In some embodiments, the fetal support tissue product is contacted with a TRIS-buffered saline. In some embodiments, the fetal support tissue product is contacted with a HEPES-buffered saline; 50% DMEM+50% Glycerol; 10%, 20%, 30%, 40%, 50%, 60%, 70%, 80%, 90%, or 100% glycerol; and/or 10%, 20%, 30%, 40%, 50%, 60%, 70%, 80%, 90%, or 100% propylene glycol.

In some embodiments, the fetal support tissue product is contacted with a buffer for 10 minutes. In some embodiments, the fetal support tissue product is contacted with a buffer for 15 minutes. In some embodiments, the fetal support tissue product is contacted with a buffer for 20 minutes. In some embodiments, the fetal support tissue product is contacted with a buffer for 25 minutes. In some embodiments, the fetal support tissue product is contacted with a buffer for 30 minutes. In some embodiments, the fetal support tissue product is contacted with a buffer for 35 minutes. In some embodiments, the fetal support tissue product is contacted with a buffer for 40 minutes. In some embodiments, the fetal support tissue product is contacted with a buffer for 45 minutes. In some embodiments, the fetal support tissue product is contacted with a buffer for 50 minutes. In some embodiments, the fetal support tissue product is contacted with a buffer for 55 minutes. In some embodiments, the fetal support tissue product is contacted with a buffer for 60 minutes. In some embodiments, the fetal support tissue product is contacted with a buffer for 2 hours. In some embodiments, the fetal support tissue product is contacted with a buffer for 3 hours. In some embodiments, the fetal support tissue product is contacted with a buffer for 4 hours. In some embodiments, the fetal support tissue product is contacted with a buffer for 5 hours. In some embodiments, the fetal support tissue product is contacted with a buffer for 6 hours. In some embodiments, the fetal support tissue product is contacted with a buffer for 6 hours. In some embodiments, the fetal support tissue product is contacted with a buffer for 10 hours. In some embodiments, the fetal support tissue product is contacted with a buffer for 12 hours. In some embodiments, the fetal support tissue product is contacted with a buffer for 18 hours. In some embodiments, the fetal support tissue product is contacted with a buffer for 24 hours.

Methods of Production of Isolated nHC-HA/PTX3 Complexes

In some embodiments, the isolated nHC-HA/PTX3 complex is isolated from an amniotic tissue. In some embodiments, the isolated nHC-HA/PTX3 complex is isolated from an amniotic membrane or an umbilical cord. In some embodiments, the isolated nHC-HA/PTX3 complex is isolated from fresh, frozen, or previously frozen placental amniotic membrane (PAM), fresh, frozen, or previously frozen umbilical cord amniotic membrane (UCAM), fresh, frozen, or previously frozen placenta, fresh, frozen, or previously frozen umbilical cord, fresh, frozen, or previously frozen chorion, fresh, frozen, or previously frozen amnion-chorion, or any combinations thereof. In some embodiments, such tissues are obtained from any mammal, such as, for example, but not limited to a human, non-human primate, cow, or pig.

In some embodiments, the nHC-HA/PTX3 is purified by any suitable method. In some embodiments, the nHC-HA/PTX3 complex is purified by centrifugation (e.g., ultracentrifugation, gradient centrifugation), chromatography (e.g., ion exchange, affinity, size exclusion, and hydroxyapatite chromatography), gel filtration, or differential solubility, ethanol precipitation, or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997, all incorporated herein by reference).

In some embodiments, the nHC-HA/PTX3 is isolated from an extract. In some embodiments, the extract is prepared from an amniotic membrane extract. In some embodiments, the extract is prepared from an umbilical cord extract. In some embodiments, the umbilical cord extract comprises umbilical cord stroma and/or Wharton's jelly. In some embodiments, the nHC-HA/PTX3 complex is contained in an extract that is prepared by ultracentrifugation. In some embodiments, the nHC-HA/PTX3 complex is contained in an extract that is prepared by ultracentrifugation using a CsCl/4-6M guanidine HCl gradient. In some embodiments, the extract is prepared by at least 2 rounds of ultracentrifugation. In some embodiments, the extract is prepared by more than 2 rounds of ultracentrifugation (i.e. nHC-HA/PTX3 2nd). In some embodiments, the extract is prepared by at least 4 rounds of ultracentrifugation (i.e. nHC-HA/PTX3 4th). In some embodiments, the nHC-HA/PTX3 complex comprises a small leucine-rich proteoglycan. In some embodiments, the nHC-HA/PTX3 complex comprises HC1, HA, PTX3, and/or a small leucine-rich proteoglycan.

In some embodiments, ultracentrifugation is performed on an extract prepared by extraction in an isotonic solution. In some embodiments, the isotonic solution is PBS. For example, in some embodiments, the tissue is homogenized in PBS to produce a homogenized sample. In some embodiments, the homogenized sample is then separated into a soluble portion and insoluble portion by centrifugation. In some embodiments, ultracentrifugation is performed on the soluble portion of the PBS-extracted tissue. In such embodiments, the nHC-HA/PTX3, purified by ultracentrifugation of the PBS-extracted tissue is called an nHC-HA/PTX3 soluble complex. In some embodiments, the nHC-HA soluble complex comprises a small leucine-rich proteoglycan. In some embodiments, the nHC-HA/PTX3 soluble complex comprises HC1, HA, PTX3, and/or a small leucine-rich proteoglycan.

In some embodiments, ultracentrifugation is performed on an extract prepared by direct guanidine HCl extraction (e.g. 4-6 M GnHCl) of the amniotic membrane and/or umbilical cord tissue. In some embodiments, the GnHCl extracted tissues are then centrifuged to produce GnHCl soluble and GnHCl insoluble portions. In some embodiments, ultracentrifugation is performed on the GnHCl soluble portion. In such embodiments, the nHC-HA/PTX3 purified by ultracentrifugation of the guanidine HCl-extracted tissue is called an nHC-HA/PTX3 insoluble complex. In some embodiments, the nHC-HA insoluble complex comprises a small leucine-rich proteoglycan. In some embodiments, the nHC-HA/PTX3 insoluble complex comprises HC1, HA, PTX3 and/or a small leucine-rich proteoglycan.

In some embodiments, ultracentrifugation is performed on an extract prepared by further guanidine HCl extraction of the insoluble portion of the PBS-extracted tissue. For example, in some embodiments, the tissue is homogenized in PBS to produce a homogenized sample. In some embodiments, the homogenized sample is then separated into a soluble portion and insoluble portion by centrifugation. In some embodiments, the insoluble portion is then further extracted in guanidine HCl (e.g. 4-6 M GnHCl) and centrifuged to produce guanidine HCl soluble and insoluble portions. In some embodiments, ultracentrifugation is performed on the guanidine HCl soluble portion. In such embodiments, the nHC-HA/PTX3 purified by ultracentrifugation of the guanidine HCl-extracted tissue is called an nHC-HA/PTX3 insoluble complex. In some embodiments, the nHC-HA insoluble complex comprises a small leucine-rich proteoglycan. In some embodiments, the nHC-HA/PTX3 insoluble complex comprises HC1, HA, PTX3, and/or a small leucine-rich proteoglycan.

In some embodiments, the method of purifying the isolated nHC-HA/PTX3 extract comprises: (a) dissolving the isolated extract (e.g. prepared by the soluble or insoluble method described herein) in CsCl/4-6M guanidine HCl at the initial density of 1.35 g/ml, to generate a CsCl mixture; (b) centrifuging the CsCl mixture at 125,000×g for 48 h at 15° C., to generate a first purified extract; (c) extracting the first purified extract and dialyzing it against distilled water to remove CsCl and guanidine HCl, to generate a dialysate. In some embodiments, the method of purifying the isolated extract further comprises: (d) mixing the dialysate with 3 volumes of 95% (v/v) ethanol containing 1.3% (w/v) potassium acetate at 0° C. for 1 h, to generate a first dialysate/ethanol mixture; (e) centrifuging the first dialysate/ethanol mixture at 15,000×g, to generate a second purified extract; and (f) extracting the second purified extract. In some embodiments, the method of purifying the isolated extract further comprises: (g) washing the second purified extract with ethanol (e.g., 70% ethanol), to generate a second purified extract/ethanol mixture; (h) centrifuging the second purified extract/ethanol mixture, to generate a third purified extract; and (i) extracting the third purified extract. In some embodiments, the method of purifying the isolated extract further comprises: (j) washing the third purified extract with ethanol (e.g., 70% ethanol), to generate a third purified extract/ethanol mixture; (k) centrifuging the third purified extract/ethanol mixture, to generate a forth purified extract; and (l) extracting the forth purified extract. In some embodiments, the purified extract comprises an nHC-HA/PTX3 complex.

In some embodiments, the nHC-HA/PTX3 complex is purified by immunoaffinity chromatography. In some embodiments, anti-HC1 antibodies, anti-HC2 antibodies, or both are generated and affixed to a stationary support. In some embodiments, the unpurified HC-HA complex (i.e., the mobile phase) is passed over the support. In certain instances, the HC-HA complex binds to the antibodies (e.g., via interaction of (a) an anti-HC1 antibody and HC1, (b) an anti-HC2 antibody and HC2, (c) an anti-PTX3 antibody and PTX3, (d) an anti-SLRP antibody and the SLRP, or (e) any combination thereof). In some embodiments the support is washed (e.g., with PBS) to remove any unbound or loosely bound molecules. In some embodiments, the support is then washed with a solution that enables elution of the nHC-HA/PTX3 complex from the support (e.g., 1% SDS, 6M guanidine-HCl, or 8M urea).

In some embodiments, the nHC-HA/PTX3 complex is purified by affinity chromatography. In some embodiments, HABP is generated and affixed to a stationary support. In some embodiments, the unpurified nHC-HA/PTX3 complex (i.e., the mobile phase) is passed over the support. In certain instances, the nHC-HA/PTX3 complex binds to the HABP. In some embodiments the support is washed (e.g., with PBS) to remove any unbound or loosely bound molecules. In some embodiments, the support is then washed with a solution that enables elution of the HC-HA complex from the support.

In some embodiments, the nHC-HA/PTX3 complex is purified by a combination of HABP affinity chromatography, and immunoaffinity chromatography using anti-HC1 antibodies, anti-HC2 antibodies, anti-PTX3 antibodies, antibodies against a SLRP or a combination of SLRPs, or any combination of antibodies thereof.

In some embodiments, the nHC-HA/PTX3 complex is purified from the insoluble fraction as described herein using one or more antibodies. In some embodiments, the nHC-HA/PTX3 complex is purified from the insoluble fraction as described herein using anti-SLRP antibodies.

In some embodiments, the nHC-HA/PTX3 complex is purified from the soluble fraction as described herein. In some embodiments, the nHC-HA/PTX3 complex is purified from the soluble fraction as described herein using anti-PTX3 antibodies.

In some embodiments, the nHC-HA/PTX3 complex comprises a small leucine rich proteoglycan (SLRP). In some embodiments, the nHC-HA/PTX3 complex comprises a class I, class II, or class III SLRP. In some embodiments, the small leucine-rich proteoglycan is selected from among class I SLRPs, such as decorin and biglycan. In some embodiments, the small leucine-rich proteoglycan is selected from among class II SLRPs, such as fibromodulin, lumican, PRELP (proline arginine rich end leucine-rich protein), keratocan, and osteoadherin. In some embodiments, the small leucine-rich proteoglycan is selected from among class III SLRPs, such as epipycan and osteoglycin. In some embodiments, the small leucine-rich proteoglycan is selected from among bikunin, decorin, biglycan, and osteoadherin. In some embodiments, the small leucine-rich protein comprises a glycosaminoglycan. In some embodiments, the small leucine-rich proteoglycan comprises keratan sulfate.

Methods of Production of rcHC-HA/PTX3 Complexes

In some embodiments, a method for generating reconstituted HC-HA/PTX3 complexes comprises contacting a PTX3/HA complex with IαI and TSG-6. In some embodiments, TSG-6 catalyzes the transfer of heavy chain 1 (HC1) of inter-α-inhibitor (IαI) to HA. Provided herein are rcHC-HA/PTX3 complexes produced by such method. In some embodiments, HC1 of IαI forms a covalent linkage with HA.

In some embodiments, a method for generating reconstituted HC-HA/PTX3 complexes comprises (a) contacting high molecular weight hyaluronan (HMW HA) with IαI and TSG-6 to HA to form a HC-HA complex pre-bound to TSG-6, and (b) contacting the HC-HA complex with pentraxin 3 (PTX3) under suitable conditions to form a rcHC-HA/PTX3 complex. Provided herein are rcHC-HA/PTX3 complexes produced by such method. In some embodiments, HC1 of IαI forms a covalent linkage with HA. In some embodiments, the steps (a) and (b) of the method are performed sequentially in order. In some embodiments, the method comprises contacting an HC-HA complex pre-bound to TSG-6 with PTX3.

In some embodiments, the method comprises first contacting high molecular weight hyaluronan (HMW HA) with pentraxin 3 (PTX3) under suitable conditions to form a PTX3/HA complex, then contacting the PTX3/HA complex with IαI and TSG-6.

In some embodiments, the IαI protein and TSG-6 protein are contacted to the complex at a molar ratio of about 1:1, 2:1, 3:1, 4:1, 5:1, 6:1, 7:1, 8:1, 9:1, 10:1, 15:1, or 20:1 (IαI:TSG-6). In some embodiments the ratio of IαI:TSG-6 ranges from about 1:1 to about 20:1, such as about 1:1 to about 10:1, such as about 1:1 to 5 about:1, such as about 1:1 to about 3:1. In some embodiments, the ratio of IαI:TSG-6 is 3:1 or higher. In some embodiments, the ratio of IαI:TSG-6 is 3:1.

In some embodiments, the steps (a) and (b) of the method are performed sequentially in order. In some embodiments, the method comprises contacting a PTX3/HA complex with IαI and TSG-6.

In certain instances, TSG-6 interacts with IαI and forms covalent complexes with HC1 and HC2 of IαI (i.e. HC1⋅TSG-6 and HC2⋅TSG-6). In certain instances, in the presence of HA, the HCs are transferred to HA to form rcHC-HA. In some embodiments, a TSG-6⋅HC1 complex is added to pre-bound PTX3/HA complex to catalyze the transfer of HC1 to HA. In some embodiments, the method comprises first contacting immobilized high molecular weight hyaluronan (HMW HA) with pentraxin 3 (PTX3) under suitable conditions to form a PTX3/HA complex, then contacting the PTX3/HA complex with a HC1⋅TSG-6 complex. In some embodiments, a combination of HC1⋅TSG-6 complex and HC2⋅TSG-6 complex is added to a PTX3/HA complex.

In some embodiments, the step of contacting PTX3 to immobilized HMW HA occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments, the step of contacting PTX3 to immobilized BMW HA occurs for at least 2 hours or longer. In some embodiments, the step of contacting PTX3 to immobilized BMW HA occurs for at least 2 hours. In some embodiments, the step of contacting PTX3 to immobilized HMW HA occurs at 37° C. In some embodiments, the step of contacting PTX3 to immobilized BMW HA occurs in 5 mM MgCl2 in PBS.

In some embodiments, the step of contacting the PTX3/HA complex with IαI and TSG-6 to HA occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments the step of contacting the PTX3/HA complex with a HC1⋅TSG-6 complex and/or a HC2⋅TSG-6 complex occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments the step of contacting the PTX3/HA complex with a HC1⋅TSG-6 complex and/or a HC2⋅TSG-6 complex occurs for at least 2 hours or longer. In some embodiments the step of contacting the PTX3/HA complex with a HC1⋅TSG-6 complex and/or a HC2⋅TSG-6 complex occurs for at least 2 hours. In some embodiments the step of contacting the PTX3/HA complex with a HC1⋅TSG-6 complex and/or a HC1⋅TSG-6 complex occurs at 37° C. In some embodiments the step of contacting the PTX3/HA complex with a HC1⋅TSG-6 complex and/or a HC1⋅TSG-6 complex occurs in 5 mM MgCl2 in PBS.

In some embodiments, the method comprises contacting high molecular weight hyaluronan (HMW HA) with a pentraxin 3 (PTX3) protein, inter-α-inhibitor (IαI) protein comprising heavy chain 1 (HC1) and Tumor necrosis factor α-stimulated gene 6 (TSG-6) simultaneously under suitable conditions to form a HC-HA/PTX3 complex. In some embodiments, the contacting of the HMW HA with PTX3, IαI, and TSG-6 occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments the step of contacting the HMW HA, PTX3, IαI, and TSG-6 occurs at 37° C. In some embodiments the step of contacting the HMW HA, PTX3, IαI, and TSG-6 occurs in 5 mM MgCl₂ in PBS.

In some embodiments, the method comprises contacting high molecular weight hyaluronan (HMW HA) with a pentraxin 3 (PTX3) protein, inter-α-inhibitor (IαI) protein comprising heavy chain 1 (HC1), and Tumor necrosis factor α-stimulated gene 6 (TSG-6) sequentially, in any order, under suitable conditions to form a HC-HA/PTX3 complex. In some embodiments, the contacting of the HMW HA with PTX3, IαI, and TSG-6 occurs for at least 10 minutes, at least 30 minutes, at least 1 hour, at least 2 hours, at least 3 hours, at least 4 hours, at least 5 hours, at least 6 hours, at least 12 hours, or at least 24 hours or longer. In some embodiments the step of contacting the BMW HA, PTX3, IαI, and TSG-6 occurs at 37° C. In some embodiments the step of contacting the HMW HA, PTX3, IαI, and TSG-6 occurs in 5 mM MgCl₂ in PBS.

In some embodiments, the methods for production of an rcHC-HA/PTX3 complex further comprise addition of one or more small leucine rich proteoglycans (SLRPs). In some embodiments, a method for generating reconstituted HC-HA/PTX3 complexes comprises (a) contacting immobilized high molecular weight hyaluronan (HMW HA) with pentraxin 3 (PTX3) under suitable conditions to form a PTX3/HA complex, (b) contacting the PTX3/HA complex with IαI and Tumor necrosis factor-Stimulated Gene-6 (TSG-6) and (c) contacting the PTX3/HA complex with one or more SLRPS. Provided herein are rcHC-HA/PTX3 complexes produced by such method. In some embodiments, TSG-6 catalyzes the transfer of heavy chain 1 (HC1) of inter-α-inhibitor (IαI) to HA. In some embodiments, HC1 of IαI forms a covalent linkage with HA. In some embodiments, the steps (a), (b), and (c) of the method are performed sequentially in order. In some embodiments, the steps (a), (b), and (c) of the method are performed simultaneously. In some embodiments, the step (a) of the method is performed and then steps (b) and (c) of the method are performed sequentially in order. In some embodiments, the step (a) of the method is performed and then steps (b) and (c) of the method are performed simultaneously.

In some embodiments, a method for generating reconstituted HC-HA/PTX3 complexes comprises (a) contacting immobilized high molecular weight hyaluronan (HMW HA) with IαI and TSG-6 to HA to form an HC-HA complex pre-bound to TSG-6, (b) contacting the HC-HA complex with pentraxin 3 (PTX3) and (c) contacting the HC-HA complex with one or more SLRPS under suitable conditions to form an rcHC-HA/PTX3 complex. Provided herein are rcHC-HA/PTX3 complexes produced by such method. In some embodiments, HC1 of IαI forms a covalent linkage with HA. In some embodiments, the method comprises contacting an HC-HA complex pre-bound to TSG-6 with PTX3. In some embodiments, the steps (a), (b), and (c) of the method are performed sequentially in order. In some embodiments, the steps (a), (b), and (c) of the method are performed simultaneously. In some embodiments, the step (a) of the method is performed and then steps (b) and (c) of the method are performed sequentially in order. In some embodiments, the step (a) of the method is performed and then steps (b) and (c) of the method are performed simultaneously.

In some embodiments, the SLRP is selected from among a class I, class II, or class III SLRP. In some embodiments, the SLRP is selected from among class I SLRPs, such as decorin and biglycan. In some embodiments, the small leucine-rich proteoglycan is selected from among class II SLRPs, such as fibromodulin, lumican, PRELP (proline arginine rich end leucine-rich protein), keratocan, and osteoadherin. In some embodiments, the small leucine-rich proteoglycan is selected from among class III SLRPs, such as epipycan and osteoglycin. In some embodiments, the small leucine-rich proteoglycan is selected from among bikunin, decorin, biglycan, and osteoadherin. In some embodiments, the small leucine-rich protein comprises a glycosaminoglycan. In some embodiments, the small leucine-rich proteoglycan comprises keratan sulfate.

PTX3

In some embodiments, PTX3 for use in the methods is isolated from a cell or a plurality of cells (e.g., a tissue extract). Exemplary cells suitable for the expression of PTX3 include, but are not limited to, animal cells including, but not limited to, mammalian cells, primate cells, human cells, rodent cells, insect cells, bacteria, and yeast, and plant cells, including, but not limited to, algae, angiosperms, gymnosperms, pteridophytes and bryophytes. In some embodiments, PTX3 for use in the methods is isolated from a human cell. In some embodiments, PTX3 for use in the methods is isolated from a cell that is stimulated with one or more proinflammatory cytokines to upregulate PTX3 expression. In some embodiments, the proinflammatory cytokine is IL-1 or TNF-α.

In some embodiments, PTX3 for use in the methods is isolated from an amniotic membrane cell. In some embodiments, PTX3 for use in the methods is isolated from an amniotic membrane cell from an umbilical cord. In some embodiments, the amniotic membrane cell is stimulated with or more proinflammatory cytokines to upregulate PTX3 expression. In some embodiments, the proinflammatory cytokine is IL-1 or TNF-α.

In some embodiments, PTX3 for use in the methods is isolated from an umbilical cord cell. In some embodiments, the umbilical cord cell is stimulated with or more proinflammatory cytokines to upregulate PTX3 expression. In some embodiments, the proinflammatory cytokine is IL-1 or TNF-α.

In some embodiments, PTX3 for use in the methods is isolated from an amniotic epithelial cell. In some embodiments, PTX3 for use in the methods is isolated from an umbilical cord epithelial cell. In some embodiments, the amniotic epithelial cell or umbilical cord epithelial cell is stimulated with or more proinflammatory cytokines to upregulate PTX3 expression. In some embodiments, the proinflammatory cytokine is IL-1 or TNF-α.

In some embodiments, PTX3 for use in the methods is isolated from an amniotic stromal cell. In some embodiments, PTX3 for use in the methods is isolated from an umbilical cord stromal cell. In some embodiments, the amniotic stromal cell or umbilical cord stromal cell is stimulated with or more proinflammatory cytokines to upregulate PTX3 expression. In some embodiments, the proinflammatory cytokine is IL-1 or TNF-α.

In some embodiments, PTX3 for use in the methods is a native PTX3 protein isolated from a cell. In some embodiments, the cell is stimulated with or more proinflammatory cytokines to upregulate PTX3 expression. In some embodiments, the proinflammatory cytokine is IL-1 or TNF-α.

In some embodiments, PTX3 is prepared by recombinant technology. In some embodiments, PTX3 is expressed from a recombinant expression vector. In some embodiments, nucleic acid encoding PTX3 is operably linked to a constitutive promoter. In some embodiments, nucleic acid encoding PTX3 is operably linked to an inducible promoter. In some embodiments, PTX3 is expressed in a transgenic animal. In some embodiments, PTX3 is a recombinant protein. In some embodiments, PTX3 is a recombinant protein isolated from a cell. In some embodiments, PTX3 is a recombinant protein produced in a cell-free extract.

In some embodiments, PTX3 is purified from amniotic membrane, umbilical cord, umbilical cord amniotic membrane, chorionic membrane, amniotic fluid, or a combination thereof. In some embodiments, PTX3 is purified from amniotic membrane cells. In some embodiments, the amniotic membrane cell is an amniotic epithelial cell. In some embodiments, the amniotic membrane cell is an umbilical cord epithelial cell. In some embodiments, the amniotic membrane cell is an amniotic stromal cell. In some embodiments, the amniotic membrane cell is an umbilical cord stromal cell. In some embodiments, the amniotic membrane cell is stimulated with or more proinflammatory cytokines to upregulate PTX3 expression. In some embodiments, the proinflammatory cytokine is IL-1 or TNF-α.

In some embodiments, PTX3 is not isolated from a cell or a plurality of cells (e.g., a tissue extract).

In some embodiments, PTX3 comprises a fragment of PTX3 sufficient to bind to HA and facilitate the formation of rcHC-HA/PTX3 complex. Variants of PTX3 for use in the provided methods include species variants, allelic variants, and variants that contain conservative and non-conservative amino acid mutations. In some instances, PTX3 variants further include variants with an amino acid modification that is an amino acid replacement (substitution), deletion, or insertion. In some embodiments, such modification improves one or more properties of the PTX3 polypeptides such as improving the one or more therapeutic properties of the rcHC-HA/PTX3 complex (e.g., anti-inflammatory, anti-immune, anti-angiogenic, anti-scarring, anti-adhesion, regeneration, or other therapeutic activities as described herein).

In some embodiments PTX3 protein is obtained from a commercial source. An exemplary commercial source for PTX3 is, but is not limited to, PTX3 (Catalog No. 1826-TS; R&D Systems, Minneapolis, Minn.).

In some embodiments, the PTX3 protein used in the methods is a multimeric protein. In some embodiments, the PTX3 protein used in the methods is a homomultimer. In some embodiments, the homomultimer is a dimer, trimer, tetramer, hexamer, pentamer, or octamer. In some embodiments, the PTX3 homomultimer is a trimer, tetramer, or octamer. In particular embodiments, the PTX3 homomultimer is an octamer. In some embodiments, the multimerization domain is modified to improve multimerization of the PTX3 protein. In some embodiments, the multimerization domain is replaced with a heterogeneous multimerization domain (e.g., an Fc multimerization domain or leucine zipper) that, when fused to PTX3, improves the multimerization of PTX3.

TSG-6

In some embodiments, TSG-6 for use in the methods is isolated from a cell or a plurality of cells (e.g., a tissue extract). Exemplary cells suitable for the expression of TSG-6 include, but are not limited to, animal cells including, but not limited to, mammalian cells, primate cells, human cells, rodent cells, insect cells, bacteria, and yeast, and plant cells, including, but not limited to, algae, angiosperms, gymnosperms, pteridophytes and bryophytes. In some embodiments, TSG-6 for use in the methods is isolated from a human cell. In some embodiments, TSG-6 for use in the methods is isolated from a cell that is stimulated with one or more proinflammatory cytokines to upregulate TSG-6 expression. In some embodiments, the proinflammatory cytokine is IL-1 or TNF-α.

In some embodiments, TSG-6 for use in the methods is isolated from an amniotic membrane cell. In some embodiments, TSG-6 for use in the methods is isolated from an amniotic membrane cell from an umbilical cord. In some embodiments, TSG-6 for use in the methods is isolated from an amniotic membrane cell that is stimulated with one or more proinflammatory cytokines to upregulate TSG-6 expression. In some embodiments, the proinflammatory cytokine is IL-1 or TNF-α.

In some embodiments, TSG-6 for use in the methods is isolated from an umbilical cord cell. In some embodiments, TSG-6 for use in the methods is isolated from an umbilical cord cell that is stimulated with one or more proinflammatory cytokines to upregulate TSG-6 expression. In some embodiments, the proinflammatory cytokine is IL-1 or TNF-α.

In some embodiments, TSG-6 for use in the methods is isolated from an amniotic epithelial cell. In some embodiments, TSG-6 for use in the methods is isolated from an umbilical cord epithelial cell. In some embodiments, TSG-6 for use in the methods is isolated from an amniotic epithelial cell or an umbilical cord epithelial cell that is stimulated with one or more proinflammatory cytokines to upregulate TSG-6 expression. In some embodiments, the proinflammatory cytokine is IL-1 or TNF-α.

In some embodiments, TSG-6 for use in the methods is isolated from an amniotic stromal cell. In some embodiments TSG-6 for use in the methods is isolated from an umbilical cord stromal cell. In some embodiments, TSG-6 for use in the methods is isolated from an amniotic stromal cell or an umbilical cord stromal cell that is stimulated with one or more proinflammatory cytokines to upregulate TSG-6 expression. In some embodiments, the proinflammatory cytokine is IL-1 or TNF-α.

In some embodiments, TSG-6 for use in the methods is a native TSG-6 protein isolated from a cell. In some embodiments, the cell is stimulated with or more proinflammatory cytokines to upregulate TSG-6 expression. In some embodiments, the proinflammatory cytokine is IL-1 or TNF-α.

In some embodiments, TSG-6 is prepared by recombinant technology. In some embodiments, TSG-6 is expressed from a recombinant expression vector. In some embodiments, nucleic acid encoding TSG-6 is operably linked to a constitutive promoter. In some embodiments, nucleic acid encoding TSG-6 is operably linked to an inducible promoter. In some embodiments, TSG-6 is expressed in a transgenic animal. In some embodiments, TSG-6 is a recombinant protein. In some embodiments, TSG-6 is a recombinant protein isolated from a cell. In some embodiments, TSG-6 is a recombinant protein produced in a cell-free extract.

In some embodiments, TSG-6 is purified from amniotic membrane, amniotic membrane, chorionic membrane, amniotic fluid, or a combination thereof. In some embodiments, PTX3 is purified from amniotic membrane cells. In some embodiments, the amniotic membrane cell is an amniotic epithelial cell. In some embodiments, the amniotic epithelial cell is an umbilical cord epithelial cell. In some embodiments, the amniotic membrane cell is an amniotic stromal cell. In some embodiments, the amniotic membrane cell is an umbilical cord stromal cell. In some embodiments, the amniotic membrane cell is stimulated with or more proinflammatory cytokines to upregulate TSG-6 expression. In some embodiments, the proinflammatory cytokine is IL-1 or TNF-α.

In some embodiments, TSG-6 is not isolated from a cell or a plurality of cells (e.g., a tissue extract).

In some embodiments, TSG-6 comprises a fragment of TSG-6 that is sufficient to facilitate or catalyze the transfer HC1 of IαI to HA. In some embodiments, TSG-6 comprises the link module of TSG-6. In some embodiments, TSG-6 comprises amino acids Trp18 through Leu277 of TSG-6. In some embodiments, TSG-6 variants include, for example, species variants, allelic variants, and variants that contain conservative and non-conservative amino acid mutations. Natural allelic variants of human TSG-6 include, for example, TSG-6 containing the amino acid replacement Q144R. Variants of TSG-6 or HA binding fragments thereof for use in the provided methods include variants with an amino acid modification that is an amino acid replacement (substitution), deletion, or insertion. In some embodiments, such modification improve one or more properties of the TSG-6 polypeptides such as improved transfer of HC1 of IαI to HA or improved release of the TSG-6 polypeptide from the rcHC-HA/PTX3 complex following transfer of HC1 of IαI to HA.

In some embodiments, TSG-6 comprises an affinity tag. Exemplary affinity tags include, but are not limited to, a hemagglutinin tag, a poly-histidine tag, a myc tag, a FLAG tag, a glutathione-S-transferase (GST) tag. Such affinity tags are well known in the art for use in purification. In some embodiments, such an affinity tag incorporated into the TSG-6 polypeptide as a fusion protein or via a chemical linker. In some embodiments, TSG-6 comprises an affinity tag and the unbound TSG-6 is removed from the rcHC-HA/PTX3 complex by affinity purification.

In some embodiments TSG-6 protein is obtained from a commercial source. An exemplary commercial source for TSG-6 is, but is not limited to, TSG-6 (Catalog No. 2104-TS R&D Systems, Minneapolis, Minn.).

IαI

In some embodiments, the IαI comprises an HC1 chain. In some embodiments, the IαI comprises an HC1 and an HC2 chain. In some embodiments, the IαI comprises an HC1 and bikunin. In some embodiments, the IαI comprises an HC1, and HC2 chain, and bikunin. In some embodiments, the IαI comprises an HC1, and HC2 chain, and bikunin linked by a chondroitin sulfate chain.

In some embodiments, IαI is isolated from a biological sample. In some embodiments the biological sample is a biological sample from a mammal. In some embodiments, the mammal is a human. In some embodiments, the biological sample is a blood, serum, plasma, liver, amniotic membrane, chorionic membrane, or amniotic fluid sample. In some embodiments, the biological sample is a blood, serum, or plasma sample. In some embodiments, the biological sample is a blood sample. In some embodiments, the biological sample is a serum sample. In some embodiments, the biological sample is a plasma sample. In some embodiments, the IαI is purified from human blood, plasma or serum. In some embodiments, IαI is isolated from human serum. In some embodiments, IαI is not isolated from serum. In some embodiments, IαI for use in the methods is produced in an amniotic membrane cell. In some embodiments, IαI for use in the methods is produced in an umbilical cord cell. In some embodiments, IαI for use in the methods is produced in an amniotic membrane cell from an umbilical cord. In some embodiments, IαI for use in the methods is produced in an amniotic epithelial cell. In some embodiments, IαI for use in the methods is produced in an umbilical cord epithelial cell. In some embodiments, IαI for use in the methods is produced in an amniotic stromal cell. In some embodiments, IαI for use in the methods is produced in an umbilical cord stromal cell. In some embodiments, IαI for use in the methods is produced in a hepatic cell. In some embodiments, IαI is prepared by recombinant technology.

In some embodiments, HC1 of IαI is isolated from a biological sample. In some embodiments the biological sample is a biological sample from a mammal. In some embodiments, the mammal is a human. In some embodiments, the biological sample is a blood, serum, plasma, liver, amniotic membrane, chorionic membrane or amniotic fluid sample. In some embodiments, the biological sample is a blood, serum, or plasma sample. In some embodiments, the biological sample is a blood sample. In some embodiments, the biological sample is a serum sample. In some embodiments, the biological sample is a plasma sample. In some embodiments, the HC1 of IαI is purified from human blood, plasma or serum. In some embodiments, IαI is isolated from human serum. In some embodiments, HC1 of IαI is not purified from serum. In some embodiments, HC1 of IαI is prepared by recombinant technology. In some embodiments, HC1 of IαI is purified from hepatic cells. In some embodiments, HC1 of IαI is purified from amniotic membrane cells. In some embodiments, HC1 of IαI is purified from amniotic epithelial cells or umbilical cord epithelial cells. In some embodiments, HC1 of IαI is purified from amniotic stromal cells or umbilical cord stromal cells.

In some embodiments, HC2 of IαI is isolated from a biological sample. In some embodiments the biological sample is a biological sample from a mammal. In some embodiments, the mammal is a human. In some embodiments, the biological sample is a blood, serum, plasma, liver, amniotic membrane, chorionic membrane or amniotic fluid sample. In some embodiments, the biological sample is a blood, serum, or plasma sample. In some embodiments, the biological sample is a blood sample. In some embodiments, the biological sample is a serum sample. In some embodiments, the biological sample is a plasma sample. In some embodiments, the HC2 of IαI is purified from human blood, plasma, or serum. In some embodiments, HC2 of IαI is isolated from human serum. In some embodiments, HC2 of IαI is isolated from human serum. In some embodiments, HC2 of IαI is not isolated from blood serum. In some embodiments, HC2 of IαI is prepared by recombinant technology. In some embodiments, HC2 of IαI is purified from hepatic cells. In some embodiments, HC2 of IαI is purified from amniotic membrane cells. In some embodiments, HC2 of IαI is purified from amniotic epithelial cells or umbilical cord epithelial cells. In some embodiments, HC2 of IαI is purified from amniotic stromal cells or umbilical cord stromal cells.

Hyaluronic Acid (HA)

In some embodiments, HA is purified from a cell, tissue, or a fluid sample. In some embodiments, HA is obtained from a commercial supplier (e.g., Sigma Aldrich or Advanced Medical Optics, Irvine, Calif. (e.g., Healon)). In some embodiments, HA is obtained from a commercial supplier as a powder. In some embodiments, HA is expressed in a cell. Exemplary cells suitable for the expression of HA include, but are not limited to, animal cells including, but not limited to, mammalian cells, primate cells, human cells, rodent cells, insect cells, bacteria, and yeast, and plant cells, including, but not limited to, algae, angiosperms, gymnosperms, pteridophytes and bryophytes. In some embodiments, HA is expressed in a human cell. In some embodiments, HA is expressed in a transgenic animal. In some embodiments, HA is obtained from a cell that expresses a hyaluronan synthase (e.g., HAS1, HAS2, and HAS3). In some embodiments, the cell contains a recombinant expression vector that expresses an HA synthase. In certain instances, an HA synthase lengthens hyaluronan by repeatedly adding glucuronic acid and N-acetylglucosamine to the nascent polysaccharide as it is extruded through the cell membrane into the extracellular space.

HA for use in the methods is typically high molecular weight (HMW) HA. In some embodiments, the weight average molecular weight of HMW HA is greater than about 100 kilodaltons (kDa), such as, for example, between about 100 kDa and about 10,000 kDa, between about 500 kDa and about 10,000 kDa, between about 800 kDa and about 8,500 kDa, between about 1100 kDa and about 5,000 kDa, or between about 1400 kDa and about 3,500 kDa. In some embodiments, the weight average molecular weight of HMW HA is about 3000 kDa.

Additional Components

In some embodiments, one or more additional components are added to generate an rcHC-HA/PTX3 complex. In some embodiments, a small leucine rich proteoglycan (SLRP) is added to generate an rcHC-HA/PTX3 complex. In some embodiments, the SLRP is a class I, class II or class III SLRP. In some embodiments, the SLRP is selected from among class I SLRPs, such as decorin and biglycan. In some embodiments, the SLRP is selected from among class II SLRPs, such as fibromodulin, lumican, PRELP (proline arginine rich end leucine-rich protein), keratocan, and osteoadherin. In some embodiments, the SLRP is selected from among class III SLRPs, such as epipycan and osteoglycin. In some embodiments, the SLRP is selected from among bikunin, decorin, biglycan, and osteoadherin. In some embodiments, the SLRP comprises a glycosaminoglycan. In some embodiments, the SLRP comprises keratan sulfate.

HA Immobilization

In some embodiments, HMW HA is immobilized by any suitable method. In some embodiments, HMW HA is immobilized to a solid support, such as culture dish, bead, a column or other suitable surfaces, such as, for example, a surface of an implantable medical device or a portion thereof or on a surface that is subsequently connected to or combined with an implantable medical device as described herein. In some embodiments, HMW HA is immobilized directly to the solid support, such a by chemical linkage. In some embodiments, HMW HA is attached indirectly to the solid support via a linker or an intermediary protein. Numerous heterobifunctional cross-linking reagents that are used to form covalent bonds between amino groups and thiol groups and to introduce thiol groups into proteins, are known to those of skill in this art. In some embodiments, HMW HA is immobilized directly to the solid support via crosslinking to the solid support. In some embodiments, HMW HA is immobilized directly to the solid support without crosslinking to the solid support. In some embodiments, HMW HA is immobilized directly to the solid support as a coating. In some embodiments, HMW HA is immobilized to a Covalink™-NH surface. In some embodiments, BMW HA is immobilized directly to the solid support as a coating. In some embodiments, BMW HA is immobilized to a Covalink™-NH surface for about 16 h at 4° C.

In some embodiments, the method comprises immobilizing BMW HA to a solid surface via direct linkage to a solid support (i.e. without an intermediary protein). In some embodiments, the solid support is washed to remove unbound HMW HA prior to contacting the immobilized HA with PTX3. In some embodiments, the solid support is washed with washes of 8M GnHCl and PBS to remove unbound HMW HA prior to contacting the immobilized HA with PTX3.

In some embodiments, the method comprises immobilizing HA to a solid surface via an intermediary protein or a linker. In some embodiments, the linker is a peptide linker. In some embodiments, the intermediary protein is an HA binding protein (HABP). In some embodiments, HABP is first attached to a solid support (e.g., by cross-linking, chemical linkage or via a chemical linker). In some embodiments, the solid support comprising HABP is then contacted with HA (e.g., BMW HA) to immobilize HA to the solid support via binding of the HABP to HA. In some embodiments, the solid support is washed to remove unbound BMW HA prior to contacting the immobilized HMW HA with PTX3. In some embodiments, the solid support is washed with washes of 8M GnHCl and PBS to remove unbound BMW HA prior to contacting the immobilized HA with PTX3.

In some embodiments, the method comprises immobilizing HA to a solid surface via attachment of a peptide linker to the solid support and attachment HA to the peptide linker. In some embodiments, the peptide linker comprises a protease cleavage site.

In some embodiments, the method comprises immobilizing HA to a solid surface via attachment of a cleavable chemical linker, such as, but not limited to a disulfide chemical linker.

In some embodiments, the HABP selected for use in the methods is an HABP that is dissociated from HA following formation of the rcHC-HA/PTX3 complex. In some embodiments, the HABP non-covalently binds to HA. In some embodiments, the method further comprises dissociating the rcHC-HA/PTX3 complex from HABP using one or more dissociating agents. Dissociating agents for the disruption of non-covalent interactions (e.g., guanidine hydrochloride, urea and various detergents, e.g., SDS) are known in the art. In some embodiments the dissociating agent is urea. In some embodiments the dissociating agent is guanidine hydrochloride. In some embodiments, the dissociation agent is about 4M to about 8M guanidine-HCl. In some embodiments, the dissociation agent is about 4M, about 5M, about 6M, about 7M, about 8M guanidine-HCl. In some embodiments, the dissociation agent is about 4M to about 8M guanidine-HCl in PBS at pH 7.5.

In some embodiments, such dissociating agents are employed to dissociate the rcHC-HA/PTX3 complex from an intermediary HABP. An HABP for use in the methods typically is selected such that the binding affinity for HA is strong enough to permit assembly of the rcHC-HA/PTX3 complex but is dissociated from the rcHC-HA/PTX3 complex with a suitable dissociation agent. In some embodiments the dissociating agent is guanidine hydrochloride.

Exemplary HABPs for use with the methods provided herein include, but are not limited to, HAPLN1, HAPLN2, HAPLN3, HAPLN4, aggrecan, versican, neurocan, brevican, phosphacan, TSG-6, CD44, stabilin-1, stabilin-2, or portions thereof (e.g., link modules thereof) sufficient to bind HA. In some embodiments, the HABP is versican. In some embodiments, the HABP is a recombinant protein. In some embodiments, the HABP is a recombinant mammalian protein. In some embodiments, the HABP is a recombinant human protein. In some embodiments, the HABP is a recombinant versican protein or a portion thereof sufficient to bind to HA. In some embodiments, the HABP is a recombinant aggrecan protein or a portion thereof sufficient to bind to HA. In some embodiments, the HABP is a native HABP or a portion thereof sufficient to bind to HA. In some embodiments, the native HABP is isolated from mammalian tissue or cells. In some embodiments, the HABP is isolated from bovine nasal cartilage (e.g. HABP from Seikagaku which contains the HA binding domains of aggrecan and link protein).

In some embodiments, the HABP comprises a link module of HAPLN1, HAPLN2, HAPLN3, HAPLN4, aggrecan, versican, neurocan, brevican, phosphacan, TSG-6, CD44, stabilin-1, or stabilin-2. In some embodiments, the HABP comprises a link module of versican. In some embodiments, the HABP comprising a link module is a recombinant protein. In some embodiments, the HABP comprising a link module of versican is a recombinant protein.

In some embodiments, the or intermediary protein, such as an HABP, contains a proteolytic cleavage sequence that is recognized by and is hydrolyzed by a site specific protease, such as furin, 3C protease, caspase, matrix metalloproteinase, or TEV protease. In such embodiments, assembled rcHC-HA/PTX3 complexes are released from the solid support by contacting the immobilized complexes with a protease that cleaves the specific cleavage sequence.

In some embodiments, the rcHC-HA/PTX3 complex is purified. In some embodiments, the rcHC-HA/PTX3 complex is purified by any suitable method or combination of methods. The embodiments described below are not intended to be exclusive, only exemplary.

In some embodiments, the rcHC-HA/PTX3 complex is purified by chromatography (e.g., ion exchange, affinity, size exclusion, and hydroxyapatite chromatography), gel filtration, centrifugation (e.g., gradient centrifugation), or differential solubility, ethanol precipitation, or by any other available technique for the purification of proteins.

In some embodiments, the rcHC-HA/PTX3 complex is purified by immunoaffinity chromatography. In some embodiments antibodies are generated against a component of the rcHC-HA/PTX3 complex (e.g., anti-HC1, anti-PTX, and an antibody against one or more SLRPs of the rcHC-HA/PTX3 complex, e.g., anti-bikunin, anti-decorin, anti-biglycan, or anti-osteoadherin) and affixed to a solid support. In some embodiments, the unpurified rcHC-HA/PTX3 complex (i.e., the mobile phase) is passed over the support. In certain instances, the rcHC-HA/PTX3 complex binds to the antibodies. In some embodiments, the support is washed (e.g., with PBS) to remove any unbound or loosely bound molecules. In some embodiments, the support is then washed with a solution that enables elution of the rcHC-HA/PTX3 complex from the support (e.g., 1% SDS, 6M guanidine-HCl, or 8M urea). In some embodiments, the dissociating agent is removed from the dissociated rcHC-HA/PTX3 complex. In some embodiments, the dissociating agent is removed from the dissociated rcHC-HA/PTX3 complex by a method including, but not limited to, ion-exchange chromatography, dialysis, gel filtration chromatography, ultrafiltration, or diafiltration.

In some embodiments, the rcHC-HA/PTX3 complex is purified by affinity chromatography. In some embodiments, an HABP is employed to bind to the rcHC-HA/PTX3 complex for purification of the complex and affixed to a stationary support. In some embodiments, the unpurified rcHC-HA/PTX3 complex (i.e., the mobile phase) is passed over the support. In certain instances, the rcHC-HA/PTX3 complex binds to the HABP. In some embodiments the support is washed (e.g., with PBS) to remove any unbound or loosely bound molecules. In some embodiments, the support is then washed with a solution (e.g., a dissociating agent) that enables elution of the rcHC-HA/PTX3 complex from the support. In some embodiments, the dissociating agent is removed from the dissociated rcHC-HA/PTX3 complex by a method including, but not limited to, ion-exchange chromatography, dialysis, gel filtration chromatography, ultrafiltration, or diafiltration.

In some embodiments, the rcHC-HA/PTX3 complex is purified by a combination of HABP affinity chromatography, and immunoaffinity chromatography using antibodies against one or more components of the rcHC-HA/PTX3 complex.

In some embodiments, one or more components of the rcHC-HA/PTX3 complex disclosed herein comprise an affinity tag (e.g., a fusion protein of PTX3 or HC1 with an affinity tag). Exemplary affinity tags that are incorporated into one or more components of the rcHC-HA/PTX3 complex in some embodiments include, but are not limited to, a hemagglutinin tag, poly-histidine, a myc tag, a FLAG tag, or glutathione-S-transferase sequence. In some embodiments, the ligand for the affinity tag is affixed to the solid support. In some embodiments, the unpurified rcHC-HA/PTX3 complex is passed over the support. In certain instances, the rcHC-HA/PTX3 complex binds to the ligand. In some embodiments the support is washed (e.g., with PBS) to remove any unbound or loosely bound molecules. In some embodiments, the support is then washed with a solution that enables elution of an rcHC-HA/PTX3 complex disclosed herein from the support. In some embodiments, the elution agent is removed from the dissociated rcHC-HA/PTX3 complex by a method including, but not limited to, ion-exchange chromatography, dialysis, gel filtration chromatography, ultrafiltration, or diafiltration.

In some embodiments, the PTX3, TSG-6, and/or HC1 are conjugated to a label. A “label” refers to a detectable compound or composition which is conjugated directly or indirectly to a polypeptide so as to generate a labeled polypeptide. In some embodiments, the label is detectable by itself (e.g., radioisotope labels or fluorescent labels) or, in the case of an enzymatic label, catalyzes chemical alteration of a substrate compound composition which is detectable. Non-limiting examples of labels include fluorogenic moieties, dyes, fluorescent tags, green fluorescent protein, or luciferase.

### Pharmaceutical Compositions

In some embodiments, a preparation comprising HC-HA/PTX3 is a pharmaceutical composition. In some embodiments, the HC-HA/PTX3 complexes are nHC-HA/PTX3 or rcHC-HA/PTX3 complexes, as described herein. In some embodiments, the pharmaceutical composition consists essentially of an nHC-HA/PTX3 complex or an rcHC-HA/PTX3 complex. In some embodiments, the pharmaceutical composition comprise a pharmaceutically acceptable diluent, excipient, vehicle, or carrier. In some embodiments, proper formulation of the pharmaceutical composition is dependent upon the route of administration selected. Any of the well-known techniques, carriers, and excipients can be used as suitable and as understood in the art.

In some embodiments, the pharmaceutical composition further comprises at least one pharmaceutically acceptable carrier. In some embodiments, the pharmaceutical composition further comprises an adjuvant, excipient, preservative, agent for delaying absorption, filler, binder, adsorbent, buffer, and/or solubilizing agent. Exemplary pharmaceutical compositions that are formulated to comprise an HC-HA/PTX3 complex provided herein include, but are not limited to, a gel, solution, suspension, emulsion, syrup, granule, powder, homogenate, ointment, tablet, capsule, pill or an aerosol. In some embodiments, the preparation comprising HC-HA/PTX3 is a graft or a sheet.

In some embodiments, the pharmaceutical composition further comprises a therapeutic cell. In some embodiments, the therapeutic cell is a progenitor cell, a stem cell, or an induced pluripotent stem cell. In some embodiments, the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell.

Dosage Forms

In some embodiments, a preparation comprising an HC-HA/PTX3 complex is administered as an aqueous suspension. In some embodiments, an aqueous suspension comprises water, Ringer's solution and/or isotonic sodium chloride solution. In some embodiments, an aqueous suspension comprises a sweetening or flavoring agent, coloring matters or dyes and, if desired, emulsifying agents or suspending agents, together with diluents water, ethanol, propylene glycol, glycerin, or combinations thereof. In some embodiments, an aqueous suspension comprises a suspending agent. In some embodiments, an aqueous suspension comprises sodium carboxymethylcellulose, methylcellulose, hydroxypropylmethyl-cellulose, sodium alginate, polyvinyl-pyrrolidone, gum tragacanth and/or gum acacia. In some embodiments, an aqueous suspension comprises a dispersing or wetting agent. In some embodiments, an aqueous suspension comprises a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. In some embodiments, an aqueous suspension comprises a preservative. In some embodiments, an aqueous suspension comprises ethyl, or n-propyl p-hydroxybenzoate. In some embodiments, an aqueous suspension comprises a sweetening agent. In some embodiments, an aqueous suspension comprises sucrose, saccharin or aspartame.

In some embodiments, a preparation comprising an HC-HA/PTX3 complex is administered as an oily suspension. In some embodiments, an oily suspension is formulated by suspending the active ingredient in a vegetable oil (e.g., arachis oil, olive oil, sesame oil or coconut oil), or in mineral oil (e.g., liquid paraffin). In some embodiments, an oily suspension comprises a thickening agent (e.g., beeswax, hard paraffin or cetyl alcohol). In some embodiments, an oily suspension comprises sweetening agents (e.g., those set forth above). In some embodiments, an oily suspension comprises an anti-oxidant (e.g., butylated hydroxyanisol or alpha-tocopherol).

In some embodiments, a preparation comprising an HC-HA/PTX3 complex is formulated for parenteral injection (e.g., via injection or infusion, including intraarterial, intraarticular, intracardiac, intradermal, intraduodenal, intramedullary, intramuscular, intraosseous, intraperitoneal, intrathecal, intravascular, intravenous, intravitreal, epidural and/or subcutaneous). In some embodiments, the preparation comprising an HC-HA/PTX3 complex is administered as a sterile solution, suspension or emulsion.

In some embodiments, a formulation for parenteral administration includes aqueous and/or non-aqueous (oily) sterile injection solutions of a preparation comprising an HC-HA/PTX3 complex, which in some embodiments, contain antioxidants, buffers, bacteriostats and/or solutes which render the formulation isotonic with the blood of the intended recipient; and/or aqueous and/or non-aqueous sterile suspensions which in some embodiments, include a suspending agent and/or a thickening agent. In some embodiments, a formulation for parenteral administration includes suitable stabilizers or agents which increase the solubility of a preparation comprising an HC-HA/PTX3 complex to allow for the preparation of highly concentrated solutions.

In some embodiments, a preparation comprising an HC-HA/PTX3 complex is administered as an oil-in-water micro-emulsion where the active ingredient is dissolved in the oily phase. In some embodiments, a preparation comprising an HC-HA/PTX3 complex is dissolved in a fatty oil (e.g., sesame oil, or synthetic fatty acid esters, (e.g., ethyl oleate or triglycerides, or liposomes. In some embodiments, a preparation comprising an HC-HA/PTX3 complex disclosed herein is dissolved in a mixture of soybean oil and/or lecithin. In some embodiments, the oil solution is introduced into a water and glycerol mixture and processed to form a micro-emulsion.

In some embodiments, a composition formulated for parenteral administration is administered as a single bolus shot. In some embodiments, a composition formulated for parenteral administration is administered via a continuous intravenous delivery device (e.g., Deltec CADD-PLUS™ model 5400 intravenous pump).

In some embodiments, a formulation for injection is presented in unit dosage form, e.g., in ampoules or in multi-dose containers, with an added preservative. In some embodiments, a formulation for injection is stored in powder form or in a freeze-dried (lyophilized) condition requiring only the addition of the sterile liquid carrier, for example, saline or sterile pyrogen-free water, immediately prior to use.

In some embodiments, a preparation comprising an HC-HA/PTX3 complex is formulated for topical administration. Topical formulations include, but are not limited to, ointments, creams, lotions, solutions, pastes, gels, films, sticks, liposomes, nanoparticles. In some embodiments, a topical formulation is administered by use of a patch, bandage or wound dressing.

In some embodiments, a preparation comprising an HC-HA/PTX3 complex is formulated as composition is in the form of a solid, a cross-linked gel, or a liposome. In some embodiments, preparation comprising an HC-HA/PTX3 complex is formulated as an insoluble cross-linked hydrogel.

In some embodiments, a topical formulation comprises a gelling (or thickening) agent. Suitable gelling agents include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, acacia (gum arabic), agar, aluminum magnesium silicate, sodium alginate, sodium stearate, bladderwrack, bentonite, carbomer, carrageenan, carbopol, xanthan, cellulose, microcrystalline cellulose (MCC), ceratonia, chondrus, dextrose, furcellaran, gelatin, ghatti gum, guar gum, hectorite, lactose, sucrose, maltodextrin, mannitol, sorbitol, honey, maize starch, wheat starch, rice starch, potato starch, gelatin, sterculia gum, polyethylene glycol (e.g. PEG 200-4500), gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), or combinations thereof.

In some embodiments, a topical formulation disclosed herein comprises an emollient. Emollients include, but are not limited to, castor oil esters, cocoa butter esters, safflower oil esters, cottonseed oil esters, corn oil esters, olive oil esters, cod liver oil esters, almond oil esters, avocado oil esters, palm oil esters, sesame oil esters, squalene esters, kikui oil esters, soybean oil esters, acetylated monoglycerides, ethoxylated glyceryl monostearate, hexyl laurate, isohexyl laurate, isohexyl palmitate, isopropyl palmitate, methyl palmitate, decyloleate, isodecyl oleate, hexadecyl stearate decyl stearate, isopropyl isostearate, methyl isostearate, diisopropyl adipate, diisohexyl adipate, dihexyldecyl adipate, diisopropyl sebacate, lauryl lactate, myristyl lactate, and cetyl lactate, oleyl myristate, oleyl stearate, and oleyl oleate, pelargonic acid, lauric acid, myristic acid, palmitic acid, stearic acid, isostearic acid, hydroxystearic acid, oleic acid, linoleic acid, ricinoleic acid, arachidic acid, behenic acid, erucic acid, lauryl alcohol, myristyl alcohol, cetyl alcohol, hexadecyl alcohol, stearyl alcohol, isostearyl alcohol, hydroxystearyl alcohol, oleyl alcohol, ricinoleyl alcohol, behenyl alcohol, erucyl alcohol, 2-octyl dodecanyl alcohol, lanolin and lanolin derivatives, beeswax, spermaceti, myristyl myristate, stearyl stearate, carnauba wax, candelilla wax, lecithin, and cholesterol.

In some embodiments, a preparation comprising an HC-HA/PTX3 complex is formulated with one or more natural polymers. In some embodiments, a preparation comprising an HC-HA/PTX3 complex is formulated with a natural polymer that is fibronectin, collagen, laminin, keratin, fibrin, fibrinogen, hyaluronic acid, heparan sulfate, chondroitin sulfate. In some embodiments, a preparation comprising an HC-HA/PTX3 complex is formulated with a polymer gel formulated from a natural polymer. In some embodiments, a preparation comprising an HC-HA/PTX3 complex is formulated with a polymer gel formulated from a natural polymer, such as, but not limited to, fibronectin, collagen, laminin, keratin, fibrin, fibrinogen, hyaluronic acid, heparan sulfate, chondroitin sulfate, and combinations thereof. In some embodiments, a preparation comprising an HC-HA/PTX3 complex is formulated with a cross-linked polymer. In some embodiments, a preparation comprising an HC-HA/PTX3 complex is formulated with a non-cross-linked polymer. In some embodiments, a preparation comprising an HC-HA/PTX3 complex is formulated with a non-cross-linked polymer and a cross-linked polymer. In some embodiments, a preparation comprising an HC-HA/PTX3 complex is formulated with cross-linked hyaluronan gel. In some embodiments, a preparation comprising an HC-HA/PTX3 complex is formulated with an insoluble cross-linked HA hydrogel. In some embodiments, a preparation comprising an HC-HA/PTX3 complex is formulated with non-cross-linked hyaluronan gel. In some embodiments, a preparation comprising an HC-HA/PTX3 complex is formulated with a collagen matrix. In some embodiments, a preparation comprising an HC-HA/PTX3 complex is formulated with a fibrin matrix. In some embodiments, a preparation comprising an HC-HA/PTX3 complex is formulated with a fibrin/collagen matrix.

In some embodiments, a preparation comprising an HC-HA/PTX3 complex is formulated for administration to an eye or a tissue related thereto. Formulations suitable for administration to an eye include, but are not limited to, solutions, suspensions (e.g., an aqueous suspension), ointments, gels, creams, liposomes, niosomes, pharmacosomes, nanoparticles, or combinations thereof. In some embodiments, a preparation comprising an HC-HA/PTX3 complex for topical administration to an eye is administered spraying, washing, or combinations thereof. In some embodiments, a preparation comprising an HC-HA/PTX3 complex is administered to an eye via an injectable depot preparation.

As used herein, a “depot preparation” is a controlled-release formulation that is implanted in an eye or a tissue related thereto (e.g., the sclera) (for example subcutaneously, intramuscularly, intravitreally, or within the subconjunctiva). In some embodiments, a depot preparation is formulated by forming microencapsulated matrices (also known as microencapsulated matrices) of a preparation comprising an HC-HA/PTX3 complex in biodegradable polymers. In some embodiments, a depot preparation is formulated by entrapping a preparation comprising an HC-HA/PTX3 complex in liposomes or microemulsions.

A formulation for administration to an eye has an ophthalmically acceptable tonicity. In certain instances, lacrimal fluid has an isotonicity value equivalent to that of a 0.9% sodium chloride solution. In some embodiments, an isotonicity value from about 0.6% to about 1.8% sodium chloride equivalency is suitable for topical administration to an eye. In some embodiments, a formulation for administration to an eye disclosed herein has an osmolarity from about 200 to about 600 mOsm/L. In some embodiments, a formulation for administration to an eye disclosed herein is hypotonic and thus requires the addition of any suitable to attain the proper tonicity range. Ophthalmically acceptable substances that modulate tonicity include, but are not limited to, sodium chloride, potassium chloride, sodium thiosulfate, sodium bisulfate and ammonium sulfate.

A formulation for administration to an eye has an ophthalmically acceptable clarity. Examples of ophthalmically-acceptable clarifying agents include, but are not limited to, polysorbate 20, polysorbate 80, or combinations thereof.

In some embodiments, a formulation for administration to an eye comprises an ophthalmically acceptable viscosity enhancer. In some embodiments, a viscosity enhancer increases the time a formulation disclosed herein remains in an eye. In some embodiments, increasing the time a formulation disclosed herein remains in the eye allows for greater drug absorption and effect. Non-limiting examples of mucoadhesive polymers include carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, a formulation for administration to an eye is administered or delivered to the posterior segments of an eye (e.g., to the retina, choroid, vitreous and optic nerve). In some embodiments, a topical formulation for administration to an eye disclosed herein for delivery to the posterior of the eye comprises a solubilizing agent, for example, a glucan sulfate and/or a cyclodextrin. Glucan sulfates which are used in some embodiments include, but are not limited to, dextran sulfate, cyclodextrin sulfate and β-1,3-glucan sulfate, both natural and derivatives thereof, or any compound which temporarily binds to and be retained at tissues which contain fibroblast growth factor (FGF), which improves the stability and/or solubility of a drug, and/or which improves penetration and ophthalmic absorption of a topical formulation for administration to an eye disclosed herein. Cyclodextrin derivatives which are used in some embodiments as a solubilizing agent include, but are not limited to, α-cyclodextrin, β-cyclodextrin, γ-cyclodextrin, hydroxyethyl β-cyclodextrin, hydroxypropyl γ-cyclodextrin, hydroxypropyl β-cyclodextrin, sulfated α-cyclodextrin, sulfated β-cyclodextrin, sulfobutyl ether β-cyclodextrin.

In some embodiments, a preparation comprising an HC-HA/PTX3 complex is formulated for rectal or vaginal administration. In some embodiments, a preparation comprising an HC-HA/PTX3 complex is administered as a suppository. In some embodiments, a composition suitable for rectal administration is prepared by mixing a preparation comprising an HC-HA/PTX3 complex with a suitable non-irritating excipient which is solid at ordinary temperatures but liquid at the rectal temperature and will therefore melt in the rectum to release the drug. In some embodiments, a composition suitable for rectal administration is prepared by mixing a preparation comprising an HC-HA/PTX3 complex with cocoa butter, glycerinated gelatin, hydrogenated vegetable oils, mixtures of polyethylene glycols of various molecular weights or fatty acid esters of polyethylene glycol.

In some embodiments, a preparation comprising an HC-HA/PTX3 complex is formulated for inhalation. In some embodiments, the preparation is in a nebulizer, a pressurized metered-dose inhaler (pMDI), or a dry-powder inhaler (DPI).

In certain embodiments, a preparation comprising an HC-HA/PTX3 complex is optionally incorporated within controlled release particles, lipid complexes, liposomes, nanoparticles, microspheres, microparticles, nanocapsules or other agents which enhance or facilitate localized delivery to the skin. An example of a conventional microencapsulation process for pharmaceutical preparations is described in U.S. Pat. No. 3,737,337, incorporated herein by reference for such disclosure.

Dosages

The amount of pharmaceutical compositions administered is dependent in part on the individual being treated. In instances where pharmaceutical compositions are administered to a human subject, the daily dosage will normally be determined by the prescribing physician with the dosage generally varying according to the age, sex, diet, weight, general health, and response of the individual, the severity of the individual's symptoms, the precise disease or condition being treated, the severity of the disease or condition being treated, time of administration, route of administration, the disposition of the composition, rate of excretion, drug combination, and the discretion of the prescribing physician.

In some embodiments, the dosage of a preparation comprising an HC-HA/PTX3 complex is between about 0.001 to about 1000 mg/kg body weight/day. In some embodiments, the amount of a preparation comprising an HC-HA/PTX3 complex is in the range of about 0.5 to about 50 mg/kg/day. In some embodiments, the amount of nHC-HA/PTX3 or rcHC-HA/PTX3 complex disclosed herein is about 0.001 to about 7 g/day. In some embodiments, the amount of a preparation comprising an HC-HA/PTX3 complex is about 0.01 to about 7 g/day. In some embodiments, the amount of a preparation comprising an HC-HA/PTX3 complex disclosed herein is about 0.02 to about 5 g/day. In some embodiments, the amount of a preparation comprising an HC-HA/PTX3 complex is about 0.05 to about 2.5 g/day. In some embodiments, the amount of a preparation comprising an HC-HA/PTX3 complex is about 0.1 to about 1 g/day.

In some embodiments, a preparation comprising an HC-HA/PTX3 complex is administered, before, during, or after the occurrence of unwanted changes in a tissue. In some embodiments, a combination therapy is administered before, during, or after the occurrence of unwanted changes in a tissue. In some embodiments, a preparation comprising an HC-HA/PTX3 complex is administered with a combination therapy before, during or after the occurrence of a disease or condition. In some embodiments, the timing of administering the composition containing an nHC-HA/PTX3 or rcHC-HA/PTX3 disclosed herein varies. Thus, in some examples, a preparation comprising an HC-HA/PTX3 complex is used as a prophylactic and is administered continuously to subjects with a propensity to develop unwanted changes in a tissue in order to prevent the occurrence of unwanted changes in the tissue. In some embodiments, a preparation comprising an HC-HA/PTX3 complex is administered to a subject during or as soon as possible after the onset of the unwanted changes. In some embodiments, the administration of a preparation comprising an HC-HA/PTX3 complex is initiated within the first 48 hours of the onset of the unwanted changes, preferably within the first 48 hours of the onset of the symptoms, more preferably within the first 6 hours of the onset of the symptoms, and most preferably within 3 hours of the onset of the symptoms. In some embodiments, the initial administration is via any route practical, such as, for example, an intravenous injection, a bolus injection, infusion over 5 minutes to about 5 hours, a pill, a capsule, transdermal patch, buccal delivery, or combination thereof. A preparation comprising an HC-HA/PTX3 complex is preferably administered as soon as is practicable after the onset of unwanted changes is detected or suspected, and for a length of time necessary for the treatment, such as, for example, from about 1 month to about 3 months. In some embodiments, the length of treatment varies for each subject, and the length is determined using the known criteria. In some embodiments, a preparation comprising an HC-HA/PTX3 complex or a formulation containing a complex is administered for at least 2 weeks, preferably about 1 month to about 5 years, and more preferably from about 1 month to about 3 years.

In some embodiments, a preparation comprising an HC-HA/PTX3 complex is administered in a single dose, once daily. In some embodiments, a preparation comprising an HC-HA/PTX3 complex is administered in multiple doses, more than once per day. In some embodiments, a preparation comprising an HC-HA/PTX3 complex is administered twice daily. In some embodiments, a preparation comprising an HC-HA/PTX3 complex is administered three times per day. In some embodiments, an nHC-HA/PTX3 or rcHC-HA/PTX3 complex is administered four times per day. In some embodiments, a preparation comprising an HC-HA/PTX3 complex is administered more than four times per day.

In some embodiments, a preparation comprising an HC-HA/PTX3 complex is administered for prophylactic and/or therapeutic treatments. In therapeutic applications, in some embodiments, a preparation comprising an HC-HA/PTX3 complex is administered to an individual already suffering from a disease or condition resulting in a tissue having unwanted changes, in an amount sufficient to cure or at least partially arrest the unwanted changes. Amounts effective for this use will depend on the severity and course of the unwanted changes caused by the disease or condition, previous therapy, the individual's health status, weight, and response to the drugs, and the judgment of the treating physician.

In prophylactic applications, in some embodiments, a preparation comprising an HC-HA/PTX3 complex is administered to an individual that is at risk of a particular disorder that may result in the individual having unwanted changes in their tissue. Such an amount is defined to be a “prophylactically effective amount or dose.” In such use, the precise amounts also depend on the individual's state of health, weight, and other physical parameters of the individual.

In the case wherein the individual's condition does not improve, upon the doctor's discretion a preparation comprising an HC-HA/PTX3 complex is administered chronically, that is, for an extended period of time, including throughout the duration of the individual's life in order to ameliorate or otherwise control or limit the symptoms of the individual's disease or condition.

In some embodiments, in cases where the individual's status does improve, upon the doctor's discretion, a preparation comprising an HC-HA/PTX3 complex is administered continuously or the dose of drug being administered is temporarily reduced or temporarily suspended for a certain length of time (i.e., a “drug holiday”). In some embodiments, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. In some embodiments the dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the individual's conditions has occurred, a maintenance dose is administered if necessary. In some embodiments, subsequently, the dosage or the frequency of administration, or both, is reduced, as a function of the symptoms, to a level at which the improved condition is retained. In some embodiments, individuals require intermittent treatment on a long-term basis upon any recurrence of unwanted changes.

In some embodiments, the pharmaceutical composition described herein is in unit dosage forms suitable for single administration of precise dosages. In unit dosage form, the formulation is divided into unit doses containing appropriate quantities of an nHC-HA/PTX3 or rcHC-HA/PTX3 complex disclosed herein. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. In some embodiments, multiple-dose reclosable containers are used, in which case it is typical to include a preservative in the composition. In some embodiments, formulations for parenteral injection are presented in unit dosage form, which include, but are not limited to ampoules, or in multi dose containers, with an added preservative.

The daily dosages appropriate for a preparation comprising an HC-HA/PTX3 complex are, for example, from about 0.01 to 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. In some embodiments, the dosages are altered depending on a number of variables, not limited to the activity of an nHC-HA/PTX3 or rcHC-HA/PTX3 complex, the extent of the unwanted changes in the tissue, the mode of administration, the requirements of the individual subject, the severity of the unwanted changes, and the judgment of the practitioner.

In some embodiments, the toxicity and therapeutic efficacy of such therapeutic regimens are determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). In some embodiments, the dose ratio between the toxic and therapeutic effects is the therapeutic index and it is expressed as the ratio between LD₅₀ and ED₅₀. nHC-HA/PTX3 or rcHC-HA/PTX3 complexes exhibiting high therapeutic indices are preferred. In some embodiments, the data obtained from cell culture assays and animal studies is used in formulating a range of dosages for use in human. The dosage of a preparation comprising an HC-HA/PTX3 complex lies preferably within a range of circulating concentrations that include the ED₅₀ with minimal toxicity. In some embodiments, the dosage varies within this range depending upon the dosage form employed and the route of administration utilized.

In some embodiments, preparations comprising an HC-HA/PTX3 complex are packaged as articles of manufacture containing packaging material, a pharmaceutical composition which is effective for prophylaxis and/or treating a disease or condition, and a label that indicates that the pharmaceutical composition is to be used for reprogramming a fibroblastic cell in a tissue having unwanted changes due to a disease or condition. In some embodiments, the pharmaceutical compositions are packaged in unit dosage forms contain an amount of the pharmaceutical composition for a single dose or multiple doses. In some embodiments, the packaged compositions contain a lyophilized powder of the pharmaceutical compositions, which is reconstituted (e.g., with water or saline) prior to administration.

Medical Device and Biomaterials Compositions

In some embodiments, a preparation comprising an HC-HA/PTX3 complex is assembled directly on a surface of or formulated as a coating for an implantable medical device. Methods for covalent attachment of hyaluronan to surfaces such as, but not limited to, metallic, polymeric, ceramic, silica and composite surfaces is well-known in the art and in some embodiments, is employed in conjunction with the methods provided herein for the assembly of nHC-HA/PTX3 or rcHC-HA/PTX3 complexes on such surfaces (see e.g., U.S. Pat. Nos. 5,356,433; 5,336,518, 4,613,665, 4,810,784, 5,037,677, 8,093,365). In some embodiments, an nHC-HA/PTX3 or rcHC-HA/PTX3 complex is assembled directly on a surface of an implantable medical device or a portion thereof. In some embodiments, an nHC-HA/PTX3 or rcHC-HA/PTX3 complex that has been generated according the methods provided herein is purified and then attached directly on a surface of an implantable medical device or a portion thereof. In some embodiments, an nHC-HA/PTX3 or rcHC-HA/PTX3 complex that has been generated according the methods provided herein is purified and then formulated as a coating for attachment to the medical device or a portion thereof. In some embodiments, the coating is applied directly to the surfaces or is applied to a pretreated or coated surface where the pretreatment or coating is designed to aid adhesion of the coating to the substrate. In some embodiments, an nHC-HA/PTX3 or rcHC-HA/PTX3 complex that has been generated according the methods provided herein is purified and then attached to a medical device or a portion thereof that has been coated with a substance that promotes the attachment of the nHC-HA/PTX3 or rcHC-HA/PTX3 complex. For example, in some embodiments, the medical device or a portion thereof is coated with an adhesive polymer that provides functional groups on its surface for the covalent attachment of hyaluronan of the nHC-HA/PTX3 or rcHC-HA/PTX3 complex. In some embodiments, a coupling agent, such as, but not limited to carbodiimide is employed to attach the nHC-HA/PTX3 or rcHC-HA/PTX3 complex to the polymer coating. In some embodiments, photoimmobilization is employed to covalently attach an nHC-HA/PTX3 or rcHC-HA/PTX3 complex that has been generated according the methods provided herein to medical device or a portion thereof. In some embodiments, an nHC-HA/PTX3 or rcHC-HA/PTX3 complex that has been generated according the methods provided herein is attached to a medical device or a portion thereof using a spacer molecule that comprises a photochemically or thermochemically reactive group.

In some embodiments, the coating formulations comprising an nHC-HA/PTX3 or rcHC-HA/PTX3 complex are applied to the substrate by for example dip-coating. Other methods of application include, but are not limited to, spray, wash, vapor deposition, brush, roller, curtain, spin coating and other methods known in the art.

Exemplary implantable medical devices include, but are not limited to an artificial joint, orthopedic device, bone implant, contact lenses, suture, surgical staple, surgical clip, catheter, angioplasty balloon, sensor, surgical instrument, electrode, needle, syringe, wound drain, shunt, urethral insert, metal or plastic implant, heart valve, artificial organ, lap band, annuloplasty ring, guide wire, K-wire or Denham pin, stent, stent graft, vascular graft, pacemaker, pellets, wafers, medical tubing, infusion sleeve, implantable defibrillator, neurostimulator, glucose sensor, cerebrospinal fluid shunt, implantable drug pump, spinal cage, artificial disc, ocular implant, cochlear implant, breast implant, replacement device for nucleus pulposus, ear tube, intraocular lens, drug delivery system, microparticle, nanoparticle, and microcapsule.

In particular embodiments, the implantable medical device is an implant or prosthesis comprising an nHC-HA/PTX3 or rcHC-HA/PTX3 complex disclosed herein. In particular embodiments, the prosthesis is an artificial joint. In some embodiments, the prosthesis is an artificial hip joint, artificial knee, an artificial glenohumeral joint, an artificial ankle.

In particular embodiments, the implant is a stent. In particular embodiments, the implant is a coronary stent, a ureteral stent, a urethral stent, a prostatic stent, a bone stent, or an esophageal stent. In particular embodiments, the implant is a coronary stent. In particular embodiments, the implant is a bone implant, such as, but not limited to, an osseointegrated implant or a craniofacial prosthesis (e.g., an artificial ear, orbital prosthesis, nose prosthesis).

In some embodiments, a preparation comprising an HC-HA/PTX3 complex is assembled directly on a microparticle or a nanoparticle for delivery of the HC-HA/PTX3 complex (e.g. nHC-HA/PTX3 or rcHC-HA/PTX3) to a subject (see, e.g., WO 03/015755 and US2004/0241248).

In some embodiments, the preparation comprising an HC-HA/PTX3 complex provided herein are attached to, assembled on, or provided as a coating on the surfaces of or portions thereof of any such implantable medical devices as described herein or known in the art. In some embodiments the preparation comprising an HC-HA/PTX3 complex elutes from the coating and into the surrounding tissue following implantation.

In some embodiments, a preparation comprising an HC-HA/PTX3 complex is assembled directly on a scaffold, a microparticle, a microcapsule or microcarrier employed for the delivery of a biomaterial, such as a stem cell or an insulin producing cell. In some embodiments, a preparation comprising an HC-HA/PTX3 complex is attached to the microcapsule or assembled directly on a microcapsule. In some embodiments, the preparation comprising an HC-HA/PTX3 complex is combined with a material used to form the microcapsule and a microcapsule is generated that contains the preparation comprising an HC-HA/PTX3 complex. In some embodiments, the preparation comprising an HC-HA/PTX3 complex is used to coat the inner surface of the microcapsule. In some embodiments, the preparation comprising an HC-HA/PTX3 complex is used to coat the outer surface of the microcapsule. In some embodiments, the preparation comprising an HC-HA/PTX3 complex is used to coat the inner and outer surface of the microcapsule.

Exemplary materials for encapsulating cells include, but are not limited to, thermosetting hydrogels, such as agarose, alginate, and artificial polymers such as poly(NiPAAm-co-AAC), poly(ethylene glycol) (PEG) and PEG derivatives such as PEG diacrylate and oligo(poly(ethylene glycol)) fumerate. Methods for the culturing and microencapsulation of stem cells are known in the art in some embodiments, are employed to generate microcapsules containing a preparation comprising an HC-HA/PTX3 complex provided herein.

In some embodiments the microcapsule contains a cell, a plurality of cells or other biological material. In some embodiments, the cell or cells are stem cells, such as, but not limited to, mesenchymal stem cells. In some embodiments, the cell or cells are differentiated cells, such as, but not limited to, insulin-producing cells. In some embodiments, the cell or cells are autologous cells (i.e. cells that are from or derived from the recipient of the cells). In some embodiments, the cell or cells are allogeneic cells (i.e. cells that are not from or derived from the recipient of the cells). In some embodiments, the microcapsule contains a cell, a plurality of cells or other biological material and the inner surfaces of the microcapsule are coated with a preparation comprising an HC-HA/PTX3 complex provided herein. In some embodiments the microcapsule contains a cell, a plurality of cells or other biological material and the outer surfaces of the microcapsule are coated with a preparation comprising an HC-HA/PTX3 complex provided herein. In some embodiments the microcapsule contains a cell, a plurality of cells or other biological material and the outer and inner surfaces of the microcapsule are coated with a preparation comprising an HC-HA/PTX3 complex provided herein. In some embodiments the microcapsule is administered to reprogram a fibroblastic cell in a tissue having unwanted changes due to a disease or condition. Exemplary diseases and conditions and methods of treatment for which a microcapsule can be administered are described elsewhere herein and include but are not limited to inflammatory and immune related diseases.

### Methods of Use

Provided herein, in certain embodiments, are uses of HC-HA/PTX3, including preparations or compositions comprising HC-HA/PTX3, to reprogram the cellular phenotype of a cell into a different cellular phenotype. Such reprogramming is used in methods provided herein of, for example, reversing a diseased or damaged state of a tissue (e.g., a damaged or scarred tissue, or a tissue affected by a disease such as a degenerative disease); reprogramming a differentiated cell in a tissue to a progenitor cell, thereby rejuvenating the tissue; reprogramming a first phenotype of a cell in a tissue to a progenitor cell, and differentiating the progenitor cell into a second phenotype, thereby regenerating the tissue. Also provided herein are uses of HC-HA/PTX3, including preparations or compositions comprising HC-HA/PTX3, in compositions with therapeutic cells.

Disclosed herein, in some embodiments, are methods of reversing a disease state in a tissue comprising contacting the tissue with HC-HA/PTX3 or a pharmaceutical composition comprising HC-HA/PTX3 for a time sufficient to reprogram diseased or unwanted cells in the tissue a cell having a different phenotype, thereby reversing the disease state of the tissue. In some embodiments, the cell having the different phenotype is a progenitor cell. In some embodiments, the cell having the different phenotype is an earlier cell in a cellular differentiation pathway.

Disclosed herein, in some embodiments, are methods of reverting a cell in a cellular differentiation pathway to an earlier cell in the cellular differentiation pathway, the method comprising contacting the cell with HC-HA/PTX3 or a pharmaceutical composition comprising HC-HA/PTX3, wherein the contacting occurs for a time sufficient to revert the cell to the earlier cell.

Further disclosed herein, in some embodiments, are methods of treating a condition characterized by unwanted fibroblastic cell differentiation in a subject in need thereof comprising, contacting a fibroblastic cell within a tissue affected by the condition in the subject with HC-HA/PTX3 or a pharmaceutical composition comprising HC-HA/PTX3 for a period of time sufficient to revert the fibroblastic cell to an earlier cell in a cellular differentiation pathway, thereby treating the condition. In some embodiments, the condition occurs as the result of a burn, a laceration, ischemic tissue, a wound, an injury, an ulcer, radiation, chemotherapy, or a surgical incision. In some embodiments, the condition is myocardial infarction.

In some embodiments, the contacting is within a period of time following an injury to the cell or a tissue comprising the cell. In some embodiments, the period of time is less than 1 hour, 2 hours, 3 hours, 4 hours, 5 hours, 6 hours, 7 hours, 8 hours, 9 hours, 10 hours, 11 hours, 12 hours, 24 hours, 36 hours, 2 days, 3 days, 4 days, 5 days, 6 days, 7 days, 2 weeks, 3 weeks, or 4 weeks. following the injury to the cell or the tissue. In some embodiments, the contacting occurs during a surgical procedure. In some embodiments, the surgical procedure comprises placement of a stent.

In some instances, the tissue is not a scar tissue. In some instances, the tissue is a scar tissue.

In some instances, the unwanted fibroblastic cells comprise fibroblasts generated by degenerative disease, aging, scarring, wound, burn, surgical incision, laceration, ulceration, injury, or ischemia. In some embodiments, an unwanted fibroblastic cell is a fibroblastic cell that has undergone differentiation into a cell type characteristic of a degenerative disease, aging, scarring, wound, burn, surgical incision, laceration, ulceration, injury, or ischemia, wherein the differentiation does not occur in the absence of the degenerative disease, aging, scarring, wound, burn, surgical incision, laceration, ulceration, injury, or ischemia. For example, in some embodiments, the unwanted fibroblast is a myofibroblast. In some instances, the unwanted fibroblastic cells comprise fibroblasts and myofibroblasts generated by degenerative disease, aging, scarring, wound, burn, surgical incision, laceration, ulceration, injury, or ischemia. In some embodiments, the fibroblastic cell is a dermal fibroblast. In some embodiments, the fibroblastic cell is a corneal fibroblast. In some embodiments, the fibroblastic cell is a cardiac fibroblast. In some embodiments, the fibroblastic cell is a myofibroblast. In some instances, the fibroblastic cell is not a myofibroblast differentiated from an amniotic membrane stromal cell.

In some instances, the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof. In some instances, the preparation is an extract of fetal support tissue. In some instances, the preparation is a fetal support tissue homogenate. In some instances, the preparation is a fetal support tissue powder. In some instances, the preparation is a morselized fetal support tissue. In some instances, the preparation is a pulverized fetal support tissue. In some instances, the preparation is a ground fetal support tissue. In some instances, the preparation is a fetal support tissue graft. In some instances, the preparation is a purified HC-HA/PTX3. In some instances, the preparation is a reconstituted HC-HA/PTX3.

In some instances, the fetal support tissue is selected from placenta, placental amniotic membrane, umbilical cord, umbilical cord amniotic membrane, chorion, amnion-chorion, amniotic stroma, amniotic jelly, or a combination thereof. In some instances, the fetal support tissue is from placenta. In some instances, the fetal support tissue is from placental amniotic membrane. In some instances, the fetal support tissue is from umbilical cord. In some instances, the fetal support tissue is from umbilical cord amniotic membrane. In some instances, the fetal support tissue is from chorion. In some instances, the fetal support tissue is from amnion-chorion. In some instances, the fetal support tissue is from amniotic stroma. In some instances, the fetal support tissue is from amniotic jelly.

In some instances, the fetal support tissue is frozen or previously frozen. In some instances, the fetal support tissue is substantially free of red blood cells. In some instances, the fetal support tissue comprises umbilical cord substantially free of a vein or artery. In some instances, the fetal support tissue comprises cells, substantially all of which are dead. In some instances, the fetal support tissue comprises umbilical cord amniotic membrane and at least a portion of Wharton's Jelly. In some instances, fetal support tissue is cryopreserved, lyophilized, sterilized, or a combination thereof. In some instances, fetal support tissue is cryopreserved. In some instances, fetal support tissue is lyophilized. In some instances, fetal support tissue is sterilized.

In some instances, the composition is a gel, a solution, or a suspension. In some instances, the composition is a gel. In some instances, the composition is a solution. In some instances, the composition is a suspension.

In some instances, the HC-HA/PTX3 is native HC-HA/PTX3, reconstituted HC-HA/PTX3, or a combination thereof. In some instances, the HC-HA/PTX3 is native HC-HA/PTX3. In some instances, the HC-HA/PTX3 is reconstituted HC-HA/PTX3

In some instances, the tissue having unwanted changes is ocular, cardiac, skin, joint, spine, soft tissue, cartilage, bone, tendon, ligament, nerve, muscle tissue, intervertebral disc, spinal cord, or brain. In some instances, the tissue having unwanted changes is an ocular tissue. In some instances, the tissue having unwanted changes is a cardiac tissue. In some instances, the tissue having unwanted changes is a skin tissue. In some instances, the tissue having unwanted changes is a joint tissue. In some instances, the tissue having unwanted changes is from a spine. In some instances, the tissue having unwanted changes is a soft tissue. In some instances, the tissue having unwanted changes is a cartilage. In some instances, the tissue having unwanted changes is a bone. In some instances, the tissue having unwanted changes is a tendon. In some instances, the tissue having unwanted changes is a ligament. In some instances, the tissue having unwanted changes is a nerve. In some instances, the tissue having unwanted changes is a muscle tissue. In some instances, the tissue having unwanted changes is an intervertebral disc. In some instances, the tissue having unwanted changes is a spinal cord. In some instances, the tissue having unwanted changes is a brain. In some instances, the tissue comprises degenerated tissue, a burn, a laceration, ischemic tissue, a wound, an injury, an ulcer, or a surgical incision. In some instances, the tissue comprises a degenerated tissue. In some instances, the tissue comprises a burn. In some instances, the tissue comprises a laceration. In some instances, the tissue comprises a ischemic tissue. In some instances, the tissue comprises a wound. In some instances, the tissue comprises an injury. In some instances, the tissue comprises an ulcer. In some instances, the tissue comprises a surgical incision. In some instances, the injury is a myocardial infarction.

In some instances, the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell. In some instances, the progenitor cell is a neural crest progenitor. In some instances, the progenitor cell is a hematopoietic progenitor cell. In some instances, the progenitor cell is a mammary progenitor cell. In some instances, the progenitor cell is a intestinal progenitor cell. In some instances, the progenitor cell is a mesenchymal progenitor cell. In some instances, the progenitor cell is an endothelial progenitor cell. In some instances, the progenitor cell is a neural progenitor cell. In some instances, the progenitor cell is an olfactory progenitor cell. In some instances, the progenitor cell is a testicular progenitor cell. In some instances, the progenitor cell is a cardiovascular progenitor cell. In some embodiments, the contacting occurs in vivo.

In some instances, the methods further comprise contacting the fibroblastic cell with TGFβ1. In some embodiments, additional administration of TGFβ1 is required to perform the methods described herein. In some embodiments, additional administration of TGFβ1 is not required to perform the methods described herein. In some embodiments, the cell is contacted simultaneously with the preparation comprising HC-HA/PTX3 and TGFβ1. In some embodiments, the cell is contacted sequentially with the preparation comprising HC-HA/PTX3 first and then the TGFβ1. In some embodiments, the cell is contacted sequentially with the TGFβ1 first and then the preparation comprising HC-HA/PTX3. In some embodiments, the TGFβ1 is administered in a therapeutically effective amount. In some embodiments, a therapeutically effective amount of TGFβ1 is an amount of TGFβ1 sufficient to enable the preparation comprising HC-HA/PTX3 to perform the methods described herein.

Also disclosed herein, in some embodiments, are methods of producing a progenitor cell from a differentiated cell comprising contacting the differentiated cell with HC-HA/PTX3 for a time sufficient to reprogram the differentiated cell to a progenitor cell phenotype. In some embodiments, the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell. In some embodiments, the differentiated cell is a limbal niche cell, endothelial cell, keratocyte, fibroblast, or myofibroblast.

Also disclosed herein, in some embodiments, are in vitro methods of producing a progenitor cell, comprising: contacting a culture of fibroblastic cells with a composition comprising: (a) a preparation comprising HC-HA/PTX3; and (b) a pharmaceutically acceptable diluent, excipient, vehicle, or carrier, for a time sufficient to reprogram the fibroblastic cells to a progenitor cells. In some instances, the preparation is an acellular extract of fetal support tissue, a cell culture matrix, purified HC-HA/PTX3, reconstituted HC-HA/PTX3 or a combination thereof. In some instances, the preparation is an acellular extract of fetal support tissue. In some instances, the preparation is a cell culture matrix. In some instances, the preparation is a purified HC-HA/PTX3. In some instances, the preparation is a reconstituted HC-HA/PTX3.

In some instances, the fetal support tissue is selected from placenta, placental amniotic membrane, umbilical cord, umbilical cord amniotic membrane, chorion, amnion-chorion, amniotic stroma, amniotic jelly, or a combination thereof. In some instances, the fetal support tissue is placenta. In some instances, the fetal support tissue is placental amniotic membrane. In some instances, the fetal support tissue is umbilical cord. In some instances, the fetal support tissue is umbilical cord amniotic membrane. In some instances, the fetal support tissue is chorion. In some instances, the fetal support tissue is amnion-chorion. In some instances, the fetal support tissue is amniotic stroma. In some instances, the fetal support tissue is amniotic jelly.

In some instances, the fetal support tissue is frozen or previously frozen. In some instances, the fetal support tissue is substantially free of red blood cells. In some instances, the fetal support tissue comprises umbilical cord substantially free of a vein or artery. In some instances, the fetal support tissue comprises cells, substantially all of which are dead. In some instances, the fetal support tissue comprises umbilical cord amniotic membrane and at least a portion of Wharton's Jelly. In some instances, fetal support tissue is cryopreserved, lyophilized, sterilized, or a combination thereof. In some instances, fetal support tissue is cryopreserved. In some instances, fetal support tissue is lyophilized. In some instances, fetal support tissue is sterilized.

In some instances, the HC-HA/PTX3 is native HC-HA/PTX3, reconstituted HC-HA/PTX3, or a combination thereof. In some instances, the HC-HA/PTX3 is native HC-HA/PTX3. In some instances, the HC-HA/PTX3 is reconstituted HC-HA/PTX3.

In some instances, the fibroblastic cell is a fibroblast, a myofibroblast, a dermal fibroblast, a corneal fibroblast, or a cardiac fibroblast. In some instances, the fibroblastic cell is a fibroblast. In some instances, the fibroblastic cell is a myofibroblast. In some instances, the fibroblastic cell is a dermal fibroblast. In some instances, the fibroblastic cell is a corneal fibroblast. In some instances, the fibroblastic cell is a cardiac fibroblast. In some instances, the fibroblastic cell is a human corneal fibroblast.

In some instances, the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell. In some instances, the progenitor cell is a neural crest progenitor. In some instances, the progenitor cell is a hematopoietic progenitor cell. In some instances, the progenitor cell is a mammary progenitor cell. In some instances, the progenitor cell is an intestinal progenitor cell. In some instances, the progenitor cell is a mesenchymal progenitor cell. In some instances, the progenitor cell is an endothelial progenitor cell. In some instances, the progenitor cell is a neural progenitor cell. In some instances, the progenitor cell is an olfactory progenitor cell. In some instances, the progenitor cell is a testicular progenitor cell. In some instances, the progenitor cell is a cardiovascular progenitor cell.

In some instances, the methods further comprise contacting the fibroblastic cell with TGFβ1. In some embodiments, additional contacting with TGFβ1 is required to perform the methods described herein. In some embodiments, additional contacting with TGFβ1 is not required to perform the methods described herein. In some embodiments, the cell is contacted simultaneously with the preparation comprising HC-HA/PTX3 and TGFβ1. In some embodiments, the cell is contacted sequentially with the preparation comprising HC-HA/PTX3 first and then the TGFβ1. In some embodiments, the cell is contacted sequentially with the TGFβ1 first and then the preparation comprising HC-HA/PTX3.

### Examples

### Example 1: Reprogramming of Human Corneal Fibroblasts (HCF) into Neural Crest Progenitors by HC-HA/PTX3 with TGFβ1

### Materials and Methods

### HCF Isolation and Culture

A total of 89 human corneas from individuals aged 18-76 years and maintained at 4° C. in Optisol (Chiron Vision, Irvine, Calif.) for less than 7 days after death were obtained from the Florida Lions Eye Bank (Miami, Fla.) and handled according to the declaration of Helsinki. HCF were isolated and cultured. Epithelial and endothelial cells were removed from corneas, the stroma was cut into cubes of approximately 1 mm³, incubated in 2 mg/ml collagenase for 16 h at 37° C., and then placed in a culture medium consisting of Dulbecco's modified Eagle's medium (DMEM) supplemented with 10% fetal bovine serum containing 50 mg/ml gentamicin and 1.25 mg/ml amphotericin B. The culture medium was changed twice a week. The morphology of the cells was monitored by Nikon Eclipse TS 100 microscope (Melville, N.Y.). Cells cultured to passage 3 (P3) were used for all experiments.

### Treatment of TGFβ1

Human corneal fibroblasts (P3) were seeded on plastics with or without immobilized HC-HA/PTX3 complex for 72 h in DMEM+10% FBS, then serum starved for 24 h and treated with or without TGFβ1 for 24 h before being harvested for mRNA quantitation or immunostaining. For determination of protein of TGFβ receptor or p75NTR, the cells were treated with or without TGF-β1 for 48 h before collection of protein samples because the protein expression lags behind mRNA expression. For TGF-β1 ELISA, the cells were treated with or without TGF-β1 for 24 h, and then cultured in the fresh medium for another 24 h. The supernatants were collected for TGFβ1 ELISA. For TGFβ2 and TGFβ3 ELISA, the cells were treated with or without TGFβ1 for 48 h. Certain HCF were seeded on glass in DMEM+10% FBS for 24 h, then in DMEM+ITS (insulin-transferrin-selenium) for 24 h, treated with/without PBS or hyaluronic acid (HA) or HC-HA/PTX3±TGFβ1 (10 ng/ml)±Marimastat (10 μM) or ±DAPT (10 μM) or ±both for 0, 5, 15, 30 and 45 minutes before being harvested for immunostaining of CD44-ICD, JNK1, Cyclin D1 and p75NTR and for 5 minutes before being harvested for Western blotting of cytoplasmic and nuclear CD44-ICD, active MT1-MMP, and active γ-secretase after compartmental separation of the cellular components.

To Revert HCF into NC Like Cells and to Differentiate NCs into Endothelial Like Cells

To reprogram HCF into NC like cells, HCF were cultured on HC-HA/PTX3 complex in serum-free DMEM-ITS with or without challenge of TGFβ1 for 48 h to induce neural crest like cells. The neural crest like cells were further cultured in a low-calcium DMEM with 10% FBS for 3 weeks to induce HCECs.

### RNA Extraction, Reverse Transcription and Real-Time PCR

Total RNAs were extracted using RNeasy Mini Kit and were reverse transcribed using High Capacity Reverse Transcription Kit (Applied Biosystems). cDNA obtained was amplified by real-time RT-PCR using specific primer-probe mixtures and DNA polymerase in 7000 Real-time PCR System (Thermo-Fisher Scientific, Carlsbad, Calif.). Real-time RT-PCR profile consisted of 10 minutes of initial activation at 95° C., followed by 40 cycles of 15 seconds denaturation at 95° C., and 1 min annealing and 1 min extension at 60° C. The genuine identity of each PCR product was confirmed by the size determination using 2% agarose gels followed by ethidium bromide staining together with PCR marker according to EC3 Imaging System (BioImaging System, Upland, Calif.).

### ELISA

The Quantikine Human ELISA Kits of TGFβs were used for determination of TGFβs following the manufacturer's instructions (R and D Systems, Minneapolis, Minn.).

### Immunostaining

HCF, induced human corneal endothelial cells (HCEC), and HCEC monolayer cultures were air-dried and fixed in 4% formaldehyde, pH 7.0, for 15 min at room temperature, rehydrated in PBS, incubated with 0.2% Triton X-100 for 15 min, and rinsed three times with PBS for 5 min each. After incubation with 2% BSA to block non-specific staining for 30 min, they were incubated with the desired first antibody (all at 1:50 dilution) for 16 h at 4° C. After three washes with PBS, they were incubated with corresponding Alexa-Fluor-conjugated secondary IgG (all 1:100 dilutions) for 60 min. The samples were then counterstained with Hoechst 33342 and analyzed with Zeiss LSM 700 confocal microscope (Thornhood, N.Y.). Corresponding mouse and rabbit sera were used as negative controls for primary monoclonal and polyclonal antibodies, respectively.

### Immuno-Precipitation

The native CD44 protein was immune-precipitated by Immunoprecipitation Kit (Abcam, ab206996) with CD44 antibody (Abcam, ab157107) following the vendor's instructions.

### Western Blotting

Cell lysates were prepared in radioimmunoprecipitation assay (RIPA) buffer or non-denature lysis buffer and resolved on 4-15% (w/v) gradient acrylamide gels for Western blotting. The protein extracts were transferred to a nitrocellulose membrane, which was then blocked with 5% (w/v) fat-free milk in tris-buffered saline (TBST, 50 mM Tris-HCl, pH 7.5, 150 mM NaCl, 0.05% (v/v) Tween-20), followed by sequential incubation with specific primary antibodies against TGFβRI, TGFβRII, p75NTR, cyclin D1, CD44-ICD, active MT1-MMP and active γ-secretase and their respective secondary antibodies using β-actin or α-tubulin as the loading control. Immunoreactive proteins were detected with Western Lighting Chemiluminesence.

### Statistical Analysis

All summary data were reported as means±s.d. calculated for each group and compared using the Student's unpaired t-test by Microsoft Excel (Microsoft, Redmont, Wash.). Test results were reported as two-tailed P values, where P<0.05 was considered statistically significant.

### Results

### HC-HA/PTX3 Suppressed Canonical TBFβ Signaling and Myofibroblast Differentiation

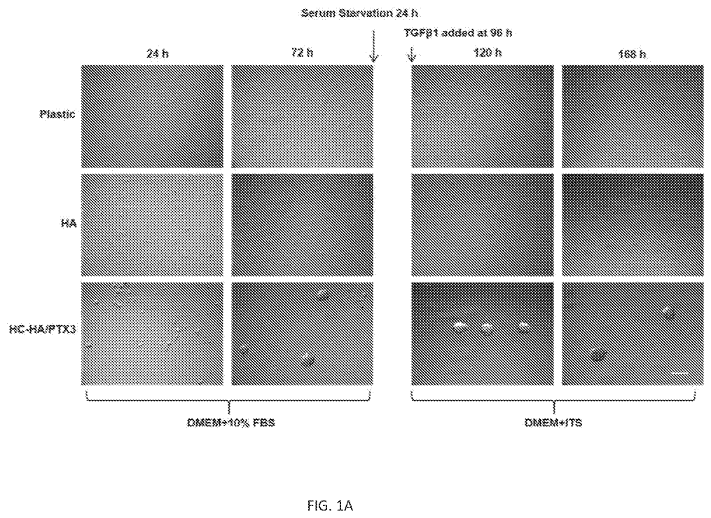

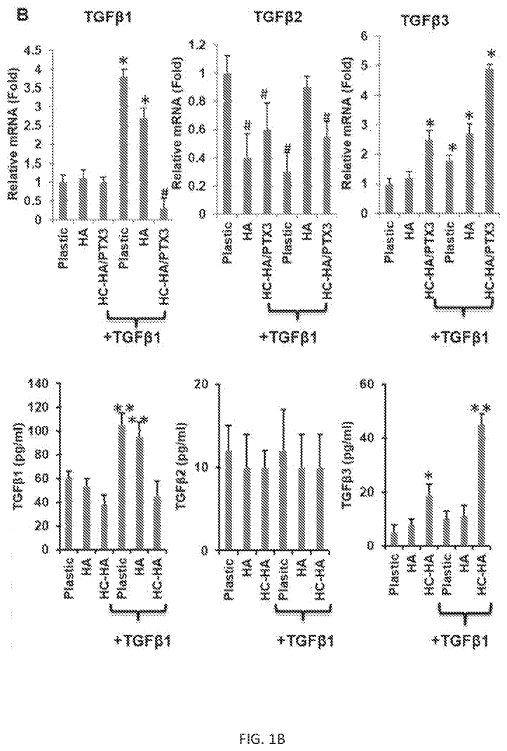

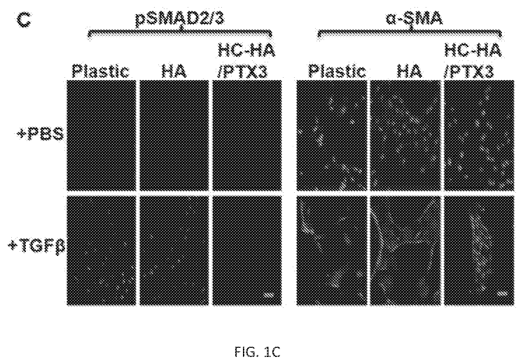

Like the plastic control, Passage 3 (P3) HCF seeded on immobilized HA were spindle in shape. In contrast, with the cells on immobilized HC-HA/PTX3 were aggregated as early as 24 h (FIG. 1A). Spheres were maintained after serum starvation by switching to DMEM+ITS for 24 h despite addition of TGFβ1 for another 72 h (FIG. 1A). The same result was noted in cultures without TGFβ1 (not shown). Using the culture established, exogenous TGFβ1 expectedly upregulated TGFβ1, but not TGFβ2 at both mRNA and protein level, in HCF seeded on both plastic and HA (FIG. 1B). This was not observed on HC-HA/PTX3. Surprisingly, TGFβ3, an anti-scarring isoform, was upregulated at both mRNA and protein level only by HC-HA/PTX3 with or without TGFβ1. As expected, exogenous TGFβ1 caused nuclear translocation of pSMAD2/3 and positive cytoplasmic expression of α-SMA (FIG. 1C) on plastic and HA. Nevertheless, it did not induce such changes in HCF seeded on HC-HA/PTX3 (FIG. 1C).

HC-HA/PTX3 Promoted Expression of Kerotocan in the Absence of TGF-β1, but Expression and Nuclear Translocation of p75NTR in the Presence of TGF-β1, and Expression of NC Markers with and without TGFβ1

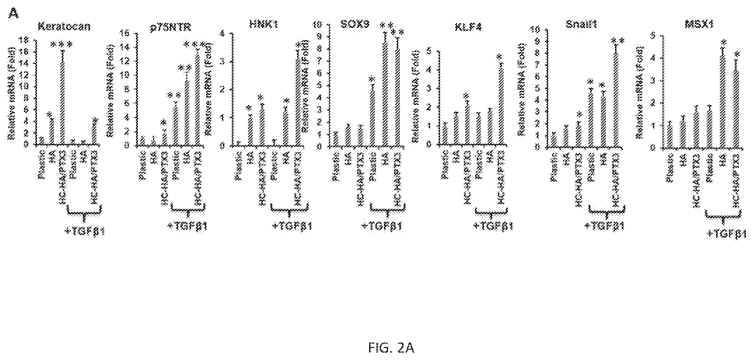

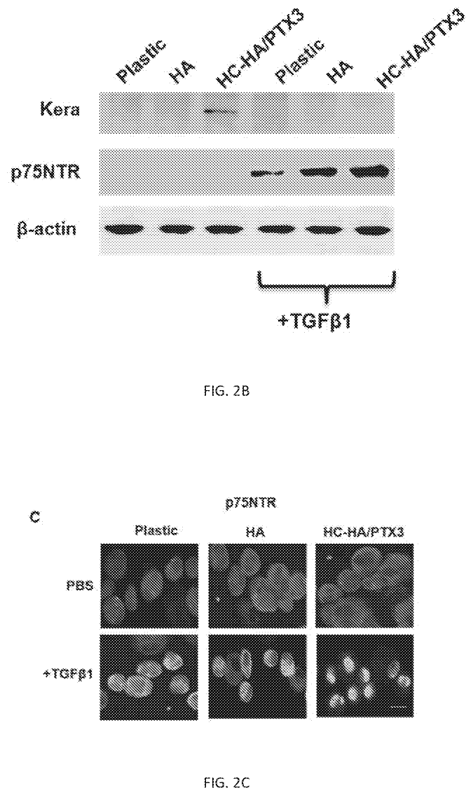

In the absence of TGFβ1, HCF uniquely upregulated both mRNA and protein of keratocan on HC-HA/PTX3 (FIG. 2A). In contrast, HA upregulated mRNA but not protein of keratocan (FIGS. 2A and 2B). In the presence of TGF-β1, the aforementioned upregulation disappeared for both HA and HC-HA/PTX3 (FIG. 2B).

In the absence of TGFβ1, HA only upregulated expression of HNK1 (FIG. 2A). In contrast, HC-HA/PTX3 upregulated expression of all NC markers except Sox9 and MSX1 (FIG. 2A). With TGFβ1, transcript expression of p75NTR, Sox9 and Snail and protein expression of p75 were upregulated on plastic (FIGS. 2A and 2B). mRNA expression p75NTR, HNK1, Sox9, Snail and MSX1 and protein expression of p75NTR was further upregulated without p75NTR nuclear translocation on HA (FIGS. 2A-2C). In contrast, all NC markers and protein of p75NTR were further upregulated with p75NTR nuclear translocation on HC-HA/PTX3 (FIGS. 2A-2C).

Induced NC Progenitors are Verified by Differentiation into Corneal Endothelial Cells

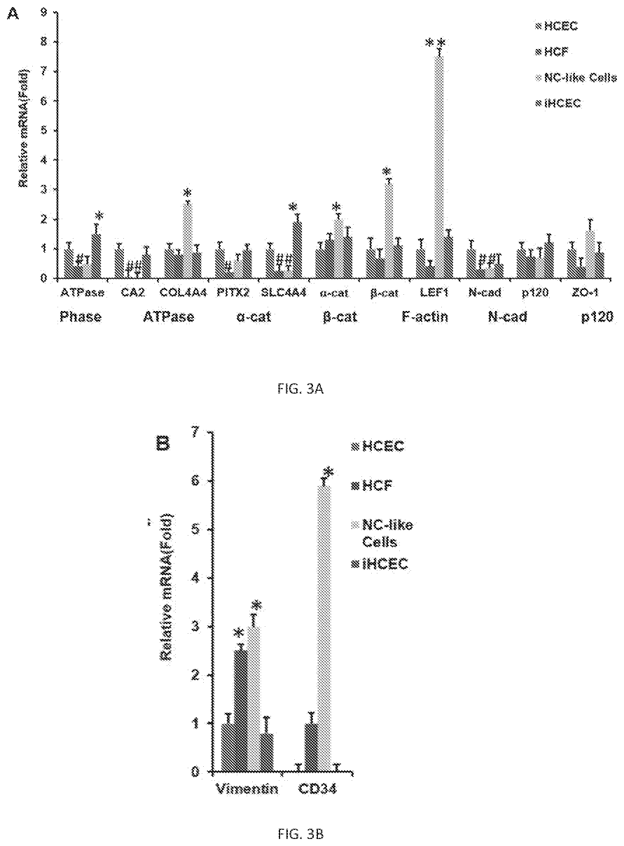

Compared to expression of native HCEC, the mRNA expression of endothelial markers Na—K-ATPase, CA2, COL4A4, PITX2, SLC4A4, LEF1, N-cadherin, ZO-1 was all significantly lower in HCF but the expression of COL4A4, α-catenin, β-catenin, and p120 was similar in HCF (FIG. 3A, #p<0.05, n=3), while the expression of Na—K-ATPase, CA2, SLC4A4, N-cadherin was lower but expression of PITX2, p120 SLC4A4, LEF1, N-cadherin, ZO-1 was similar, and expression of COL4A4, α-catenin, β-catenin, LEF1 was higher in neural crest like cells (FIG. 3A). In addition, induced HCEC expression was similar level of CA2, COL4A4, PITX2, α-catenin, β-catenin, LEF1, p120 and ZO-1, but lower level of N-cadherin and higher level of Na—K-ATPase, SLC4A4 when compared to those from native HCEC (FIG. 3A). In addition, compared to native HCF fibroblastic markers, the expression of vimentin and CD34 was significantly increased in both HCF and induced NC cells but not in induced HCEC (FIG. 3B).

### Unique Downregulation of TGFβRII by Cyclin D1 to Inhibit Canonical TGF Signaling During Reprogramming

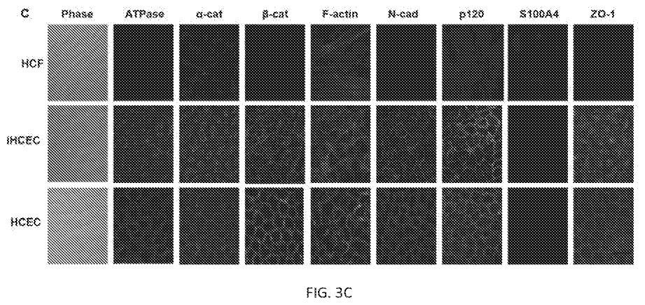

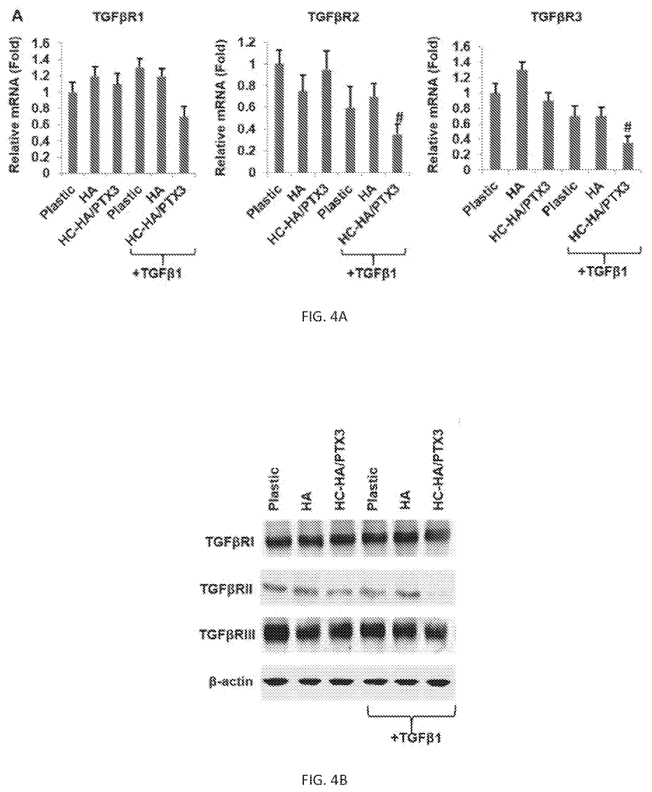

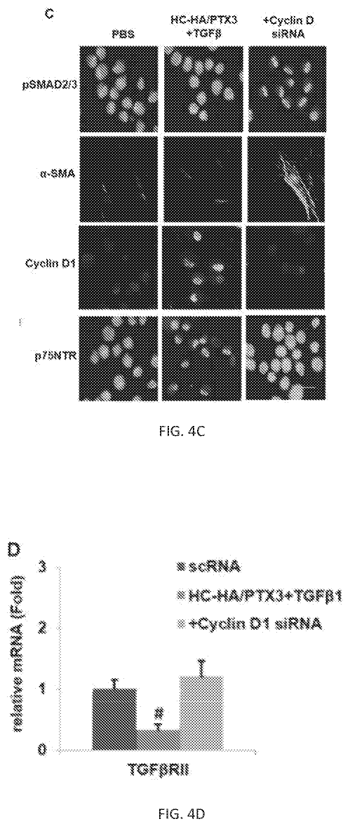

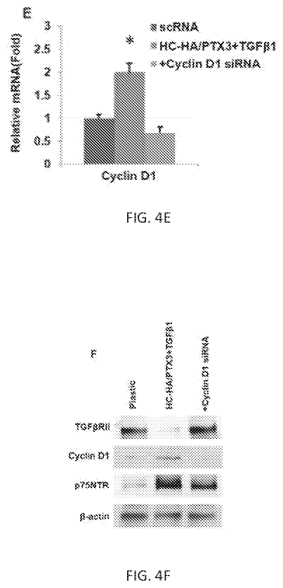

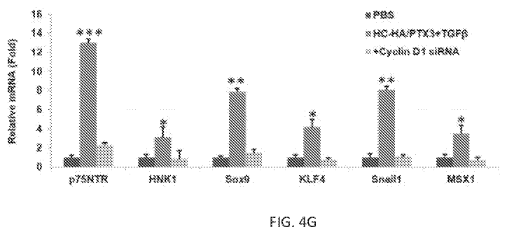

The results showed that expression of TGFβRII mRNA was reduced by 3-fold and expression of TGFβRII protein was reduced to nearly nil on HC-HA/PTX3 after TGFβ1 challenge (FIGS. 4A and 4B). Although expression of TGFβRIII mRNA was reduced by 4-fold, however, expression of TGFβRIII protein was not significantly lower after addition of TGFβ1 (FIGS. 4A and 4B). Therefore, such mRNA reduction may not be significant to affect outcome. The results also showed overexpression of mRNA and protein, and nuclear translocation of Cyclin D1 synergistically promoted by HC-HA/PTX3+TGFβ1 was attenuated by cyclin D1 siRNA (FIG. 4C-4F). Such downregulation of Cyclin D1 by Cyclin D1 siRNA was accompanied by an increase of TGFβRII mRNA, protein (FIG. 4D and FIG. 4F). These results also showed Cyclin D1 siRNA could reverse inhibition of canonical TGFβ signaling since Cyclin D1 siRNA reversed inhibition of nuclear pSMAD2/3 by HC-HA/PTX3+TGFβ1 (FIG. 4C). In addition, Cyclin D1 siRNA was able to reverse inhibition of α-SMA formation by HC-HA/PTX3 (FIG. 4C). Furthermore, nuclear translocation of p75NTR (FIG. 4C), overexpression of p75NTR mRNA and protein (FIG. 4F and FIG. 411), and overexpression of other NC markers induced by HC-HA/PTX3+TGFβ1 (FIG. 411) were all inhibited by Cyclin D1 siRNA.

Sequential activation fCD44-ICD-TAK1-JNK1-cyclin D1-p75NTR by HC-HA/PTX3+TGFβ1

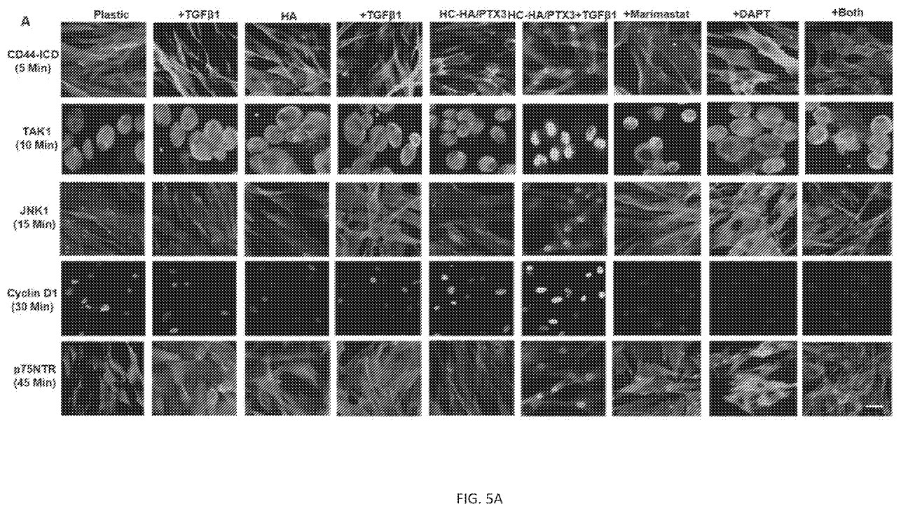

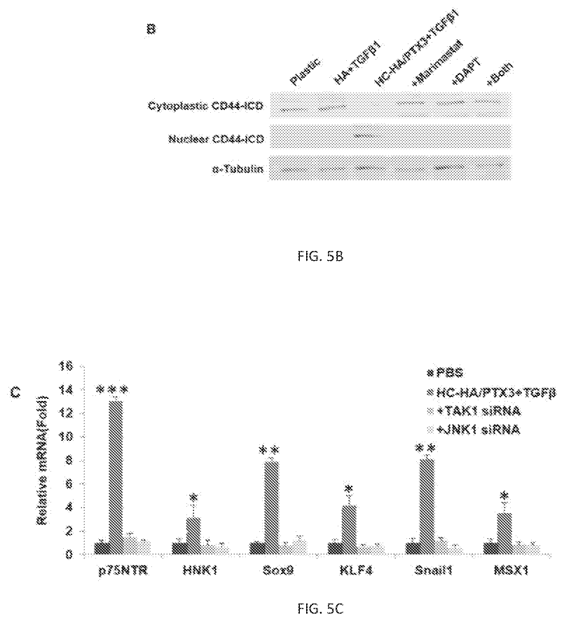

These results showed that only HC-HA/PTX3+TGFβ1 promoted nuclear translocation of CD44-ICD as early as 5 min (FIG. 5A). In additional HC-HA/PTX3+TGFβ1 promoted nuclear translocation of TAK1 at 10 minutes and of JNK1 as early as 15 minutes (FIG. 5A). Furthermore, these results showed that only HC-HA/PTX3+TGFβ1 significantly promoted nuclear translocation of Cyclin D1 as early as 30 minutes (FIG. 5A). Finally, these results showed that HC-HA/PTX3+TGFβ1 promoted nuclear translocation of p75NTR as early as 45 minutes (FIG. 5A). The results suggested the sequential activation of CD44-ICD-TAK1-JNK1-Cyclin D1-p75NTR by HC-HA/PTX3+TGFβ1.

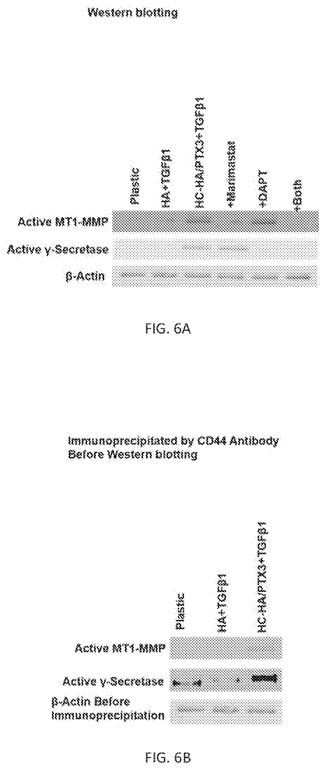

Marimastat, a broad spectrum MMP inhibitor, or DAPT, a specific GSI [γ-secretase inhibitor (GSI)], or both inhibited nuclear translocation of CD44-ICD at 5 min (FIG. 5A). Following this effect by Marimastat or DAPT or both, nuclear translocation of TAK1, JNK1, Cyclin D1 and p75NTR was also inhibited at later time points (FIG. 6A). Consistent with immunostaining results, HC-HA/PTX3+TGFβ1 promoted nuclear translocation of CD44-ICD at 5 minutes by Western blotting (FIG. 6B). Marimastat, a broad spectrum MMP inhibitor, or DAPT, a specific GSI [γ-secretase inhibitor (GSI)], or both inhibited nuclear translocation of CD44-ICD at 5 minutes by Western blotting (FIG. 5B). As the results showed, HC-HA/PTX3+TGFβ1 activated both MT1-MMP and γ-secretase, which were inhibited by their respective inhibitors by Western blotting. The induced NC potential was also associated with activation of TAK1-JNK1 signaling because inhibition of TAK1 and JNK1 by their siRNAs attenuated their upregulation of NC markers by HC-HA/PTX3+TGFβ1 (FIG. 5B).

Activation of MT1-MMP and γ-Secretase was Mediated by Interaction of MT1-MMP and γ-Secretase with CD44

The immune-precipitation and Western blotting results showed that only HC-HA/PTX3+TGFβ1 promoted interaction of CD44 with MT1-MMP and γ-secretase as early as 5 minutes (FIGS. 6A-6B), suggesting that activation of MT1-MMP and γ-secretase was mediated by interaction of MT1-MMP and γ-secretase with CD44.

### Discussion

HCF could be obtained from cadaveric corneal stroma after collagenase digestion and cultured on plastic in DMEM+10% FBS to Passage 3 (P3). P3 HCF seeded on plastic with or without immobilized HA showed normal spindle shape (FIG. 1A). However, P3 HCF formed aggregate on immobilized HC-HA/PTX3 as early as 24 hours in the same medium (FIG. 1A), suggesting change of cell shape from spindle to small round shape. Spheres were maintained after serum starvation by switching to DMEM+ITS for 24 h with or without addition of TGFβ1 for another 72 h (FIG. 1A). Collectively, the results indicated that those cells might change their phenotype on HC-HA/PTX3 to that of younger cells due to the shape change even under TGFβ1 challenge. The results resemble what was reported previously, for example, a small portion of bovine corneal stromal cells exhibit clonal growth and human corneal stromal cells could be expanded clonally in attachment-free cultures as “neutrospheres”, and such “corneal stromal stem cells” exhibited properties of mesenchymal stem cells (MSCs), including clonal growth, multipotent differentiation, and expression of an array of stem cell-specific markers.

Using the culture established above, in the absence of TGFβ1, HCF seeded on HC-HA/PTX3 exhibited no change regarding TGFβ signaling except for upregulation of TGFβ3 (FIG. 1B). Exogenous TGFβ1 expectedly upregulated TGFβ1, but not TGFβ2, in HCF seeded on both plastic and HA but not HC-HA/PTX3, and surprisingly upregulated TGFβ3, an anti-scarring isoform, only by HC-HA/PTX3 with or without TGFβ1 (FIG. 1B). In addition, expression of TGFβRII was reduced to nearly nil on HC-HA/PTX3 after TGFβ1 challenge, probably causing inhibition of nuclear pSMAD2/3 and cytoplasmic expression of α-SMA (FIG. 1C). Collectively, these results indicated that HC-HA/PTX3 downregulated canonical TGFβ signaling and prevented myofibroblast differentiation that are normally triggered by exogenous TGFβ1. The results supported the notion that: (a) expression of TGFβ and TGFβRII transcripts were downregulated in HCF and human limbal and conjunctival fibroblasts cultured on the AM stromal surface, (b) intrastromal implantation of human AM would not elicit myofibroblast transformation (marked by α-SMA expression) in rabbit corneas in vivo, and (c) soluble AM extracts induce cell aggregation and prevent expression of α-SMA by myofibroblasts, human, and mouse keratocytes seeded on AM stroma maintained their normal phenotype without eliciting nuclear translocation of pSMAD2/3 even if exposed to serum or TGFβ1. Surprisingly, JNK1 overexpression and nuclear translocation were promoted by HC-HA/PTX3+TGFβ1, suggesting that non-canonical TGFβ, that is, JNK1 signaling, is activated.

JNK1 is a repressor of TGFβ1 gene expression as c-Jun NH2-terminal kinase (JNK) has been implicated in the function of transforming growth factor β (TGF-β). This mechanism of regulation of TGFβ signaling by JNK1 represents an unexpected form of cross-talk between two important signaling pathways. Such a crosstalk and inhibition of canonical TGFβ signaling may be important for certain biological functions, for example, reprogramming. In addition, JNK is an upstream regulator of Cyclin D1. c-JUN-N-terminal Kinase can drive Cyclin D1 expression during liver regeneration. In human embryo lung fibroblast model, JNK1 upregulates Cyclin D1. In human lung fibroblasts, silica-induced rapid cycling is mediated through JNK/AP1/Cyclin D1-CDK4-dependent pathway. In such a model, the authors showed dominant negative JNK could reduce percentage of cells in G1-phase. Importantly, cyclin D1 promoter activity is directly controlled by c-Jun. Because it has been shown that canonical TGFβ signaling in HCF was inhibited by HC-HA/PTX3+TGFβ1 through downregulation of TGFβRII by Cyclin D1 and these preliminary data also showed that transcription of JNK1 is activated only by HC-HA/PTX3+TGFβ1, but not by either HC-HA/PTX3 or TGFβ1 alone, thus it was speculated that HC-HA/PTX3+TGFβ1 but not HC-HA/PTX3 alone activates JNK-cJUN-Cyclin D signaling, which is responsible for suppressing of canonical TGF-b signaling.

Interestingly, HC-HA/PTX3 promoted overexpression of NC markers such as p75NTR, and overexpression of other NC markers such as HNK1, KLF4, Snail1 in the absence of TGFβ1 (FIG. 2A), collectively indicating that HCF seeded on HC-HA/PTX3 have been reversed to younger stromal cells in the absence of TGFβ1. In contrast, in the presence of TGFβ1, unique mRNA upregulation and nuclear translocation of p75NTR, with significant overexpression of NC markers such as HNK1, Sox9, KLF4, Snail1 and MSX1 in HCF seeded on HC-HA/PTX3 were noted (FIG. 2A). These results suggested that HCF were reprogrammed to neural crest progenitors by HC-HA/PTX3 in the presence of TGFβ1.

To substantiate that HCF were indeed reprogrammed into neural crest progenitors, resultant cells were seeded on the plastic with or without immobilized HA or HC-HA/PTX3 with TGFβ1 for 48 hours, cultured on plastic coated with collagen IV at the density of 20,000 cells/24-well in DMEM+10% FBS for 72 hours before being switched to low glucose DMEM+10% FBS for 3 weeks. In contrast, the spindle cells on HC-HA/PTX3 turned into hexagonal monolayers after 3 weeks of culturing (labeled as iHCEC, i, induced) and expressed similar markers normally found in native HCEC, e.g., Na—K-ATPase and ZO-1 (FIG. 3A). In addition, these results showed that compared to expression of native HCEC, the mRNA expression of endothelial markers Na—K-ATPase, CA2, COL4A4, PITX2, SLC4A4, LEF1, N-cadherin, ZO-1 was all significantly lower in HCF but the expression of COL4A4, α-catenin, β-catenin, and p120 was similar in HCF, while the expression of Na—K-ATPase, CA2, SLC4A4, N-cadherin was lower but expression of PITX2, p120 SLC4A4, LEF1, N-cadherin, ZO-1 was similar, and expression of COL4A4, α-catenin, β-catenin, LEF1 was higher in neural crest like cells (FIG. 3A). In addition, induced HCEC expressed a similar level of CA2, COL4A4, PITX2, α-catenin, β-catenin, LEF1, p120 and ZO-1, but lower level of N-cadherin and higher level of Na—K-ATPase, SLC4A4 when compared to those from native HCEC (FIG. 3A). Furthermore, compared to native HCF fibroblastic markers, the expression of vimentin and CD34 was significantly increased in both HCF and NC cells but not in induced HCEC (FIG. 3B), indicating that those induced HCEC behave like endothelial cells, not like fibroblasts.

As shown, these results have also showed that expression of TGFβRII protein was reduced to nearly nil on HC-HA/PTX3 after TGFβ1 challenge, along with overexpression of mRNA, protein and nuclear translocation of Cyclin D1, which could be attenuated by cyclin D1 siRNA (FIGS. 4A-4F), suggesting that cyclin D plays a significant role in downregulating TGFβRII protein. Such a notion was supported by that downregulation of Cyclin D1 by Cyclin D1 siRNA was accompanied by increase of TGFβRII mRNA, protein and nuclear pSMAD2/3 by HC-HA/PTX3+TGFβ1 (FIGS. 4A-4F). In addition, Cyclin D1 siRNA can also reverse inhibition of α-SMA formation by HC-HA/PTX3 (FIG. 4C. Furthermore, nuclear translocation of p75NTR, overexpression of p75NTR mRNA and protein and overexpression of other NC markers induced by HC-HA/PTX3+TGFβ1 were all inhibited by Cyclin D1 siRNA (FIG. 4G), suggesting that cyclin D played a key role in reprogramming HCF into their progenitor status by HC-HA/PTX3+TGFβ1.

These results showed that only HC-HA/PTX3+TGFβ1 promoted nuclear translocation of CD44-ICD as early as 5 minutes (FIG. 5A), suggesting that HC-HA/PTX3+TGFβ1 activated CD44-ICD signaling through its nuclear translocation. For such an activation, both HC-HA/PTX3 and TGFβ1 were required, and this activation was an immediate and early response. In addition, HC-HA/PTX3+TGFβ1 promoted nuclear translocation of TAK1 at 10 minutes and JNK1 at 15 min (FIG. 5A), suggesting that HC-HA/PTX3+TGFβ1 activated TAK1-JNK1 signaling. This activation was an early response, but lagging CD44-ICD, suggesting that HC-HA/PTX3+TGFβ1 sequentially activated CD44-ICD and TAK1, JNK1, such an activation required both HC-HA/PTX3 and TGFβ1. Furthermore, these results showed that though HA+TGFβ1 and HC-HA/PTX3 alone moderately increased nuclear translocation of Cyclin D1, however, only HC-HA/PTX3+TGFβ1 significantly promoted nuclear translocation of Cyclin D1 as early as 30 min (FIG. 5), suggesting that HC-HA/PTX3+TGFβ1 promotion of nuclear translocation of Cyclin D1 is a much later event, behind CD44-ICD, TAK1 and JNK1. The results indicated that Cyclin D1 may be a downstream target of CD44-ICD and/or TAK1 and/or JNK1. Such an activation required both HC-HA/PTX3 and TGFβ1. Finally, the results showed that HC-HA/PTX3+TGFβ1 promoted nuclear translocation of p75NTR at 45 min, suggesting that HC-HA/PTX3+TGFβ1 promotion of nuclear translocation of p75NTR is a much later event, behind CD44-ICD, TAK1, JNK1 and cyclin D1. The results suggested that 75NTR may be a downstream target of CD44-ICD or TAK1 or JNK1 or Cyclin D1 or their combinations. Such an activation required both HC-HA/PTX3 and TGFβ1.

Marimastat, a broad spectrum MMP inhibitor, or DAPT, a specific GSI [γ-secretase inhibitor (GSI)], or both inhibited nuclear translocation of CD44-ICD at 5 minutes, suggesting that sequential cleavage by MT1-MMP and γ-secretase may be involved in generating CD44-ICD and that Marimastat and DAPT can be used to inhibit nuclear translocation of CD44-ICD at an early event (FIG. 5C). Following this effect by Marimastat or DAPT or both, nuclear translocation of JNK1, Cyclin D1 and p75NTR was also inhibited at later time points. The question is whether the subsequent effect is due to Marimastat and/or DAPT independently or due to suppression of nuclear translocation of CD44-ICD.

Consistent with immunostaining results, the Western blotting results indicated that HC-HA/PTX3+TGFβ1 promoted nuclear translocation of CD44-ICD at 5 min (FIG. 5C). Marimastat, a broad spectrum MMP inhibitor, or DAPT, a specific GSI [γ-secretase inhibitor (GSI)], or both inhibited nuclear translocation of CD44-ICD at 5 minutes (FIG. 5C), suggesting that sequential cleavage by MT1-MMP and γ-secretase was involved in generating CD44-ICD and that Marimastat and DAPT can be used to inhibit nuclear translocation of CD44-ICD at an early event. As to why the cytoplasmic CD44-ICD was still present after inhibition by Marimastat or DAPT or both, it is reasonable to deduce that most of residue cytoplasmic CD44-ICD was still present because CD44-ICD protein half-life is 8 hours. If the inhibitors are pretreated for long time, the cytoplasmic CD44-ICD will disappear so that one would not be able to see whether CD44-ICD nuclear translocation is inhibited by the inhibitors due to no cytoplasmic CD44-ICD. As the results show, HC-HA/PTX3+TGFβ1 activated both MT1-MMP and γ-secretase (FIGS. 6A-6B), which are inhibited by their respective inhibitors. These results suggested that TGFβ1 promoted external cleavage of CD44 via MT1-MMP to permit internal cleavage by γ-secretase to promote nuclear translocation of CD44 ICD in 5 min.

In summary, in the presence of TGFβ1, HC-HA/PTX3 promoted reprogramming of HCF into neural crest progenitors through inhibition of canonical TGFβ signaling, activation of CD44-ICD-TAK1-JNK1-Cyclin D signaling. Such induced neural crest progenitors have multilineages, for example, to differentiate into corneal endothelial-like cells. These results highlight the uniqueness of HC-HA/PTX3 for reprogramming of HCF and alike, for the prospective of HC-HA/PTX3 in clinical applications.

### Example 2: Reversal of Human Corneal Fibroblasts into Keratocytes by HC-HA/PTX3

### Materials and Methods

### Materials

Dulbecco's modified Eagle's medium (DMEM), HEPES buffer, Hanks' balanced salt solution (HBSS), phosphate-buffered saline (PBS), gentamicin, fetal bovine serum (FBS) and Alexa-Fluor-conjugated secondary IgG were purchased from Thermo-Fisher Scientific (Carlsbad, Calif.). Insulin-transferrin-sodium selenite media supplement (ITS) was obtained from Roche Applied Science (Indianapolis, Ind.). Paraformaldehyde, methanol, Triton X-100, Hoechst 33342 dye, SB431542, AMD3100 and monoclonal antibody against β-actin were purchased from Sigma-Aldrich (St Louis, Mo.). Monoclonal antibody against CXCR4 and polyclonal antibodies against keratocan, SDF1, pSMAD2/3, pSMAD1/5 and α-SMA were obtained from Abcam (La Jolla, Calif.). RNeasy Mini Kit was purchased from Qiagen (Valencia, Calif.).

### To Isolate and Culture HCF

A total of 86 human corneas from individuals aged 18-76 years and maintained at 4° C. in Optisol (Chiron Vision, Irvine, Calif.) for less than 7 days after death were obtained from the Florida Lions Eye Bank (Miami, Fla.) and handled according to the declaration of Helsinki. HCF were isolated and cultured. Briefly, the sheets of the epithelium and the endothelium were removed from corneas, the stroma sections were cut into cubes of approximately 1 mm3, incubated in 2 mg/ml collagenase for 16 h at 37° C., and then placed in a culture medium consisting of DMEM supplemented with 10% fetal bovine serum containing 50 mg/ml gentamicin and 1.25 mg/ml amphotericin B. The culture medium was refreshed twice a week. The cells cultured to passage 3 were used for all experiments.

To Induce HCF into the Myofibroblasts

HCF were cultured in DMEM+10% FBS until 70% confluence. The cells were serum-starved for 1 day and treated with 10 ng/ml TGFβ1 for 3 days to induce myofibroblasts.

### To Reverse Human Corneal Fibroblasts or Myofibroblasts to the Keratocytes on Immobilized HC-HA/PTX3

To reverse fibroblasts or myofibroblasts into keratocytes, fibroblasts or myofibroblasts were cultured on immobilized HC-HA/PTX3 in DMEM+10% FBS for 3 days using the samples from the cells on plastic and HA as the controls. Some of the cell cultures were extended to up to 7 days of culture to monitor the expression of α-SMA formation.

### RNA Extraction, Reverse Transcription and Real-Time PCR

Total RNAs were extracted using RNeasy Mini Kit (Qiagen) and reverse-transcribed using High Capacity Reverse Transcription Kit (Applied Biosystems). cDNA was amplified by real-time RT-PCR using specific primer-probe mixtures and DNA polymerase in Quant Studio 5 Real-time PCR System (Applied Biosystems). Real-time RT-PCR profile consisted of 10 min of initial activation at 95° C., followed by 40 cycles of 15 s denaturation at 95° C., and 1 min annealing and extension at 60° C. The genuine identity of each PCR product was confirmed by the size determination using 2% agarose gels followed by ethidium bromide staining together with PCR marker according to EC3 Imaging System (BioImaging System, Upland, Calif.).

### Immunostaining

The samples of human corneal fibroblasts, myofibroblasts and corneal keratocytes were air-dried and fixed in 4% formaldehyde, pH 7.0, for 15 min at room temperature, rehydrated in PBS, incubated with 0.2% Triton X-100 for 15 min, and rinsed 3 times with PBS for 5 min each. After incubation with 2% BSA to block non-specific staining for 30 min, the samples were incubated with the desired first antibody (all at 1:50 dilution) for 16 h at 4° C. After three washes with PBS, they were incubated with corresponding Alexa-Fluor-conjugated secondary IgG (all 1:100 dilution) for 60 min. The samples were then counterstained with Hoechst 33342 and analyzed in Zeiss LSM 700 confocal microscope (Thornhood, N.Y.). Corresponding mouse and rabbit sera were used as negative controls for primary monoclonal and polyclonal antibodies, respectively.

### Western Blotting

Cell lysates were prepared in RIPA buffer and resolved on 4-15% (w/v) gradient acrylamide gels under denaturing and reducing conditions for Western blotting. The protein extracts were transferred to the nitrocellulose membrane, which was then blocked with 5% (w/v) fat-free milk in TBST [50 mM Tris-HCl, pH 7.5, 150 mM NaCl, 0.05% (v/v) Tween-20], followed by sequential incubation with the specific primary antibody against keratocan and its respective horseradish peroxidase (HRP)-conjugated secondary antibody using β-actin as the loading control. Immunoreactive proteins were detected with Western Lighting Chemiluminescence (PerkinElmer, Waltham, Mass.).

### Timing Course Analysis of SDF1/CXCR4 and BMP Signaling

HCF were seeded on plastic in DMEM+10% FBS and treated with/without PBS or HA or HC-HA/PTX3 with or without CXCR4 inhibitor AMD3100 or BMP inhibitor SB431542 for 0, 5, 15, 30, 45, 60 min, 24 and 48 h before being harvested for real-time PCR of SDF1, CXCR4 and BMPs, for immunostaining of CXCR4 and pSMAD1/5.

### Statistical Analysis

All summary data were reported as means±s.d. calculated for each group and compared using ANOVA and the Student's unpaired t-test by Microsoft Excel (Microsoft, Redmont, Wash.). Test results were reported as two-tailed P values, where p<0.05 was considered statistically significant.

### Results

Human Corneal Myofibroblasts could Form Aggregates, be Reversed to Keratocytes by HC-HA/PTX3

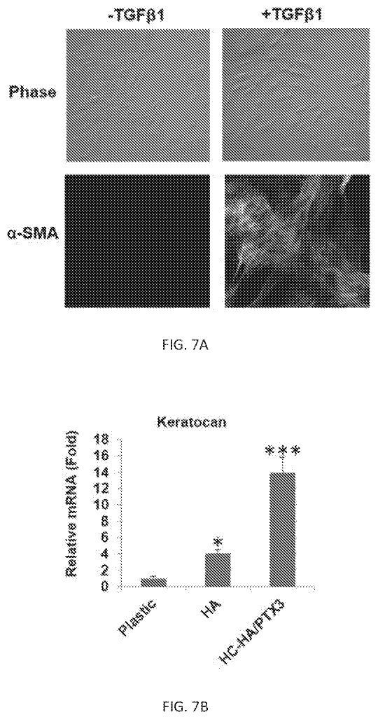

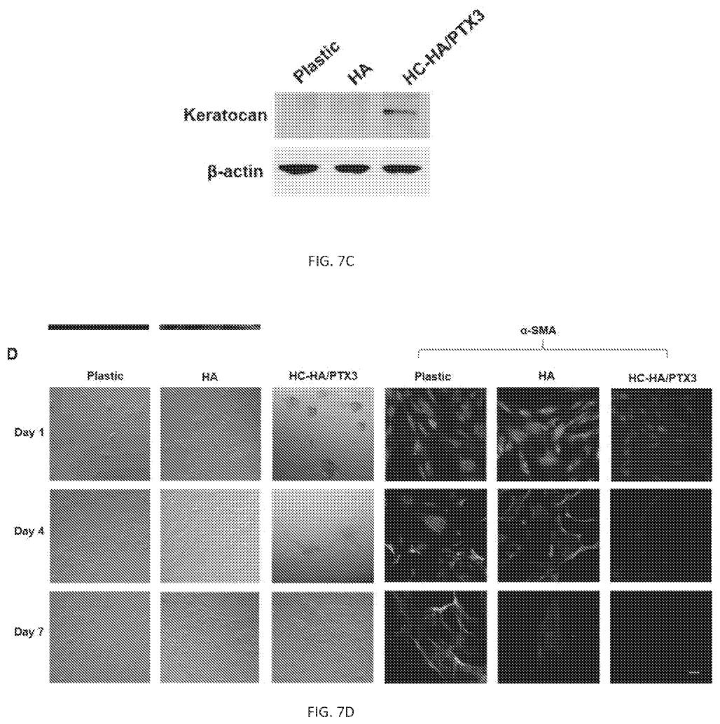

Previously, it has been demonstrated that AM stromal extract can reverse the myofibroblasts originated from AM stromal cells to the fibroblasts. It is unclear whether HC-HA/PTX3 complex extracted from AM can further reverse myofibroblasts to even younger keratocyte-like progenitors. To answer this question, myofibroblasts were first induced from HCF. Specifically, HCF were treated with 10 ng/ml TGFβ1 for 3 days in DMEM+ITS to induce the myofibroblasts. The induced myofibroblasts were verified by immunostaining of α-SMA (FIG. 7A). To determine whether HC-HA/PTX3 complex might reprogram myofibroblasts, the reprogrammed cells on HC-HA/PTX3 complex were monitored for up to 7 days. The myofibroblasts formed aggregates on HC-HA/PTX3 but not on plastic or HA at day 1 (FIG. 7D), Immunostaining results showed that the staining of α-SMA on HC-HA/PTX3 but not plastic and HA was significantly reduced at day 1 and completely disappeared at day 4 and day 7 (FIG. 7D). Interestingly, HC-HA/PTX3 induced a 14-fold increase of keratocan mRNA expression, along with similar increase of keratocan protein expression (FIG. 7B-7C).

HCF could Also Form Aggregates, be Reversed to Keratocytes and Resistant to TGFβ1 on HC-HA/PTX3

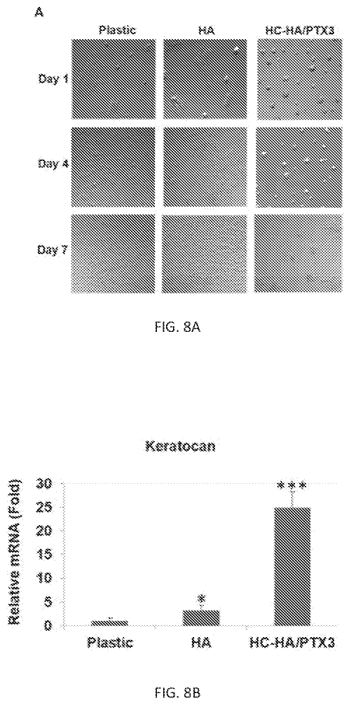

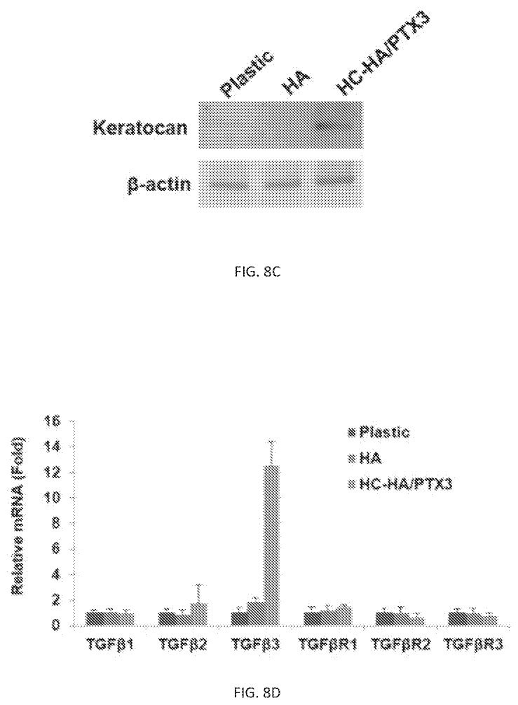

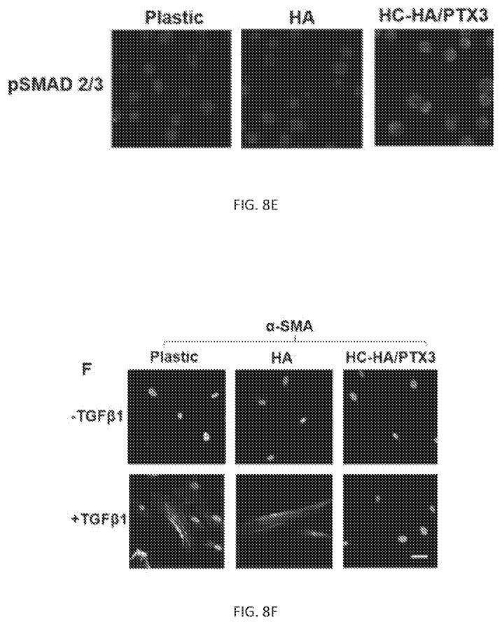

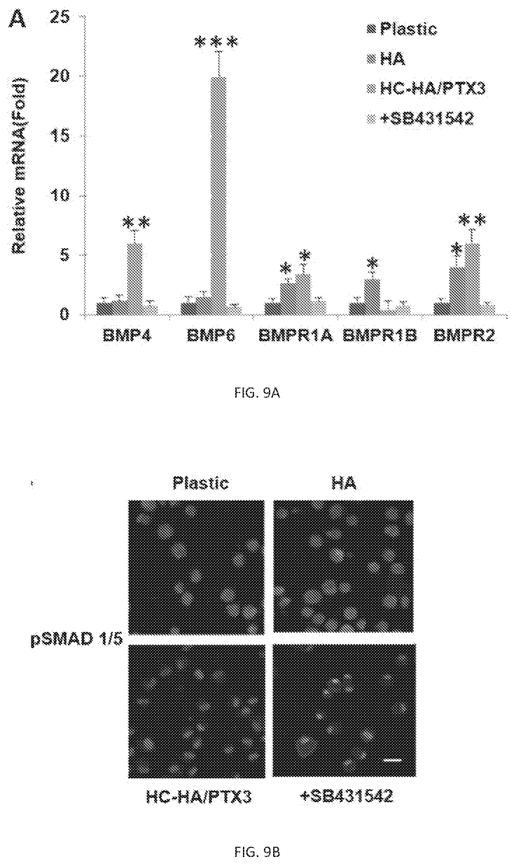

To determine whether HC-HA/PTX3 might do the same or better to reverse HCF to younger keratocytes, HCF were monitored on HC-HA/PTX3 for up to 7 days. As shown, the fibroblasts formed some aggregates on plastic or HA, most of which were spindle, in contrast to those in aggregates on HC-HA/PTX3 at day 1 (FIG. 8A). In addition, the aggregation was continued on HC-HA/PTX3 but not plastic or HA for up to 7 days (FIG. 8A). Furthermore, TGFβ signaling was not activated even under challenge of TGFβ1 (no nuclear staining of pSMAD2/3 and no α-SMA staining in HCF on HC-HA/PTX3, not on plastic or HA, FIG. 8D, FIG. 8E and FIG. 8F except 12-fold increase of TGFβ3, an anti-TGFβ format by HC-HA/PTX3 only). As a result, transcript expression of keratocan was elevated by 24-fold, with similar increase of keratocan proteins (FIG. 8B and FIG. 2C).

### Reversal of HCF to Keratocytes Mediated by Canonical BMP Signaling

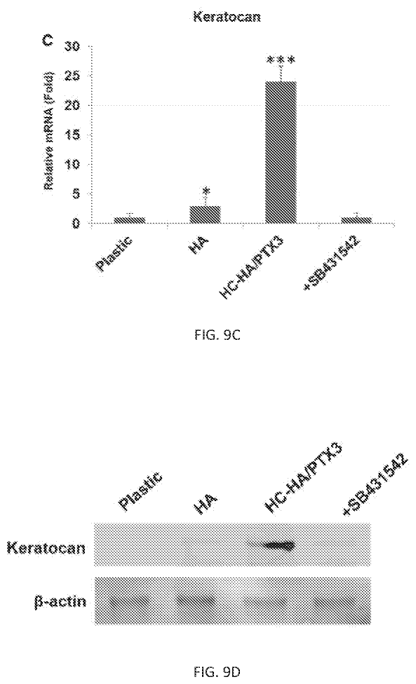

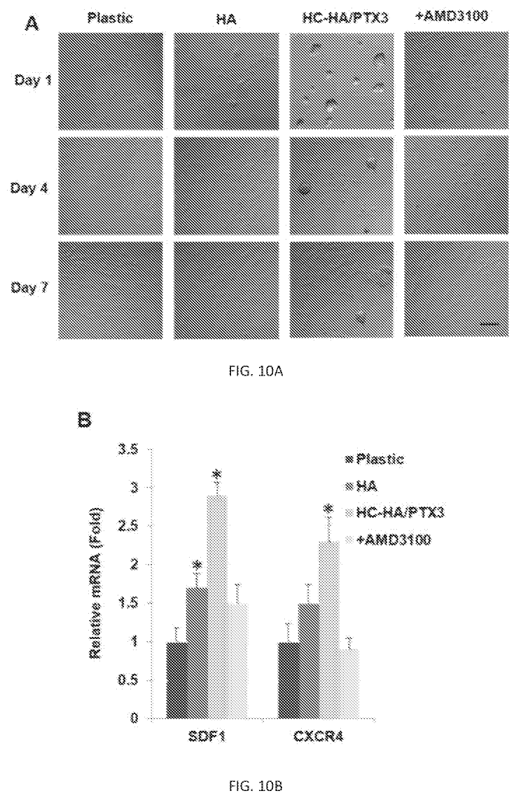

To determine which signaling(s) was involved in reversal of HCF to keratocytes by HC-HA/PTX3, the fibroblasts were seeded directly on immobilized HC-HA/PTX3 with or without BMP inhibitor SB431542 in MESCM for 24 h for determination of mRNA and for 48 h for quantitation of protein using plastic and HA as the controls. As expected, HC-HA/PTX3 induced 6- and 20-fold mRNA increase of BMP4 and BMP6 and 3- and 5-fold transcript increase of BMPR1A and BMPR2 respectively (FIG. 9A). In addition, HC-HA/PTX3 promoted pSMAD1/5 nuclear translocation (FIG. 9B). As a result, HC-HA/PTX3 stimulated 23-fold increase of keratocan mRNA and similar increase of keratocan protein (FIG. 9C and FIG. 9D). The conclusion that reversal to keratocytes by activation of canonical BMP signaling was confirmed by use of BMP inhibitor SB4315412, which completely inhibited BMP signaling, and thus, keratocan expression in mRNA and protein levels.

### Aggregation Mediated by SDF1-CXCR4 Signaling Regulated Canonical BMP Signaling-Mediated Reversal to Keratocytes

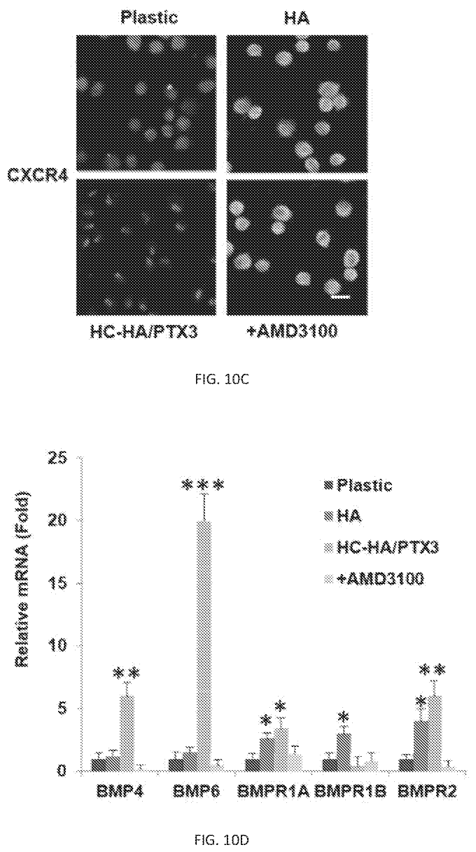

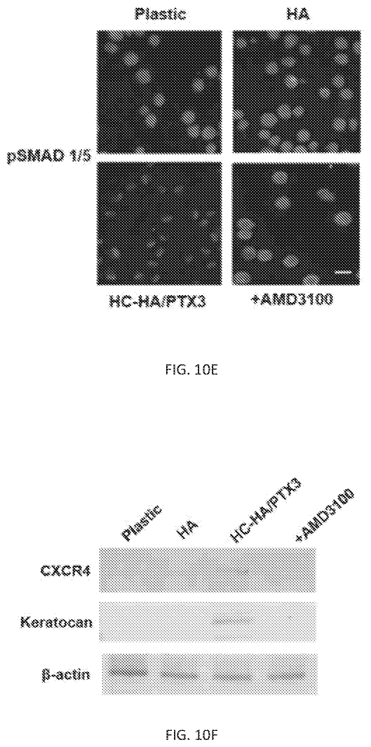

To determine whether aggregation was mediated by SDF1-CXCR4 signaling and if so, whether such signaling mediated canonical BMP signaling-regulated reversal to keratocytes, the CXCR4 inhibitor, AMD3100, was used to block SDF1-CXCR4 signaling. Indeed, SDF1-CXCR4 signaling was activated by HC-HA/PTX3, evidently by overexpression of 3-fold of increase of SDF1 transcript and 2-fold of increase of CXCR4 mRNA (FIG. 10B) and nuclear translocation of CXCR4 (FIG. 10C). Blockade of SDF1-CXCR4 signaling by AMD3100, completely attenuated cell aggregation (FIG. 10A), mRNA overexpression of SDF1 and CXCR4 (FIG. 10B) and CXCR4 nuclear translocation (FIG. 10C). As a result, the transcript expression of BMP4, BMP6, BMPR1A, BMPR1B and BMPR2 (FIG. 10D and FIG. 10E) and nuclear translocation of pSMAD1/5 (FIG. 10E) were all completely nullified by AMD3100 in addition of blockade of CXCR4 nuclear translocation and of keratocan protein expression (FIG. 10F).

### Activation of SDF1/CXCR4 was Followed by BMP Signaling

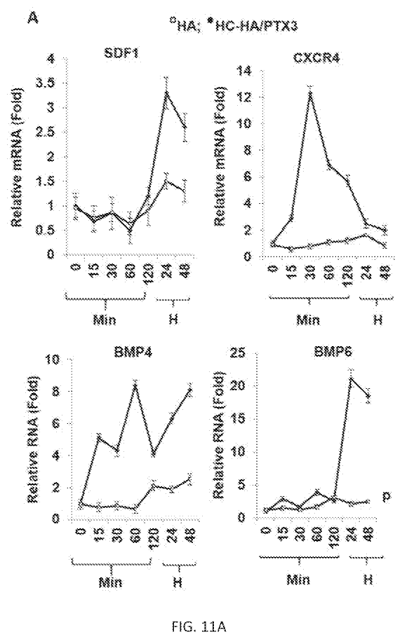

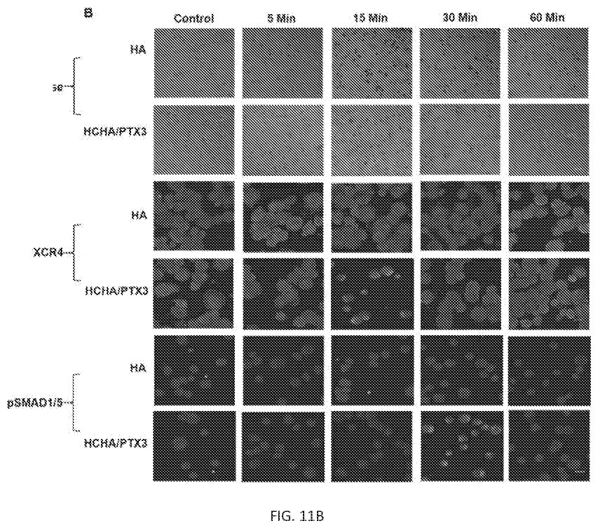

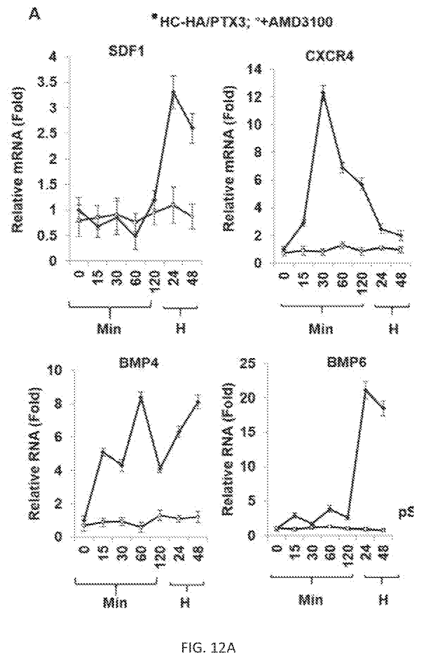

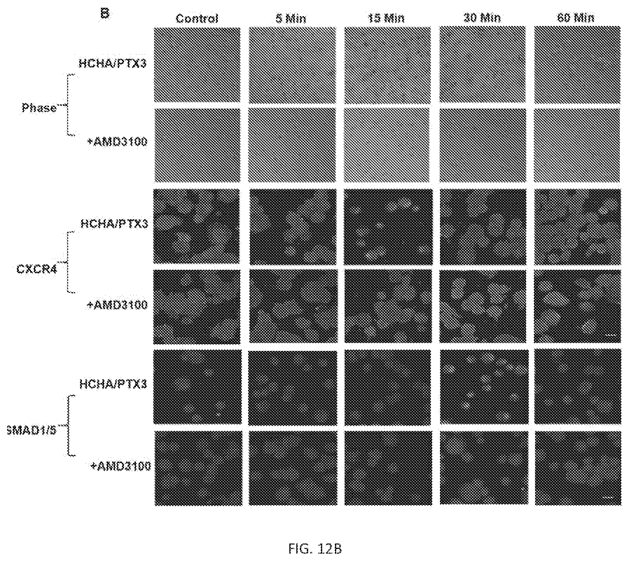

Previously, it was reported cell-cell reunion between LNC and SC is mediated by CXCR4/SDF-1 axis, in which CXCR4 is strongly expressed by limbal stromal NCs and SDF-1 is expressed by limbal epithelial progenitors. It remains unclear whether cell aggregation in HCF is mediated by CXCR4/SDF-1 axis. In addition, HC-HA/PTX3 has been shown to uniquely promote BMP signaling in early P4 LNC. It is also unknown whether HC-HA/PTX3 promotes the sustained activation of BMP signaling in HCF, and if so whether BMP signaling is medicated by SDF1-CXCR4 signaling or vice versa in HCF. Therefore, a time-course was performed to determine the time line of SDF1-CXCR4 and BMP signaling. The results showed that HC-HA/PTX3, not HA, promoted aggregation in HCF in 60 min (FIGS. 11A-11B). In addition, HC-HA/PTX3, not HA, promoted CXCR4 mRNA expression at 15 minutes, which peaked at 30 minutes, with its nuclear translocation at 15 min (FIGS. 11A-11B). In contrast, the expression of SDF1 was not significantly promoted by HC-HA/PTX3 until 24 h. In addition, HC-HA/PTX3 promoted zig-zag overexpression of BMP4 (15 min to 48 h) but late overexpression of BMP6 (24 h and 48 h) with pSMAD1/5 nuclear translocation at 30 minutes in HCF (FIGS. 11A-11B). The results suggested that SDF1-CXCR4 signaling was ahead of BMP signaling.

### SDF1/CXCR4 Signaling Mediated Aggregation and BMP Signaling

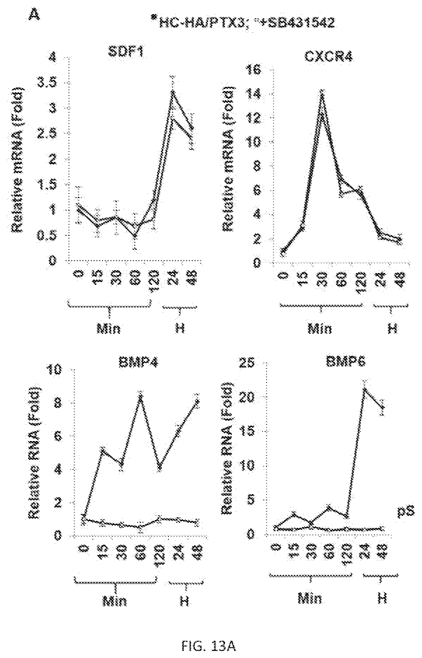

Previously, inhibition of CXCR4 by AMD3100 or a blocking antibody to CXCR4 has been shown at the time of seeding to disrupt their reunion, resulting in epithelial spheres that exhibit more corneal differentiation and a notable loss of holoclones. In addition, it has been demonstrated that HC-HA/PTX3 activated BMP signaling in P4 LNC. It remains unclear whether HC-HA/PTX3 promotes activation of BMP signaling or vice visa. To determine the relationship between SDF1/CXCR4 signaling, specific BMP inhibitor AMD3100 was used. The result showed that inhibition of SDF1/CXCR4 signaling by AMD3100 completely blocked aggregation, not only expression of SDF1 and CXCR4, but also that of BMP4 and BMP6 induced by HC-HA/PTX3 in HCF (FIG. 12A-12B). In addition, nuclear translocation of CXCR4 and pSMAD1/5 was also eliminated (FIG. 12A-12B). The results indicated that SDF1/CXCR4 signaling mediated BMP signaling.

### BMP Signaling Did not Affect SDF1-CXCR4 Signaling and Aggregation

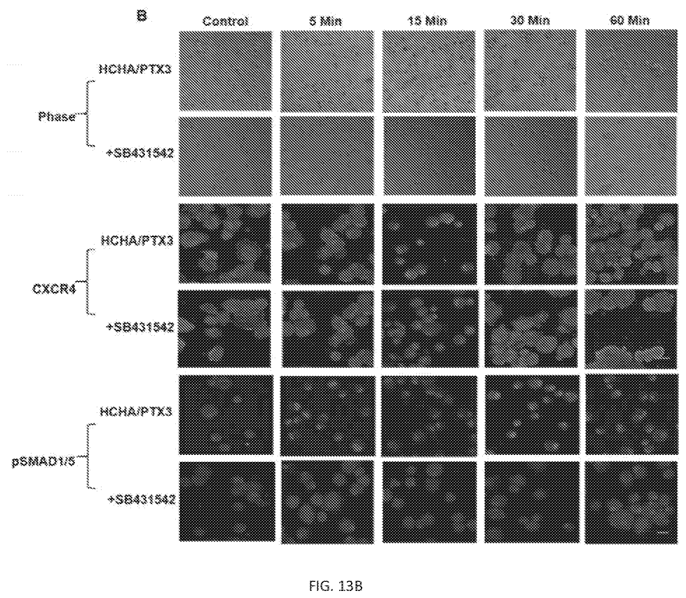

To confirm the view that SDF1/CXCR4 mediated aggregation and BMP signaling, not vice versa, BMP inhibitor SB431542 was used. As expected, the results demonstrated that inhibition of BMP signaling by SB431542 did not affect aggregation, expression of SDF1 and CXCR4 and nuclear translocation of CXCR4, but completely inhibited expression of BMP4 and BMP6 and nuclear translocation of pSMAD1/5 induced by HC-HA/PTX3 in HCF (FIGS. 13A-13B). The results confirmed that SDF1/CXCR4 signaling mediated BMP signaling, not vice versa.

### Discussion

Since first reintroduced two decades ago, amniotic membrane (AM) transplantation has become a standard surgical procedure for ocular surface reconstruction to deliver anti-inflammatory, anti-angiogenic, and anti-scarring actions and to promote wound healing. From soluble AM extracts, HC-HA/PTX3 has been purified and characterized as a unique matrix component responsible for the aforementioned AM's therapeutic actions. HC-HA/PTX3 is formed by tight association with pentraxin 3 (PTX3) of HC-HA, which consists of high molecular weight hyaluronic acid (HA) covalently linked to heavy chain 1 (HC1) of inter-α-trypsin inhibitor through the catalytic action of tumor necrosis factor-stimulated gene-6 (TSG-6). Although human and murine keratocytes can maintain their phenotype without differentiating into α-SMA-expressing myofibroblasts, however, it is unclear whether immobilized HC-HA/PTX3 extracted from AM can reverse terminal differentiated human corneal myofibroblasts into keratocytes. To determine whether HC-HA/PTX3 may reverse human corneal myofibroblasts to keratocytes, the reversed cells within 7 days of culture on HC-HA/PTX3 using plastic and HA as the controls were characterized. It was discovered that the induced myofibroblasts may form aggregates on HC-HA/PTX3, but not on plastic or HA, at day 1 and day 4 (FIG. 7A) with cell shape changes from elongated to small and round shape, indicating that those myofibroblasts may be reversed into much younger progenitors. In addition, the myofibroblasts on plastic or HA retained their characteristic staining of α-SMA during the entire culture period, but the cells on HC-HA/PTX3 showed significantly reduced α-SMA at day 1 and negative staining at day 4 and day 7 (FIG. 7D), supporting our notion that the myofibroblasts have been reversed to younger progenitors. Further analysis suggests that such cells are keratocytes, expressing mRNA and protein of keratocan, a specific keratocyte marker (FIGS. 7B-7C).

To determine whether HC-HA/PTX3 may also reverse HCF to younger progenitors, cells were characterized within 7 days of culture on HC-HA/PTX3 using plastic and HA as the controls. Indeed, HCF may form more aggregates than myofibroblasts on HC-HA/PTX3 with cell shape changes from elongated to small and round shape, but not on plastic or HA, until day 7 (FIG. 8A), indicating that those fibroblasts may also be reversed to keratocytes on HC-HA/PTX3. Further analysis demonstrated that the cells on plastic or HA retained their characteristic staining of α-SMA during the entire culture period, however, the cells on HC-HA/PTX3 showed significantly reduced α-SMA at day 1 and negative at day 4 and day 7, reinforce our notion that the fibroblasts have been reversed to younger progenitors. Such reversed cells also express mRNA and protein of keratocan, showing that those cells are indeed keratocytes (FIGS. 8B-8C). Interestingly, TGFβ signaling was completely inhibited (only the anti-TGFβ form, TGFβ3 was enhanced by HC-HA/PTX3, FIG. 8D). The significance of such inhibition requires further investigation.

Previously, it has been reported that in mouse ES cells (mESCs), BMP signaling is important for maintaining the pluripotent state. A systematic siRNA screening further uncovered a key role for BMP signaling and the induction of mesenchymal-to-epithelial transition (MET). Because HC-HA/PTX3 uniquely promotes BMP signaling in early P4 LNC, it was wondered whether BMP signaling plays important regulatory roles in reversal of fibroblasts to keratocytes. Herein, HC-HA/PTX3 have been shown to have significantly promoted mRNA expression of BMP4, BMP6, BMPR1A, BMPR2 (FIG. 9A) and nuclear translocation of pSMAFD1/5 (FIG. 9B), suggesting that BMP signaling is indeed activated. Inhibition of BMP signaling by BMP inhibitor SB431542 completely blocks overexpression of BMP4, BMP6, BMPR1A, BMPR2 and nuclear translocation of pSMAFD1/5, and mRNA and protein expression of keratocan (FIGS. 9C-9D), suggesting that BMP signaling plays a key role in the reversal of fibroblasts to keratocytes.

Previously, it has been reported that single SCs and NCs could reunite to generate sphere growth in three-dimensional Matrigel™ in the embryonic SC medium, and that such sphere growth initiated by SC-NC reunion was mediated by SDF-1 uniquely expressed by limbal epithelial progenitor cells and its receptor CXCR4, but not CXCR7, strongly expressed by limbal stromal NCs. Because the reversal to keratocytes was associated with cellular aggregation, it was wondered whether such aggregation was also mediated by SDF1-CXCR4 signaling. The results indeed showed that aggregation by HC-HA/PTX3 was associated with SDF1-CXCR4 signaling, evidently by increase of mRNA expression of SDF1 and CXCR4 (FIG. 10B), increase of CXCR4 protein expression (FIG. 10F) and CXCR4 nuclear translocation (FIG. 10C). Such an event is associated with activation of BMP signaling, evidently overexpression of BMPs, BMPRs (FIG. 10D) and pSMAD1/5 nuclear translocation (FIG. 10E).

To determine whether the reversal to keratocytes is linked to aggregation, SDF1-CXCR4 and BMP signaling, CXCR4 inhibitor, AMD3100 and BMP inhibitor, SB431542 were used. As expected, AMD3100 completely blocked mRNA overexpression of BMPs and BMPRs, prevented nuclear translocation of pSMAD1/5, inhibited mRNA and protein expression of keratocan (FIGS. 10D-10F), indicating the reversal of HCF to keratocytes are via activation of SDF1-CXCR4-mediated canonical BMP signaling.

To confirm whether the reversal to keratocytes was mediated by SDF1-CXCR4-BMP signaling, a time course within 1 h was conducted in HCF using soluble HC-HA/PTX3 with and without CXCR4 inhibitor AMD3100 or BMP inhibitor SB431542. The results showed that without any inhibitors, HC-HA/PTX3 promoted aggregation in HCF in 60 min, CXCR4 mRNA overexpression at 15 minutes, which peaked at 30 minutes, with CXCR4 nuclear translocation at 15 min (FIGS. 11A-11B). In addition, HC-HA/PTX3 promoted overexpression of BMP4 (15 min to 48 h) and BMP6 (24 h and 48 h) with pSMAD1/5 nuclear translocation at 30 minutes in HCF (FIGS. 11A-11B), indicating that SDF1-CXCR4 signaling is ahead of BMP signaling.

To determine whether activation of SDF1-CXCR4 signaling mediated BMP signaling by HC-HA/PTX3, CXCR4 inhibitor AMD3100 and BMP inhibitor SB431542 were used. The results showed that AMD3100 completely blocked aggregation, SDF1 and CXCR4 expression and nuclear translocation of CXCR4, mRNA overexpression of BMPs and BMPRs, prevented nuclear translocation of pSMAD1/5, inhibited transcript and protein expression of keratocan (FIGS. 12A-12B) while BMP inhibitor SB431542 only blocked transcript overexpression of BMPs and BMPRs and nuclear translocation of pSMAD1/5, and transcript and protein overexpression of keratocan but not overexpression of SDF1 and CXCR4, and nuclear translocation of CXCR4 (FIGS. 12A-12B), indicating the reversal of HCF to keratocytes are via activation of SDF1-CXCR4-canonical BMP signaling.

To summarize, terminal differentiated myofibroblasts were shown to be reverted to younger keratocytes by immobilized HC-HA/PTX3 via activation of SDF1-CXCR4-canonical BMP signaling. Such a method may be applied to generation of younger functional progenitors, and ultimately, regeneration of clinically applicable tissues.

### Example 3: HC-HA/PTX3 Purified from Human Amniotic Membrane Reverts Late Passaged Limbal Niche Cells to Nuclear Pax6+ Neural Crest Progenitors by Promoting Cell Aggregation Via SDF-1/CXCR4 Signaling

### Materials and Methods

### Materials Isolation, Expansion and Treatment of Human Limbal Niche Cells

Human corneolimbal rim and central cornea button stored at 4° C. in Optisol (Chiron Vision, Irvine, Calif.) for less than 7 days were obtained from donors (Florida Lions Eye Bank, Miami, Fla.). Tissue were rinsed three times with PBS pH 7.4 containing 50 μg/mL gentamicin and 1.25 μg/mL amphotericin B, the excess sclera, conjunctiva, iris, corneal endothelium and trabecular meshwork were removed up to the Schwalbe's line for the corneoscleral rim before being cut into superior, nasal, inferior, and temporal quadrants at 1 mm within and beyond the anatomic limbus. An intact epithelial sheet including basal epithelial cells was obtained by subjecting each limbal quadrant to digestion with 10 mg/ml dispase in modified embryonic stem cell medium (MESCM), which was made of Dulbecco's Modified Eagle's Medium (DMEM)/F-12 nutrient mixture (F-12) (1:1) supplemented with 10% knockout serum, 10 ng/mL LIF, 4 ng/mL bFGF, 5 mg/mL insulin, 5 mg/mL transferrin, 5 ng/mL sodium selenite supplement (ITS), 50 μg/mL gentamicin and 1.25 μg/mL amphotericin B in plastic dishes containing at 4° C. for 16 h under humidified 5% CO₂ incubator. LNC were isolated by digestion with 2 mg/mL collagenase A at 37° C. for 16 h to generate floating clusters.

For expansion, single cells derived from limbal clusters after digestion with 0.25% trypsin and 1 mM EDTA (T/E) were seeded at 1×cm⁴/cm² in the 6-well plate pre-coated with 5% Matrigel™ in MESCM and cultured in humidified 5% CO₂ with media change every 3-4 days for total 6-7 days. For in vitro 3D Matrigel™, P10 LNC were seeded in 3D Matrigel™ at the density of 5×10⁴ cell s/cm² to generate aggregates in MESCM for 48 h. For in vitro time course study, cell aggregations were monitored by phase microscope by Zeiss Axio-Observer Z1 Motorized Inverted Microscope (Carl Zeiss, Thornwood, N.Y.).

Upon 80% confluence, P10 LNC cultured on coated Matrigel™ (MG) were pre-treated with 0.1% DMSO with or without 20 μg/mL AMD3100 or 100 nM LDN-193189 for 30 min before being trypsinized and seeded at 2×10⁵/mL on coated MG in MESCM containing 20 μg/mL of AMD3100 or 100 nM LDN-193189 with 25 μg/mL soluble HC-HA/PTX3 for another 48 h. For the siRNA knockdown, 80% confluent P10 LNC on 6-well coated MG were subjected to transfection by mixing 200 μl of serum-free, antibiotic-free MESCM with 4 μL of HiPerFect siRNA transfection reagent (final dilution, 1:300) and 6 μl of 20 μM of scRNA or siRNAs for BMPR1A, BMPR1B, BMPR2, and ACVR1 at the final concentration of 100 nM, drop-wise, followed by culturing in 1 mL of fresh MESCM at 37° C. for 24 h before soluble HC-HA/PTX3 was added at a final concentration of 25 μg/mL in the MESCM medium.

### Purification and Immobilization of HC-HA/PTX3

HC-HA/PTX3 was purified from cryopreserved human placentas provided by Bio-Tissue, Inc. (Miami, Fla.) with modification. In brief, AM retrieved from placenta was cryopulverized by FreezeMill (FreezerMill 6870, SPEX® SamplePrep, Metuchen, N.J.), extracted by PBS (pH 7.4) at 4° C. for 1 h, and the centrifuged at 48,000×g at 4° C. for 30 min to generate the supernatant which was designated as AM extract. This extract was then fractionated by ultracentrifugation in a CsCl gradient at an initial density of 1.35 g/ml in 4 M GnHCl at 125,000 g at 15° C. for 48 h (Optima™ L-80 X, SW41 rotor, Beckman Coulter, Indianapolis, Ind.). A total of 12 fractions (1 ml/fraction) were collected from each ultracentrifuge tube. The weight of each fraction was measured to calculate the density, while HA content and protein content in each fraction were measured by the enzyme-linked immunosorbent HA Quantitation Test Kit (Corgenix, Broomfield Colo.) and the BCA Protein Assay Kit (Life Technologies, Grand Island, N.Y.), respectively. The fractions of 2-12 which contained most of HC-HA/PTX3 were pooled and were further subjected to three consecutive runs of ultracentrifugation at 125,000 g in CsCl/4 M guanidine HCl at a density of 1.40 g/mL for the 2nd run and 1.42 g/mL for 3^{rd} and 4^{th} run, each run at 15° C. for 48 h. The fractions 3-9 after the 4th run containing HC-HA/PTX3 but little other proteins were pooled and dialyzed against distilled water at 4° C. for 48 h with a total of 5 times of water change, lyophilized, and stored at −80° C. and was designed as HC-HA/PTX3. Before use, HC-HA/PTX3 was qualified by verifying its biochemical composition containing high molecular weight HA based on agarose gel electrophoresis.

The presence of HC1 (ab70048, Abcam, Cambridge, Mass.) and PTX3 (ALX-804-464-C100, Enzo Life Sciences, Farmingdale, N.Y.) in purified HC-HA/PTX3 with or without HAase digestion (1 U/μg HA) in the presence of protease inhibitors, Sigma-Aldrich, St. Louis, Mo.) was validated. Because the negligible amount of protein therein, the amount of HC-HA/PTX3 used in the experiment was expressed based on the HA amount.

HC-HA/PTX3 was immobilized on Covalink-NH 96 wells (Pierce) by first sterilizing the Covalink-NH 96 wells in 70% alcohol for 30 min and then the wells were washed with distilled water two times. HC-HA/PTX3 (2 μg/well) with the crosslinking reagents of Sulfo-NHS at 9.2 mg/mL (Pierce) and 1-ethyl-3(3-dimethylaminopropyl) carbodiimide (Pierce) at 6.15 mg/ml were added to each well (100 μl) and incubated at 4° C. overnight. After that, the un-crosslinked HC-HA/PTX3 and crosslinking reagents were removed and the wells were washed twice with 2 M NaCl/50 mM MgSO4/PBS, followed by two washes of PBS.

### Neuroglial Differentiation

A total of 1×10⁴/mL of P10 LNC was seeded on 50 μg/mL poly-L-ornithine and 20 μg/mL laminin-coated or Collagen Type IV coated cover glass in 48-well plate in NSCM supplement with 0.5% N2 and 1% B27 for 2 days. For neuronal differentiation, medium was then replaced to neuronal induction base medium containing DMEM/F12 (1:3) with 0.5% N2 and 1% B27 in additional to 10 ng/mL FGF2 and 20 ng/mL of BDNF (medium A) for 3 days and replaced with base medium in addition to 6.7 ng/mL FGF2 and 30 ng/mL of BDNF for another 3 days. Cell then replaced to base medium in addition to 2.5 ng/mL FGF2, 30 ng/mL BDNF, and 200 mM ascorbic acid for another 8 days. For oligodendrocyte differentiation, medium then replaced with base medium containing DMEM/F12 (1:1) with 1% N2 in addition to 10 ng/mL FGF2, 10 ng/mL PDGF, and 10 μM forskolin for 4 days and then medium was replaced by the base medium in addition to 10 ng/mL FGF2, 30 ng/mL 3,3,5-triiodothyronine, and 200 μM ascorbic acid for another 7 days. For astrocyte differentiation (Thermo Scientific, Santa Clara, Calif.), medium was replaced by DMEM containing 1% FBS, 1% N2, and 2 mM GlutaMax for 10 days. Induction media were changed every 3-4 days.

### Quantitative Real-Time PCR

Total RNAs were extracted from different passaged of LNC by RNeasy Mini Kit (Quiagen, Valencia, Calif.) according to manufacturer's guideline and 1-2 ug of

RNA extract was reverse transcribed to cDNA with reverse-transcribed using High Capacity Reverse Transcription Kit (Applied Biosystems, Foster City, Calif.) using primers listed in Supplementary Table S3. The resultant cDNAs were amplified by specific TaqMan gene expression assay mix and universal PCR master mix in QuantStudio™ 5 Real Time PCR System (ThermoFisher, Santa Clara, Calif.) with real-time RT-PCR profile consisting of 10 min of initial activation at 95° C., followed by 40 cycles of 15 sec denaturation at 95° C., and 1 min annealing and extension at 60° C. The relative gene expression data were analyzed by the comparative CT method (MET). All assays were performed in triplicate. The results were normalized by glyceraldehyde 3-phosphate dehydrogenase (GAPDH) as an internal control.

### Immunofluorescence Staining

Single cells of LNC at different passages were harvested with 0.05% trypsin and 1 mM EDTA at 37° C. for 10 min and prepared for cytospin using Cytofuge (StatSpin Inc., Norwood, Mass.) at 1000 rpm for 8 min. Cells were fixed with 4% formaldehyde, pH 7.0, for 15 min at room temperature, permeabilized with 0.2% Triton X-100 in PBS for 15 min and blocked with 2% bovine serum albumin (BSA) for 1 h before incubated with primary antibodies for 16 h at 4° C. After 3 washes with PBS, the corresponding Alexa Fluor-conjugated secondary IgG (all 1:100 dilution) were incubated for 60 min and 3 washing with PBS. After 3 washes with PBS, the second primary antibodies was incubated for 60 min and followed with the corresponding Alex Fluor-conjugated secondary IgG. The nucleus was counterstained with Hoechst 33342 before being analyzed with Zeiss LSM 700 confocal microscope (Carl Zeiss, Thornwood, N.Y.). Corresponding mouse and rabbit sera were used as negative controls for primary monoclonal and polyclonal antibodies, respectively.

### Statistical Analysis

All summary data were reported as mean±SD. Significance was calculated for each group and compared with two-tailed Student's t-test by Microsoft Excel (Microsoft, Redmond, Wash.). Test results were reported as p values, where p<0.05 were considered statistically significant.

### Results

### Progressive Loss of Nuclear Pax6+ NC Phenotype by Serial Passage of LNC

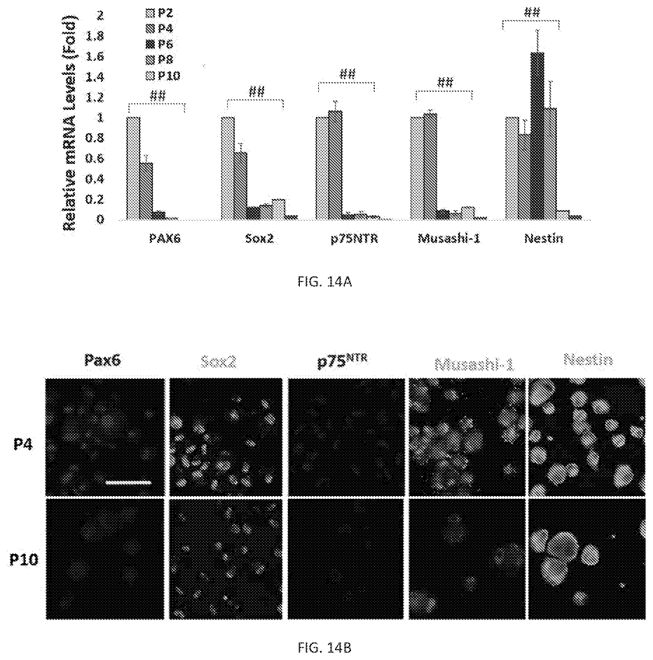

Serial passage of LNC to P10 had been reported to result in the loss of the NC progenitor status has been reported to be characterized by nuclear Pax6 staining, expression of embryonic stem cell (ESC) and neural crest (NC) progenitor markers such as p75NTR, Musashi-1, Sox2, Nestin, Msx1, and FoxD3, and neuroglial differentiation. To confirm this finding, LNC were serially passaged on coated Matrigel™ (MG) in modified embryonic stem cell medium (MESCM) to P10 their phenotypes characterized by transcript expression and immunoassaying. The results indeed confirmed that the transcript expression level of Pax6, Sox2, p75NTR, Musashi-1, and Nestin by P10 LNC was significantly reduced when compared to that of P2 LNC (FIG. 14A, ##p<0.01, n=3 Immunofluorescence staining further confirmed the loss of nuclear staining of Pax6 in P10 LNC, which was accompanied by notable reduction of staining to such NC markers as p75NTR and Musashi-1 when compared to P4 LNC (FIG. 14B).

### Immobilized HC-HA/PTX3 Promotes Cell Aggregation and Reverts P10 LNC to Nuclear Pax6+ NC Progenitors

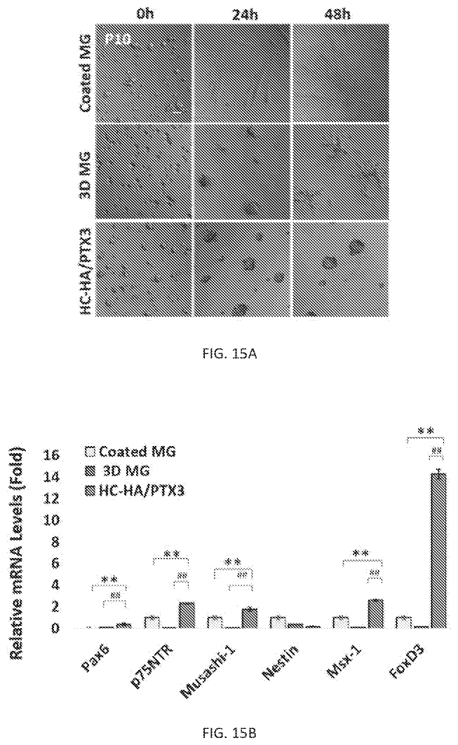

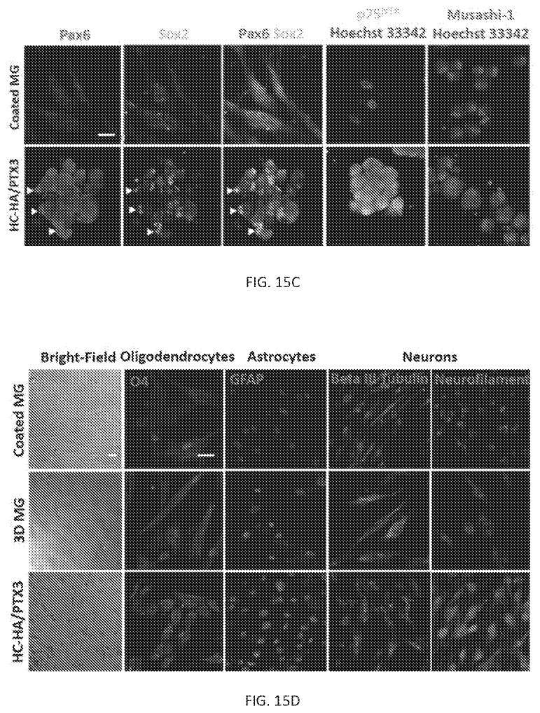

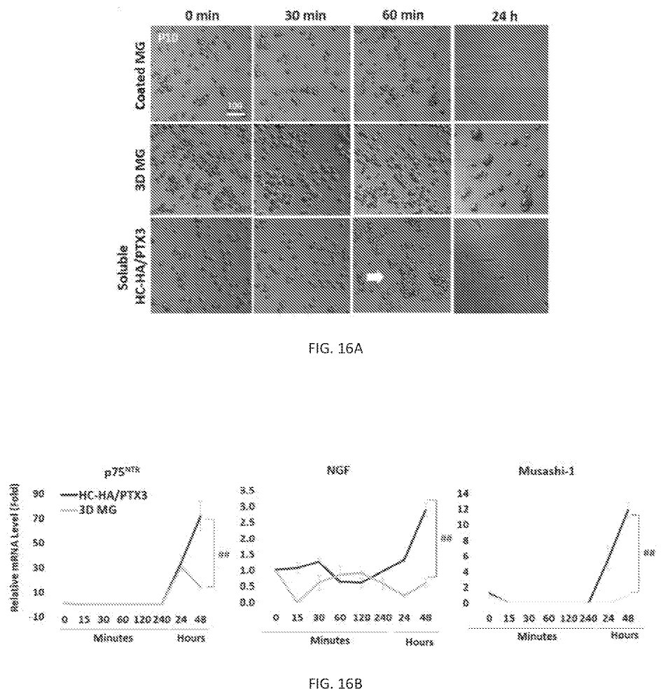

P4 LNC expanded on coated MG in MESCM were found to form cell aggregation when reseeded on 3D MG or immobilized HC-HA/PTX3, of which the latter also helps regain expression of ESC markers. It was thus wondered whether P10 LNC could behave the same to regain the nuclear Pax6+ NC progenitor status by reseeding on immobilized HC-HA/PTX3. Thus, P10 LNC expanded on coated MG were reseeded in MESCM on coated MG, 3D MG or immobilized HC-HA/PTX3 in MESCM for 48 h. Phase contrast microscopy showed that P10 LNC formed cell aggregation in 3D MG and immobilized HC-HA/PTX3 at 24 h and 48 h (FIG. 15A). Quantitative RT-PCR showed that transcript levels of Pax6, p75NTR, Musashi-1, Nestin, Msx-1 and FoxD3 were significantly upregulated in P10 LNC on immobilized HC-HA/PTX3 when compare to on coated MG (FIG. 15B, ** p<0.01, n=3) or 3D MG (FIG. 15B, ##p<0.01, n=3). The immunofluorescence staining confirmed the reappearance of nuclear Pax6 staining and nuclear Sox2 staining (FIG. 15C). The differentiation potential into neurons, oligodendrocytes, and astrocytes by P10 LNC after being re-seeded on 3D MG or immobilized HC-HA/PTX3 was analyzed. Phase contrast microscopy showed that cells exhibited a reduced size and adopted expanded differentiation potential into neurons, astrocytes and oligodendrocytes in P10 LNC when re-seeded on immobilized HC-HA/PTX3 when compared to their counterpart re-seeded in 3D MG (FIG. 15D). Collectively, these results suggested that immobilized HC-HA/PTX3, but not 3D MG, uniquely reverted P10 LNC to nuclear Pax6+ NC progenitors with higher neuroglial differentiation potential.

### Soluble HC-HA/PTX3 Also Promoted Cell Aggregation and Reverted to Pax6+ NC Progenitors

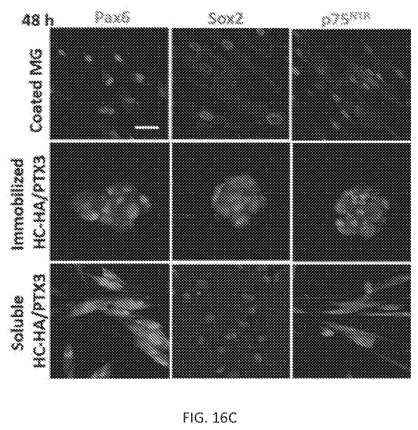

It was then tested whether soluble HC-HA/PTX3 added directly into MESCM in P10 LNC seeded on coated MG might also achieve the same outcome. Phase contrast microscopy showed that cell aggregation was also promoted by soluble HC-HA/PTX3 as early as 60 min (marked by a white arrow) but aggregated cells spread to single spindle cells on coated MG by 24 h while cell aggregation became more prominent in 3D MG (FIG. 16A) similar to what is shown in FIG. 15A. Quantitative RT-PCR revealed significant upregulation of p75NTR and Musashi-1 transcripts by soluble HC-HA/PTX3 at 24 and 48 h when compared to 3D MG (FIG. 16B, ##p<0.01, n=3). Immunofluorescence staining also confirmed nuclear staining of Pax6 and Sox2 and cytoplasmic staining of p75NTR achieved by soluble HC-HA/PTX3 when compared to cells cultured on 3D MG at 48 h (FIG. 16C). Such a staining pattern resembled what was noted on immobilized HC-HA/PTX3 (FIG. 15C).

### Cell Aggregation Promoted by Soluble HC-HA/PTX3 was Mediated by CXCR4/SDF-1 Signaling and Lead to Nuclear Pax6+ NC Progenitors

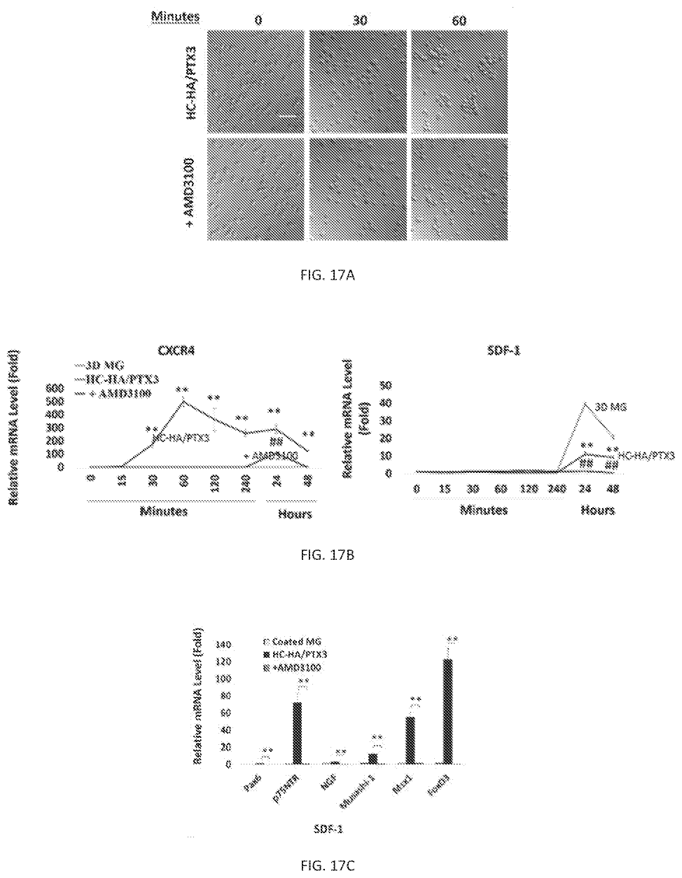

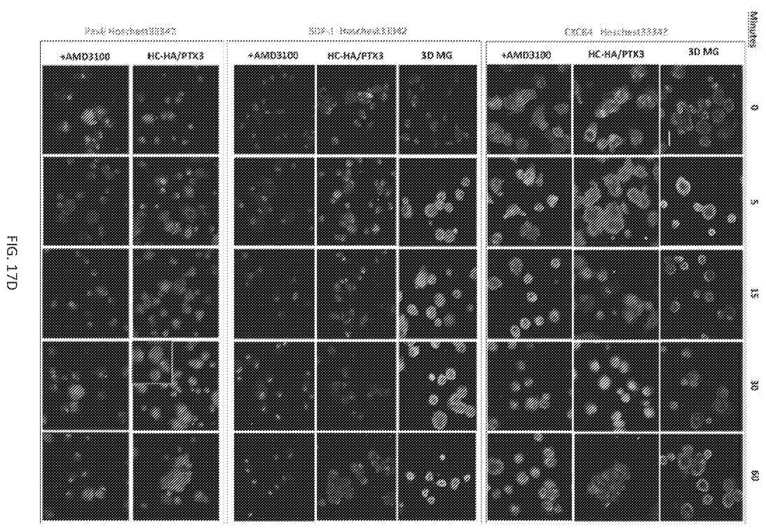

Previously the reunion between P4 LNC and LEPC in 3D MG had been reported to be mediated by CXCR4/SDF-1 signaling with the receptor CXCR4 strongly expressed by LNC and SDF-1 ligand expressed by LEPC and such reunion is pivotal to maintain self-renewal of LEPC. Therefore, it was wondered whether cell aggregation promoted by soluble HC-HA/PTX3 might also be mediated by CXCR4/SDF-1 signaling in P10 LNC. To test this hypothesis, CXCR4/SDF-1 signaling was perturbed by addition of AMD3100, which is a small-molecule CXCR4 inhibitor. Phase contrast microscopy confirmed that cell aggregation was indeed promoted by soluble HC-HA/PTX3 at 60 min in P10 LNC, similar to what was noted above, and that such aggregation was completed aborted by AMD3100 (FIG. 17A). The time course study of the transcript expression by qRT-PCR showed that CXCR4 transcript was marked upregulated by four-fold as early as 15 min and reached a high peak by nearly 500-fold at 60 min when soluble HC-HA/PTX3 was added to P10 LNC on coated MG in comparison to their counterpart in 3D MG (FIG. 17B, ** p<0.01 and ** p<0.01, n=3). Addition of AMD310 significantly downregulated such upregulation of CXCR4 transcript at 24 h and completely aborted at 48 h (FIG. 17B). In contrast, the SDF-1 transcript was not upregulated during the first 60 min in all cultures but was significantly upregulated by 40-fold at 24 h by 3D MG and 10-fold by soluble HC-HA/PTX3, of which the latter was also completely abolished by AMD3100 (FIG. 17B, ##p<0.01, n=3). Immunofluorescence staining of CXCR4 showed membrane/cytoplasmic staining throughout the entire 60 min period on 3D MG; while CXCR4 staining was membrane/cytoplasmic at 0 and 5 min, nuclear at 15 and 30 min, and predominant membranous in cell aggregation at 60 min in soluble HC-HA/PTX3 (FIG. 17D). The latter staining pattern was reverted to that of 3D MG when AMD3100 was added (FIG. 17D). In contrast, the immunostaining of SDF-1 was strongly membranous/cytoplasmic throughout 60 min in cells seeded in 3D MG or soluble HC-HA/PTX3 and became negative after addition of AMD3100 (FIG. 17D). Blockade of CXCR4/SDF-1 signaling by AMD3100 not only prevented cell aggregation promoted by soluble HC-HA/PTX3 but also led to significant downregulation of Pax6, p75NTR, NGF, Musashi-1, Msx-1 and FoxD3 transcripts (FIG. 17C, ** p<0.01, n=3). Furthermore, nuclear Pax6 staining promoted by soluble HC-HA/PTX was aborted by AMD3100 in P10 LNC (FIG. 17D). These data collectively indicated that cell aggregation promoted by soluble HC-HA/PTX3 was mediated by CXCR4/SDF-1 signaling, which was causatively linked to the regain of the nuclear Pax6+ NC progenitor phenotype in P10 LNC.

### CXCR4/SDF-1 Signaling is Required for Activation of BMP Signaling by HC-HA/PTX3

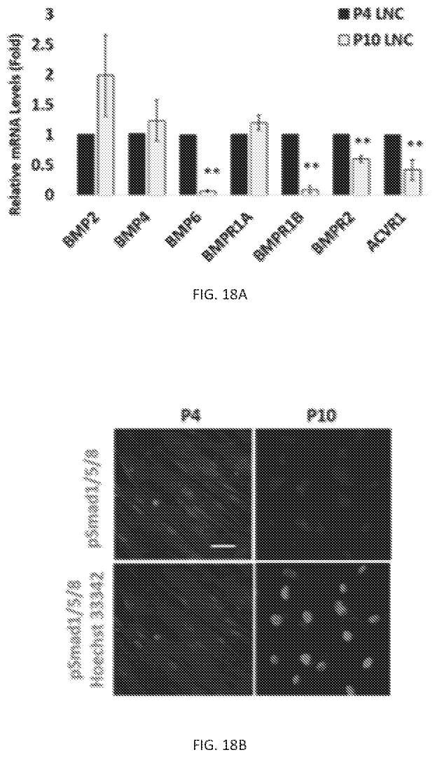

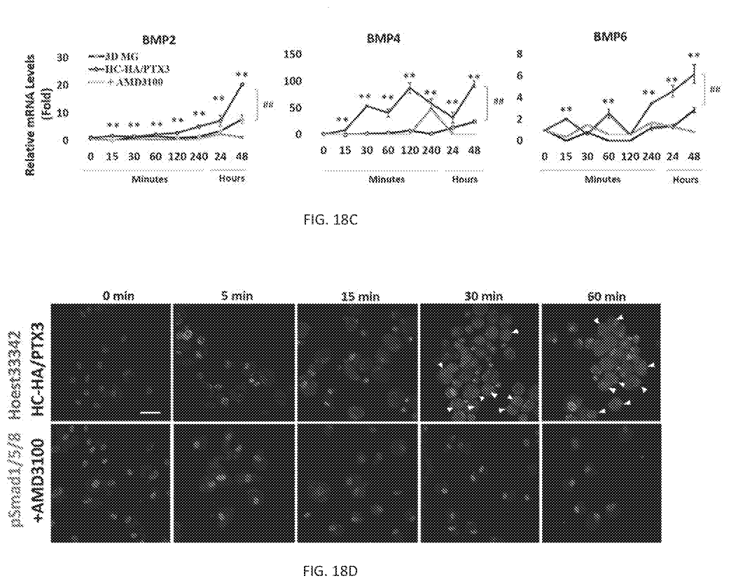

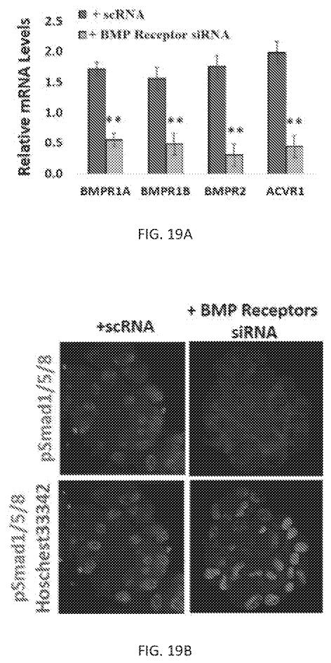

It has been reported that immobilized HC-HA/PTX3, but not 3D MG, uniquely upregulates BMP signaling in P4 LNC, which is responsible for the maintenance of limbal SC quiescence. Thus, it was questioned whether BMP signaling might also be promoted by soluble HC-HA/PTX3 in P10 LNC and if so whether it might be affected by the CXCR4-SDF1 signaling activated by HC-HA/PTX3. qRT-PCR showed that transcript expression of BMP ligands and BMP receptors by P10 LNC was significantly downregulated when compared to P4 LNC expanded on coated MG (FIG. 18A, ** p<0.01, n=3) Immunofluorescence staining confirmed that nuclear localization of pSmad1/5/8 was weakly expressed in P4 LNC and nil in P10 LNC (FIG. 18B). In contrast, qRT-PCR revealed that the expression levels of BMP2, BMP4, BMP6 transcripts were indeed significantly upregulated by soluble HC-HA/PTX3 when compared to 3D MG. (FIG. 18C, ##p<0.01, n=3) Interestingly, the upregulation of BMP4 and BMP6 was as early as 15 min and cyclic to a higher level toward 48 h while that of BMP2 was only noted after 24 h (FIG. 18C). Addition of AMD3100 aborted the transcript levels of BMP2, BMP4, and BMP6 throughout 48 h (FIG. 18C, ** p<0.01, n=3). Immunofluorescence staining further confirmed strong nuclear staining of pSmad1/5/8 indicating canonical BMP signaling was promoted by soluble HC-HA/PTX3 in P10 LNC but was deactivated to cytoplasmic staining after being treated with AMD3100 (FIG. 18D). These findings strongly suggested that CXCR4/SDF-1 signaling promoted by HC-HA/PTX3 was also causally linked to activation of canonical BMP signaling in P10 LNC.

### Suppression of BMP Signaling Did not Affect Nuclear Pax6 Staining and Cell Aggregation Mediated by CXCR4/SDF-1 Signaling Promoted by HC-HA/PTX3

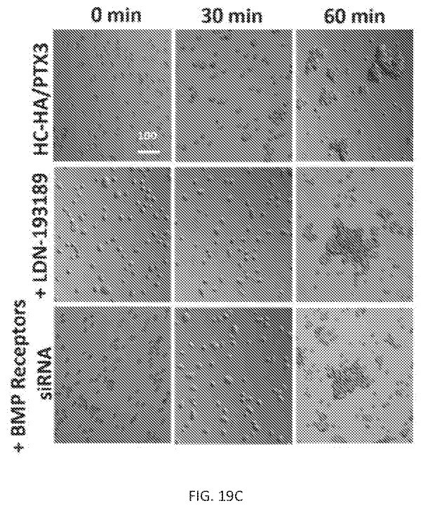

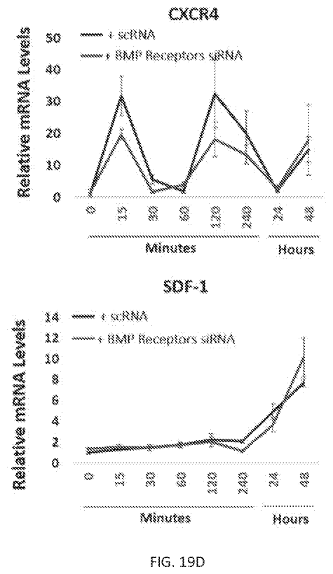

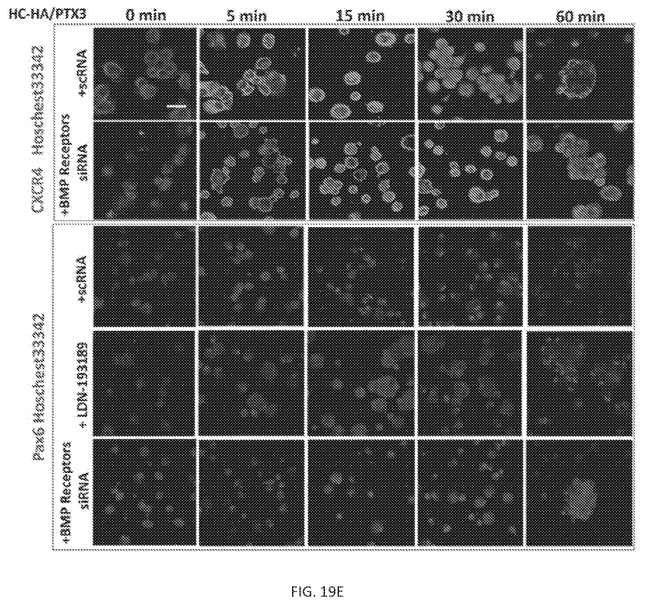

The BMP signaling promoted by soluble HC-HA/PTX3 was perturbed to determine whether BMP signaling was required for cell aggregation mediated by CXCR4/SDF-1 signaling. P10 LNC were pre-treated with or without LDN-193189, a small molecule BMP inhibitor or short interfering RNAs (siRNA) to BMP receptors, i.e., BMPR1A, BMPR1B, BMPR2, and Activin A receptor, type I (ACVR1) seeded on coated MG before adding soluble HC-HA/PTX3 in MESCM for another 48 h. Quantitative RT-PCR and immunofluorescence staining confirmed the efficiency of LDN-193189 (data not shown) and siRNAs to BMP receptors in reducing the transcript expressions of BMP receptors (FIG. 19A, ** p<0.01, n=3) and preventing nuclear staining of pSmad1/5/8 (FIG. 19B). However, phase contrast microscopy revealed that cell aggregation in P10 LNC by soluble HC-HA/PTX3 was not affect by either LDN-193189 or siRNAs to BMP receptors when compared to the control pre-treated with scrambled RNA (scRNA) (FIG. 19C). Quantitative RT-PCR further revealed that there was no significant difference in the expression level of CXCR4 and SDF-1 throughout 48 h when P10 LNC were pre-treated with either LDN-193189 or siRNAs to BMP receptors (FIG. 19D, P>0.1, n=3). Furthermore, immunofluorescence staining also showed that the transient nuclear translocation of CXCR4 and nuclear Pax6 staining were not affected (FIG. 19E). Collectively, these data indicated that cell aggregation, nuclear Pax6 staining, and activation of CXCR4/SDF-1 signaling by HC-HA/PTX3 were not affected when the canonical BMP signaling was inhibited.

### Discussion

Previously, early passaged P4 LNC have been shown to regain the expression of ESC markers lost during serial passage in coated MG when reseeded on immobilized HC-HA/PTX3. In this example, it was shown that passaged P10 LNC also regained the nuclear Pax6+ NC multipotent neural crest progenitor phenotype lost during serial passage when reseeded on immobilized HC-HA/PTX3 (FIGS. 15A-15D). Although both immobilized HC-HA/PTX3 and 3D MG promoted cell aggregation (FIGS. 15A-15D), such phenotypic reversal was unique to HC-HA/PTX3 because cell aggregation occurred as early as 60 min when soluble HC-HA/PTX3 was added in MESCM when P10 LNC were still cultured on coated MG, but not in their counterparts without HC-HA/PTX3 or reseeded on 3D Matrigel (FIGS. 16A-16C). The notion that cell aggregation induced by HC-HA/PTX3 was different from that by 3D MG was further supported by activation of CXCR4/SDF-1 signaling found in the former but not the latter. This was illustrated by notable upregulation of CXCR4 transcript at 15 min and nuclear translocation of CXCR4 at 15 and 30 min prior to cell aggregation facilitated by HC-HA/PTX3 (FIGS. 17A-17D). Suppression of CXCR4 by AMD3100 not only abolished upregulation of CXCR4 transcript and nuclear translocation of CXCR4 but also eliminated membranous and cytoplasmic staining of SDF-1 to interrupt CXCR4/SDF-1 signaling. Because it also abolished cell aggregation at 60 min, it was concluded that early cell aggregation facilitated by HC-HA/PTX3 was mediated by CXCR4/SDF-1 signaling. Such early cell aggregation promoted by HC-HA/PTX3 was pivotal to the phenotypic reversal to nuclear Pax6+ NC progenitor status as illustrated by the finding that addition of AMD3100 also prevented nuclear Pax6 staining and transcript upregulation of genes for NC markers (FIGS. 17A-17D). Because phenotypic reversal occurred only by HC-HA/PTX3 but not Matrigel®, of which both caused cell aggregation, it was speculated that cell aggregation triggered by homotypic CXCR4/SDF-1 signaling is unique. Future studies are needed to see if such a mechanism can be expanded to understand mesenchymal cell aggregation/condensation that is linked to promote organogenesis in tooth, bone, hair, skin and muscle or act as the key morphological event during the initiation reprogramming of skin fibroblast to induced pluripotent stem cells (iPSC).

CXCR4 is highly expressed in LNC subjacent to limbal basal epithelial stem/progenitors. but its expression also declined with serial passage on coated Matrigel (data not shown). Herein, it was noted nuclear translocation of CXCR4 soon after addition of HC-HA/PTX3. Furthermore, addition of AMD3100 prevented such transient nuclear translocation of CXCR4 and abolished cell aggregation and ensuing phenotypic reversal. Therefore, it was tempting to speculate that HC-HA/PTX3 activated CXCR4/SDF-1 signaling by nuclear translocation of CXCR4. As yet nuclear location of CXCR4 has been regarded as a strong indicator for high malignancy in several cancer cells and associated with HIF1α as a feed-forward loop to promote tumor growth and cancer metastasis in RCC cells. Because nuclear translocation of CXCR4 in LNC occurred much faster, i.e., 15 and 30 min after addition of HC-HA/PTX3, than what has been noted by sustained SDF-1 stimulation in cancer cells, future studies are needed to determine whether nuclear translocation of CXCR4 in LNC is promoted by HC-HA/PTX3 through a similar mechanism.

It has been shown that immobilized HC-HA/PTX3, but not 3D MG, activates BMP signaling in P4 LNC, which is required to maintain limbal epithelial SC quiescence. Herein, it was learned that BMP signaling evidenced by nuclear translocation of pSmad1/5/8 and upregulation of BMP ligands and receptors was also lost during serial passage (FIG. 18A) similar to nuclear Pax6 staining (FIGS. 14A-14B). Moreover, it was noted that both immobilized (not shown) and soluble HC-HA/PTX3 also activated BMP signaling in P10 LNC as evidenced by nuclear staining of pSmad1/5/8 at 30 min and upregulation of BMP4 and BMP6 transcript in a cyclic wave pattern before cell aggregation (FIGS. 18A-1B). Because BMP signaling is involved during the early stage of somatic cell reprogramming, which is also highlighted by cell aggregation and mesenchymal epithelial transition from single of adult mouse fibroblast cells and because CXCR4/SDF-1 signaling is linked to activating BMP signaling in mouse mesenchymal stem cells (MSC) to promote fracture wound healing, its role in the said phenotypic reversal to facilitate multipotency should be resolved. These data revealed that disruption of CXCR4/SDF-1 signaling by AMD3100 abolished the aforementioned BMP signaling promoted by HC-HA/PTX3 (FIGS. 18C-18D). In contrast, disruption of BMP signaling by LDN-193189 or siRNAs to BMP receptors neither affected cell aggregation mediated by CXCR4/SDF-1 signaling based on CXCR4 transcript expression and nuclear CXCR4 staining nor abolished nuclear Pax6 staining (FIGS. 19A-19E). Collectively, these results suggested that HC-HA/PTX3 promotes early cell aggregation by activating CXCR4/SDF-1 signaling, which is also required to activate BMP signaling in P10 LNC and that CXCR4/SDF-1 signaling is, but BMP signaling is not, pivotal in the phenotypic reversal of P10 LNC.

Previously, it has been reported that HC-HA/PTX3 purified from human AM exerts a broad anti-inflammatory and anti-scarring actions and supports LNC to ensure limbal epithelial SC quiescence. These actions collectively disclose the molecular mechanism explaining why cryopreserved amniotic membrane may promote regenerative healing. Herein, for the first time, evidence has been provided suggesting that HC-HA/PTX3 may also facilitate the reversal of aged LNC to regain their Pax6+ NC progenitor status to help explain why transplantation of AM augments the success of in vivo and ex vivo expansion of limbal SCs to treat corneal blindness caused by limbal SC deficiency. Because Pax6+ NC progenitors have wide differentiation potential into neurovascular cells, HC-HA/PTX3 might further support SC in many other neurovascular niches of the body.

While preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to elements of the embodiments of the disclosure described herein may be employed in practicing the disclosure. It is intended that the following claims define the scope of the disclosure and that methods and structures within the scope of these claims and their equivalents be covered thereby.

What is claimed is:

1. A method of reprogramming a cell having a first phenotype, comprising: contacting the cell with HC-HA/PTX3 for a time sufficient to reprogram the first phenotype of the cell to second phenotype.

2. The method of claim 1, wherein the second phenotype corresponds to a phenotype of an earlier cell in a cellular differentiation pathway.

3. The method of claim 1, wherein the cell is reprogrammed into an earlier cell in a cellular differentiation pathway.

4. The method of claim 1, wherein the cell is a cell differentiated from a progenitor cell.

5. The method of claim 4, wherein the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell.

6. The method of claim 4, wherein the progenitor cell is a neural crest progenitor.

7. The method of any one of claims 4-6, wherein the cell differentiated from the progenitor cell is a mesenchymal cell.

8. The method of claim 4, wherein the cell differentiated from the progenitor cell is a fibroblast, myofibroblast, keratocyte, epithelial cell, or limbal niche cell.

9. The method of claim 8, wherein the fibroblast is a myofibroblast, a dermal fibroblast, a corneal fibroblast, or a cardiac fibroblast.

10. The method of any one of claims 4-9, wherein the earlier cell is the progenitor cell.

11. The method of any one of claims 1-10, wherein the cell is present in a tissue following damage or degeneration of the tissue.

12. The method of claim 11, wherein the tissue is ocular, cardiac, skin, joint, spine, soft tissue, cartilage, bone, tendon, ligament, nerve, intervertebral disc, spinal cord, brain, or muscle tissue.

13. The method of claim 11, wherein the tissue is cardiac tissue.

14. The method of claim 11, wherein the tissue is ocular tissue.

15. The method of any one of claims 11-14, wherein the damage is the result of a burn, a laceration, ischemic tissue, a wound, an injury, an ulcer, radiation, chemotherapy, or a surgical incision.

16. The method of claim 15, wherein the injury is a myocardial infarction.

17. The method of any one of claims 1-16, wherein the HC-HA/PTX3 is comprised in a preparation of a fetal support tissue.

18. The method of claim 17, wherein the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof.

19. The method of claim 17, wherein the fetal support tissue is selected from placenta, placental amniotic membrane, umbilical cord, umbilical cord amniotic membrane, chorion, amnion-chorion, amniotic stroma, amniotic jelly, or a combination thereof.

20. The method of claim 17 or claim 19, wherein the fetal support tissue is frozen or previously frozen.

21. The method of any one of claims 17-20, wherein the fetal support tissue is substantially free of red blood cells.

22. The method of any one of claims 17-21, wherein the fetal support tissue comprises umbilical cord substantially free of a vein or artery.

23. The method of any one of claims 17-22, wherein the fetal support tissue comprises cells, substantially all of which are dead.

24. The method of any one of claims 17-23, wherein the fetal support tissue comprises umbilical cord amniotic membrane and at least a portion of Wharton's Jelly.

25. The method of any one of claims 17-24, wherein the fetal support tissue is cryopreserved, lyophilized, sterilized, or a combination thereof.

26. The method of any of claims 1-25, wherein the composition is a gel, a solution, or a suspension.

27. The method of any of claims 1-26, wherein the HC-HA/PTX3 is native HC-HA/PTX3, reconstituted HC-HA/PTX3, or a combination thereof.

28. The method of any one of claims 1-27, further comprising contacting the fibroblastic cell with TGFβ1.

29. A method of treating a condition characterized by unwanted fibroblastic cell differentiation in a subject in need thereof comprising, contacting a fibroblastic cell within a tissue affected by the condition in the subject with HC-HA/PTX3 for a period of time sufficient to reprogram a phenotype of the fibroblastic cell to a different phenotype, thereby treating the condition.

30. The method of claim 29, wherein the different phenotype corresponds to a phenotype of an earlier cell in a cellular differentiation pathway.

31. The method of claim 29, wherein the fibroblastic cell is reprogrammed into an earlier cell in a cellular differentiation pathway.

32. The method of claim 29, wherein the fibroblastic cell is a cell differentiated from a progenitor cell.

33. The method of claim 32, wherein the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell.

34. The method of claim 32, wherein the progenitor cell is a neural crest progenitor.

35. The method of any one of claims 32-34, wherein the cell differentiated from the progenitor cell is a mesenchymal cell.

36. The method of claim 32, wherein the cell differentiated from the progenitor cell is a fibroblast, myofibroblast, keratocyte, epithelial cell, or limbal niche cell.

37. The method of claim 36, wherein the fibroblast is a myofibroblast, a dermal fibroblast, a corneal fibroblast, or a cardiac fibroblast.

38. The method of claim 30 or claim 31, wherein the earlier cell is the progenitor cell.

39. The method of any one of claims claim 29-38, wherein the tissue is ocular, cardiac, skin, joint, spine, soft tissue, cartilage, bone, tendon, ligament, nerve, intervertebral disc, spinal cord, brain, or muscle tissue.

40. The method of any one of claims claim 29-38, wherein the tissue is ocular tissue.

41. The method of any one of claims claim 29-38, wherein the tissue is cardiac tissue.

42. The method of claim 41, wherein the condition is myocardial infarction.

43. The method of claim 42, wherein the contacting occurs during a stent placement surgical procedure.

44. The method of any one of claims 29-41, wherein the condition occurs as the result of a burn, a laceration, ischemic tissue, a wound, an injury, an ulcer, radiation, chemotherapy, or a surgical incision.

45. The method of any one of claims 29-44, wherein HC-HA/PTX3 is comprised in a preparation of fetal support tissue.

46. The method of claim 44, wherein the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof.

47. The method of claim 45, wherein the fetal support tissue is selected from placenta, placental amniotic membrane, umbilical cord, umbilical cord amniotic membrane, chorion, amnion-chorion, amniotic stroma, amniotic jelly, or a combination thereof.

48. The method of claim 45 or claim 47, wherein the fetal support tissue is frozen or previously frozen.

49. The method of any one of claims 45-48, wherein the fetal support tissue is substantially free of red blood cells.

50. The method of any one of claims 45-49, wherein the fetal support tissue comprises umbilical cord substantially free of a vein or artery.

51. The method of any one of claims 45-50, wherein the fetal support tissue comprises cells, substantially all of which are dead.

52. The method of any one of claims 45-51, wherein the fetal support tissue comprises umbilical cord amniotic membrane and at least a portion of Wharton's Jelly.

53. The method of any one of claims 45-52, wherein the fetal support tissue is cryopreserved, lyophilized, sterilized, or a combination thereof.

54. The method of any of claims 29-53, wherein the composition is a gel, a solution, or a suspension.

55. The method of any of claims 29-54, wherein the HC-HA/PTX3 is native HC-HA/PTX3, reconstituted HC-HA/PTX3, or a combination thereof.

56. The method of any one of claims 29-55, further comprising contacting the fibroblastic cell with TGFβ1.

57. A method of reversing a disease state in a tissue comprising, contacting the tissue with HC-HA/PTX3 for a time sufficient to reprogram diseased or unwanted cells in the tissue to a cell having a different phenotype, thereby reversing the disease state of the tissue.

58. The method of claim 57, wherein the different phenotype corresponds to a phenotype of an earlier cell in a cellular differentiation pathway.

59. The method of claim 57, wherein different phenotype corresponds to a phenotype of a progenitor cell.

60. The method of claim 59, wherein the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell.

61. The method of claim 57, wherein the unwanted cell is a fibroblast, myofibroblast, keratocyte, epithelial cell, or limbal niche cell.

62. The method of claim 61, wherein the fibroblast is a myofibroblast, a dermal fibroblast, a corneal fibroblast, or a cardiac fibroblast.

63. The method of any one of claims 57-62, wherein the disease or unwanted cell is present in a tissue following scarring, damage, or degeneration of the tissue.

64. The method of claim 63, wherein the tissue is ocular, cardiac, skin, joint, spine, soft tissue, cartilage, bone, tendon, ligament, nerve, intervertebral disc, spinal cord, brain, or muscle tissue.

65. The method of claim 63, wherein the tissue is cardiac tissue.

66. The method of claim 63, wherein the tissue is ocular tissue.

67. The method of any one of claims 57-66, wherein the HC-HA/PTX3 is comprised in a preparation of a fetal support tissue.

68. The method of claim 67, wherein the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof.

69. The method of claim 67, wherein the fetal support tissue is selected from placenta, placental amniotic membrane, umbilical cord, umbilical cord amniotic membrane, chorion, amnion-chorion, amniotic stroma, amniotic jelly, or a combination thereof.

70. The method of any one of claims 67-69, wherein the fetal support tissue comprises cells, substantially all of which are dead.

71. The method of any one of claims 67-70, wherein the fetal support tissue comprises umbilical cord amniotic membrane and at least a portion of Wharton's Jelly.

72. The method of any one of claims 67-71, wherein the fetal support tissue is cryopreserved, lyophilized, sterilized, or a combination thereof.

73. The method of any of claims 57-72, wherein the HC-HA/PTX3 is native HC-HA/PTX3, reconstituted HC-HA/PTX3, or a combination thereof.

74. A method of producing a progenitor cell from a differentiated cell comprising, contacting the differentiated cell with HC-HA/PTX3 for a time sufficient to reprogram the differentiated cell to a progenitor cell phenotype.

75. The method of claim 74, wherein the progenitor cell phenotype corresponds to a phenotype of an earlier cell in a cellular differentiation pathway.

76. The method of claim 74, wherein the progenitor cell phenotype corresponds the that of a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell.

77. The method of claim 74, wherein the differentiated cell is a fibroblast, myofibroblast, keratocyte, epithelial cell, or limbal niche cell.

78. The method of claim 77, wherein the fibroblast is a myofibroblast, a dermal fibroblast, a corneal fibroblast, or a cardiac fibroblast.

79. The method of any one of claims 74-77, wherein the differentiated cell is present in a tissue following scarring, damage, or degeneration of the tissue.

80. The method of claim 79, wherein the tissue is ocular, cardiac, skin, joint, spine, soft tissue, cartilage, bone, tendon, ligament, nerve, intervertebral disc, spinal cord, brain, or muscle tissue.

81. The method of claim 79, wherein the tissue is cardiac tissue.

82. The method of claim 79, wherein the tissue is ocular tissue.

83. The method of any one of claims 74-82, wherein the HC-HA/PTX3 is comprised in a preparation of a fetal support tissue.

84. The method of claim 83, wherein the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof.

85. The method of claim 83, wherein the fetal support tissue is selected from placenta, placental amniotic membrane, umbilical cord, umbilical cord amniotic membrane, chorion, amnion-chorion, amniotic stroma, amniotic jelly, or a combination thereof.

86. The method of any one of claims 83-85, wherein the fetal support tissue comprises cells, substantially all of which are dead.

87. The method of any one of claims 83-86, wherein the fetal support tissue comprises umbilical cord amniotic membrane and at least a portion of Wharton's Jelly.

88. The method of any one of claims 83-87, wherein the fetal support tissue is cryopreserved, lyophilized, sterilized, or a combination thereof.

89. The method of any of claims 74-88, wherein the HC-HA/PTX3 is native HC-HA/PTX3, reconstituted HC-HA/PTX3, or a combination thereof.

90. A method of regenerating a tissue comprising, reprogramming a first differentiated phenotype of a cell within a tissue to a progenitor phenotype, and differentiating the progenitor phenotype into a second differentiated phenotype, thereby regenerating the tissue.

91. The method of claim 90, wherein the progenitor cell phenotype corresponds to a phenotype of an earlier cell in a cellular differentiation pathway.

92. The method of claim 90, wherein the progenitor cell phenotype corresponds the that of a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell.

93. The method of claim 90, wherein the first differentiated cell is a fibroblast, myofibroblast, keratocyte, epithelial cell, or limbal niche cell.

94. The method of claim 93, wherein the fibroblast is a myofibroblast, a dermal fibroblast, a corneal fibroblast, or a cardiac fibroblast.

95. The method of any one of claims 93-94, wherein the first differentiated cell is present in the tissue following scarring, damage, or degeneration of the tissue.

96. The method of any one of claims 90-95, wherein the tissue is ocular, cardiac, skin, joint, spine, soft tissue, cartilage, bone, tendon, ligament, nerve, intervertebral disc, spinal cord, brain, or muscle tissue.

97. The method of any one of claims 90-95, wherein the tissue is cardiac tissue.

98. The method of any one of claims 90-95, wherein the tissue is ocular tissue.

99. The method of any one of claims 90-98, wherein the HC-HA/PTX3 is comprised in a preparation of a fetal support tissue.

100. The method of claim 99, wherein the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof.

101. The method of claim 99 or claim 100, wherein the fetal support tissue is selected from placenta, placental amniotic membrane, umbilical cord, umbilical cord amniotic membrane, chorion, amnion-chorion, amniotic stroma, amniotic jelly, or a combination thereof.

102. The method of any one of claims 99-101, wherein the fetal support tissue comprises cells, substantially all of which are dead.

103. The method of any one of claims 99-102, wherein the fetal support tissue comprises umbilical cord amniotic membrane and at least a portion of Wharton's Jelly.

104. The method of any one of claims 99-103, wherein the fetal support tissue is cryopreserved, lyophilized, sterilized, or a combination thereof.

105. The method of any of claims 90-104, wherein the HC-HA/PTX3 is native HC-HA/PTX3, reconstituted HC-HA/PTX3, or a combination thereof.

106. A composition comprising a) HC-HA/PTX3 and b) a therapeutic cell.

107. The composition of claim 106, wherein the therapeutic cell is a progenitor cell, a stem cells, or an induced pluripotent stem cell.

108. The composition of claim 107, wherein the progenitor cell is a neural crest progenitor, a hematopoietic progenitor cell, a mammary progenitor cell, an intestinal progenitor cell, a mesenchymal progenitor cell, an endothelial progenitor cell, a neural progenitor cell, an olfactory progenitor cell, a testicular progenitor cell, or a cardiovascular progenitor cell.

109. The composition of any one of claims 106-108, wherein HC-HA/PTX3 is comprised in a preparation of fetal support tissue.

110. The composition of claim 109, wherein the preparation is an extract of fetal support tissue, a fetal support tissue homogenate, a fetal support tissue powder, morselized fetal support tissue, pulverized fetal support tissue, ground fetal support tissue, a fetal support tissue graft, purified HC-HA/PTX3, reconstituted HC-HA/PTX3 or a combination thereof.

111. The composition of claim 109 or claim 110, wherein the fetal support tissue is selected from placenta, placental amniotic membrane, umbilical cord, umbilical cord amniotic membrane, chorion, amnion-chorion, amniotic stroma, amniotic jelly, or a combination thereof.

112. The composition of any one of claims 106-111, wherein the HC-HA/PTX3 is native HC-HA/PTX3, reconstituted HC-HA/PTX3, or a combination thereof.
